(19) United States
(12) Patent Application Publication
(10) Pub. No.: US 2023/0257805 A1
(43) Pub. Date: Aug. 17, 2023

(54) METHODS FOR LIGATION-COUPLED-PCR

(71) Applicant: Integrated DNA Technologies, Inc., Coralville, IA (US)

(72) Inventors: Vladimir Makarov, Ann Arbor, MI (US); Jordan Rosefigura, Ann Arbor, MI (US); Bita Lahann, Ann Arbor, MI (US); Robert D. Stedtfeld, Dexter, MI (US)

(73) Assignee: Integrated DNA Technologies, Inc., Coralville, IA (US)

(21) Appl. No.: 18/054,982

(22) Filed: Nov. 14, 2022

Related U.S. Application Data
(63) Continuation of application No. PCT/US21/32824, filed on May 17, 2021.
(60) Provisional application No. 63/025,738, filed on May 15, 2020.

(51) Int. Cl. C12Q 1/686

(52) U.S. Cl. CPC C12Q 1/686

(57) ABSTRACT

The present disclosure provides methods and kits for ligation-coupled PCR. Methods for performing ligation and PCR and, optionally, enzymatic digestion in a single closed tube are provided. Methods and kits for splint-mediated primer assembly and ligation-coupled PCR are also provided.

### CROSS-REFERENCE TO RELATED APPLICATIONS

This application is a continuation of International Patent Application No. PCT/US2021/032824, filed May 17, 2021, entitled Methods for Ligation-Coupled-PCR, the entire contents of which are hereby incorporated herein by reference. International Patent Application No. PCT/US2021/032824 claims the benefit of U.S. Provisional Application No. 63/025,738, filed May 15, 2020, which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 14, 2022, is named 85563-374183_Sequence_Listing.xml and is 138,952 bytes in size.

### BACKGROUND

An important prerequisite for analysis by next generation sequencing (NGS) is preparation of an NGS library. During this process, the nucleic acid of interest can first be fragmented, cDNA can be generated if starting with RNA, and for many targeted sequencing workflows, multiplexed PCR specific to loci of interest can be performed. Next, the substrate ends are repaired and ligated to NGS adapters to complete library synthesis. NGS adapters enable library amplification, clonal amplification of library molecules on a flow cell, and annealing of sequencing primers. NGS adapters also incorporate sample-specific indexes to allow multiplexed sequencing of libraries while the optional use of tags containing random nucleotides (6 or more N bases) enables unique molecular barcoding of each library molecule. NGS libraries can either be constructed using full length, indexed adapters during the ligation step, or alternatively, for Illumina sequencing, truncated adapters comprising the proximal adapter sequences nearest to the substrate can be ligated and then the sample-specific index and additional terminal adapter sequences are incorporated during library amplification using 5′ tailed indexing PCR primers that anneal to the truncated adapter sequences.

A traditional method for Illumina library preparation from fragmented DNA, cDNA or multiplexed PCR amplicons involves three steps that include: 1) DNA polishing, end repair and A-tailing to the 3′ ends of the substrate, 2) ligation of Y-shaped or stem-loop adapters to both substrate DNA strands, where the adapters comprise a double stranded region with a T nucleotide overhang at the 3′ end and two different single stranded adapter sequences, and 3) PCR amplification with a primer pair complementary to the two adapter sequences. This method is currently utilized in many commercially available kits such as TruSeq Nano DNA library kits (Illumina), NEBNext Ultra II DNA Library Prep Kit (New England Biolabs), and KAPA HyperPrep Kits (Roche). The use of DNA substrate A-tailing and T-overhang adapter ligation helps to reduce undesirable products such as chimeric library molecules containing 2 inserts and adapter dimers, while the use of asymmetric Y-shaped or stem-loop adapters enables attachment of the adapter to both strands of the A-tailed substrate in a single-step reaction without loss of library complexity.

Certain other methods use blunt ended adapters and avoid adapter-dimer formation by using adapters that ligate to only one DNA strand of the substrate but have no way of preventing chimeric library molecules and therefore limit DNA inputs to 50 ng. For example, in the methods used in SMARTer ThruPLEX DNA-Seq (Takara) and Ion Torrent DNA Library kits such as those used for AmpliSeq targeted multiplex PCR library construction (ThermoFisher), two blunt end adapters ligate to the 5′ ends of substrate DNA molecules, while the 3′ ends of the DNA substrate remain non-ligated and then become extended by a DNA polymerase in a subsequent step, prior to PCR amplification. The kits use a mixture of two different double stranded adapters, so only 50% of fully ligated DNA fragments comprise two different adapters which is required for sequencing, while 50% of fully ligated DNA fragments are lost by ligating two of the same adapter which cannot be sequenced, thus producing DNA, cDNA or targeted amplicon libraries with a 2-fold reduced yield and library complexity.

Other targeted sequencing library preparation methods such as CleanPlex (Paragon Genomics) utilize 5′ tailed target-specific primers, where each forward target-specific primer has a 5′ tail comprising a first truncated adapter sequence and each reverse target-specific primer has a 5′ tail comprising a second truncated adapter, such that the first target-specific multiplex PCR step is followed by a second indexing PCR step, where indexing primer pairs comprise a sample specific index and the terminal adapter sequences on a 5′ tail and are used to complete the first and second NGS adapter sequences. Although this is a simple workflow, it can produce a high proportion of primer dimers during the multiplex PCR step that would increase the cost of sequencing if the additional step of background cleaning was not incorporated to remove primer dimers, and the method also generates directional libraries that only sequence each library strand from one direction. Due to systematic sequencing errors, it is desirable to perform paired end sequencing of each strand from both directions for the most accurate sequence data quality for variant calling. Similar to this method, the AmpliSeq method described above also requires incorporation of an additional step to remove primer dimers prior to completion of the NGS library by end polishing, adapter ligation and optional PCR amplification steps, which introduces a number of post-multiplex PCR enzymatic steps.

Ideal methods for targeted sequencing produce non-directional, high complexity libraries and prevent primer dimer content in the final library from a minimal number of enzymatic steps. In the previously disclosed targeted multiplexed PCR library preparation commercialized as Accel-Amplicon and Swift Amplicon panels (as described in U.S. Pat. No. 10,316,359, incorporated by reference herein in its entirety), this workflow overcomes the library complexity limitation of double stranded adapter ligation, produces non-directional libraries and additionally prevents primer dimer accumulation without incorporating additional steps to the workflow. This method also enables contiguous coverage using primer pairs that produce overlapping amplicons in a single tube multiplexed PCR reaction, unlike AmpliSeq, CleanPlex and other multiplexed PCR targeted sequencing workflows where primer pairs that produce overlapping amplicons must be separated into two tubes. The Swift Amplicon method not only reduces sample tracking errors when processing large numbers of samples by utilizing a single tube workflow, but more importantly is suited to samples such as liquid biopsy/cell-free DNA from blood and other fluids, where input material is limited and sensitivity to detect low frequency variants is required. The single tube assay allows a higher copy number of input DNA molecules from limiting samples for greater sensitivity over assays requiring separate tubes.

Following the Swift multiplexed PCR, a purification step can be performed, and then an adapter ligation step can be performed on the purified multiplexed PCR amplicons, where the fully ligated molecules are 100% functional and non-directional, similar to Y-shaped adapters, although the method uses separate 5′ and 3′ adapters that independently ligate to the 5′ and 3′ ends of each strand of the target specific amplicons. Although this is both an efficient and easy workflow, because there is not an additional PCR amplification step, the libraries can only be accurately quantified by qPCR as fluorometric methods such as Qubit cannot distinguish partially ligated from fully ligated, functional molecules. Also, like Y-shaped adapters, the 5′ and 3′ single stranded adapter tails lead to migration artifacts on electrophoretic chips such as Bioanalyzer (Agilent), making quantification inaccurate using these methods as well. Therefore, a modification to the Swift Amplicon method to enable higher library yields is desired. Also, for high throughput applications, a modification to the method that does not result in libraries that require qPCR for accurate quantification prior to sequencing is also desirable. However, these modifications preferably do not introduce additional enzymatic or purification steps to the workflow to maintain a two-incubation method that is a preferred standard in the art for targeted library preparation using multiplexed PCR. The novel methods disclosed herein offer solutions to these proposed workflow improvements.

An ideal method for DNA NGS library preparation involves a minimal number of enzymatic steps (3 or less), a minimal number of purification steps (2 or less, including purification after PCR), close to 100% utilization of DNA, a broad range of DNA input (desirably, with a low threshold in the picogram—femtogram range and a high threshold in the microgram input range), lack of adapter-adapter dimers and DNA chimeras, and no requirement for adapter concentration adjustment present in many currently available kits.

In the previously disclosed NGS library preparation method commercialized as Accel-NGS 2S DNA Library kit as described in U.S. Pat. No. 10,316,357, incorporated by reference herein in its entirety, the workflow overcomes the library complexity limitation of double stranded adapter ligation library inherent to SMARTer, ThruPLEX, and Ion Torrent DNA Library kits and, additionally, prevents adapter-dimer and DNA chimera formation in a broad range of DNA concentration while using the same adapter concentration for both low and high input DNA samples, thus eliminating adapter concentration adjustment requirement present in TruSeq Nano, NEBNext Ultra II and KAPA Hyper Prep kits. It also demonstrates substantially lower AT/GC bias compared to other kits. Therefore, Accel-NGS 2S is a method of choice for low and precious samples such as DNA from ChIPSeq experiments and cell-free DNA but it has a larger number of enzymatic and purification steps.

Modification to the Accel-NGS 2S method is desirable to substantially reduce the number of enzymatic reactions and purification steps to be competitive with other methods and be attractive for larger number of applications. The novel methods disclosed herein offer solutions to these proposed workflow improvements.

### SUMMARY

The present disclosure provides methods and kits for ligation-coupled polymerase chain reaction (PCR) and methods and kits for splint-mediated primer generation.

Novel methods designed to increase library yields and overcome the requirement for qPCR quantification when using the Swift Amplicon multiplexed PCR targeting sequencing method referenced above and other methods are disclosed herein. The methods use indexing primers in a ligation-coupled PCR reaction. There are two methods that each utilize one of two different indexing primer designs that are commonly used in the art: indexing primers that comprise all sequences unique to each adapter but which omit the sequence common to both adapters at their 3′ terminus, and indexing primers that comprise the complete, full-length sequence of each adapter, including the sequence common to both adapters at their 3′ terminus. The shorter design reduces oligonucleotide synthesis costs while the longer design has a higher PCR efficiency as it can disrupt the secondary structure formed by the common adapter sequence and its reverse complement when priming denatured library molecules.

For either method, the Swift multiplexed PCR step can be performed as previously disclosed without any modification and is summarized below for reference, utilizing a plurality of target-specific primer pairs each comprising a 5′ tail sequence comprising a universal adapter sequence, and a universal primer complementary to the universal adapter sequence, where the universal primer contains a modification making it susceptible to cleavage by an endonuclease which is required for the subsequent adapter ligation step. The PCR utilizes a high fidelity proofreading polymerase that is tolerant of the modified base, where the first PCR cycles have elongated annealing times to allow the high complexity of target-specific primer pairs, each of which is at a low concentration, to create universal adapter tagged amplicons from their target sequences. Following the multiplex cycles (2 or more), PCR is continued with shorter annealing times for a second phase of amplification using the universal primer that anneals to the universal adapter flanking each target-specific amplicon. The universal primer is used at a relatively high concentration compared to the target-specific primers, so the universal primer supports the amplification reaction without additional primer dimer formation. Since the primer dimers that accumulate during the limited multiplexed cycles are significantly shorter in length than the desired amplicons, and due to their complementary universal adapter sequence, primer dimers are subject to stable secondary structure at the primer annealing temperature which results in less efficient amplification by the single universal primer. Similarly, when target-specific primer pairs that produce overlapping amplicons are used, the undesired short amplicon that results from the reverse primer of a first target specific amplicon and the forward primer of a second target specific amplicon is also less efficiently amplified by the single universal primer similar to primer dimers. This is due to the same stable secondary structure from their complementary universal adapter sequences at the primer annealing temperature, thus enabling a single tube assay with uniform target coverage. Without this special feature, the short amplicons would take over the PCR reaction as they can prime from the original template, each intended first and second amplicon as well as the short amplicon, leading to loss of coverage uniformity and a significantly higher cost of sequencing due to the imbalance of the amplicon products. For this reason, competing technologies separate primer pairs that produce overlapping amplicons into two multiplex PCR tubes to avoid this failure in achieving uniform target coverage.

Following the Swift multiplexed PCR, a bead-based purification step can be performed to remove unused primers and exchange buffer for the plurality of multiplexed PCR amplicons, each comprising the cleavable universal primer at each 5′ terminus. As shown in FIG. 1A, instead of following with the previously disclosed indexed adapter ligation, in one embodiment the purified multiplexed PCR products are combined with an endonuclease, a ligase, a PCR mastermix, including a DNA polymerase and deoxynucleotide triphosphates (dNTPs), and a full-length library indexing primer pair that includes the 3′ terminal sequence common to both adapters. By using full-length indexing primers, the primer corresponding to the 5′ adapter can be used as both an adapter for ligation and a primer for PCR amplification in the same ligation-coupled PCR reaction. In addition, an optional blocker oligonucleotide that is complementary to the 3′ end of the indexing primer that corresponds to the 3′ adapter can be pre-annealed to this primer. The blocker oligonucleotide prevents this primer from participating in the initial 5′ adapter ligation step at the first incubation temperature but has a Tₘ below the PCR annealing temperature or is inactivated so it does not block priming activity during PCR (see FIGS. 2,4). In this method, the reaction mixture is incubated under conditions sufficient to permit endonuclease cleavage of the incorporated universal primer on the 5′ ends of the amplicon substrates which then permits the 3′ end of the full length indexing primer comprising the 5′ adapter sequence to anneal to the 5′ end of the reverse complement of the universal adapter and ligate to the 5′ ends of the amplicon substrates, where both the endonuclease and ligation reactions occur in the PCR reaction mixture at a first temperature suitable for activity of both enzymes. Then the reaction mixture is heated to a high temperature to inactivate the endonuclease and ligase, denature the DNA substrate for PCR, and if using a hot start polymerase, activate the polymerase. PCR thermocycling is then performed for the required number of cycles to achieve the desired library yield using the indexing primers that anneal to the reverse complement of the universal adapter which comprises a truncated 3′ adapter sequence and the reverse complement of the full length 5′ adapter.

Following the ligation-coupled PCR, a bead-based purification step can be performed and then library quantification, pooling and sequencing can be performed. This novel method increases library yield making it suitable for low copy number samples with low kilobase target regions. It also results in libraries enriched for fully ligated, functional molecules with double-stranded adapters that can easily be quantified by fluorometric methods such as Qubit, or electrophoretic chips such as Bioanalyzer, in addition to qPCR. By combining adapter ligation with PCR amplification in a single closed tube, an additional purification step is avoided that would typically be required between adapter ligation and PCR, as well as avoiding addition of PCR reagents following the ligation reaction if a purification step was not required. In this regard, the novel workflow preserves the original workflow of two enzymatic incubations and two purification steps.

In an alternative embodiment shown in FIG. 1B, the purified multiplexed PCR products are combined with an endonuclease, a truncated 5′ adapter, a ligase, a PCR mastermix and a library indexing primer pair, each lacking the 3′ terminal sequence common to both adapters. In this embodiment a separate 5′ adapter is required for ligation as the indexing primers lack the common sequence of the adapter which is required for sequencing primer annealing. The reaction mixture is incubated under conditions sufficient to permit endonuclease cleavage of the universal primer incorporated on the 5′ ends of the amplicons during multiplex PCR, which then permits annealing of the 3′ portion of the 5′ truncated adapter to the 5′ portion of the reverse complement of the universal adapter and ligation to the 5′ ends of the amplicon substrate, where both the endonuclease and ligation reactions occur in the PCR reaction mixture at a first temperature suitable for activity of both enzymes. Then the reaction mixture is heated to a high temperature to inactivate the endonuclease and ligase, denature the DNA substrate for PCR, and if using a hot start polymerase, activate the polymerase. PCR thermocycling is then performed for the required number of cycles to achieve the desired library yield using indexing primers that anneal to the reverse complement of the universal adapter which comprises a truncated 3′ adapter sequence and the reverse complement of the truncated 5′ adapter. Additionally, the truncated 5′ adapter comprises secondary structure to allow it to participate in the ligation but prevent its activity as a primer during PCR so that it cannot truncate the full length 5′ adapter sequence from completed library molecules (see FIGS. 3A, 3D-3L, 5A-5C).

Following the ligation-coupled PCR, a purification step can be performed and then library quantification, pooling and sequencing can be performed. This novel method both increases library yield and results in libraries enriched for fully ligated, functional molecules with double-stranded adapters that can easily be quantified by fluorometric methods such as Qubit, or electrophoretic chips such as Bioanalyzer, in addition to qPCR. By combining adapter ligation with PCR amplification in a single closed tube, an additional purification step is avoided that would typically be required between adapter ligation and PCR, as well as avoiding addition of PCR reagents following the ligation reaction if a purification step was not required. In this regard, the workflow preserves the original workflow of two enzymatic reactions and two purification steps.

Additionally, both of the novel ligation-coupled PCR methods disclosed above can be combined with a simple enzymatic library normalization method commercialized as Normalase (and described in US Patent 10,961,562, incorporated by reference herein in its entirety). Addition of Normalase simplifies the library pooling step for multiplexed sequencing by avoiding quantification of individual library concentration and varied sample pooling volumes based on individual library concentration. Instead this method produces equimolar library yields that enables equal volume pooling of each library for a simple, high throughput post-library processing step prior to sequencing. The only requirements to incorporate this method into the two ligation-coupled PCR methods described above is that 1) each indexing primer must additionally comprise a a 5′ tail sequence that includes 3 or more consecutive ribonucleotide bases and two or more deoxynucleotides 5′ to the 3 or more consecutive ribonucleotide bases which can, by way of example but not limitation, be rU, 2) the DNA polymerase used in the amplification step must have 3′ to 5′ exonuclease proofreading activity in order to generate a 5′ overhang during PCR amplification (See FIG. 1C and 1D). Additionally, another primer pair, also comprising the 3 or more consecutive ribonucleotide bases and two or more deoxynucleotides 5′ to the 3 or more consecutive ribonucleotide bases on a 5′ tail, each corresponding to the terminal 5′ P5 and 3′ P7 adapter sequences, are included to increase PCR efficiency (18-221, 18-222 of Table 1, not shown in FIGS. 1C, 1D). As a result, the processed library molecules yielded after incubating the PCR mixture each comprise a 5′ overhang comprising the two or more deoxynucleotides of each primer and at least one of the 3 or more consecutive ribonucleotide bases. To obtain the desired target molar quantity of each NGS library from a starting quantity, the starting quantity must be greater than the target quantity, so the number of PCR cycles applied must achieve a library yield that is greater than the target quantity. Following PCR, a bead-based purification step is performed.

Normalase can then be performed without any modification to the previously disclosed enzymatic method and is summarized below for reference. The purified PCR products are combined with a ligase and a probe that is complementary to the 5′ overhang to yield a first enzymatic reaction mixture, wherein the probe is added to each library at an amount equal to the desired target molar quantity, and the first reaction mixture is incubated under conditions sufficient to permit ligation of the probe to the 3′ end(s) by annealing to the 5′ overhang portion(s) of the amplified library molecules, wherein the portion of the amplified library molecules ligated to the probe is the target molar quantity of processed library molecules. Next, since each library has the same target quantity of processed library molecules, equal volume pooling of each library to be co-sequenced is performed. Since the probe comprises a modification to provide resistance to digestion by an enzyme with exonuclease activity, the library pool is combined with an exonuclease in a second enzymatic reaction mixture under conditions sufficient to allow digestion of the processed library molecules that are not ligated to the probe, thereby isolating the selected target quantity of processed library molecules. The second enzymatic reaction mixture is then heat inactivated and the pool is ready for flow cell loading without an additional purification step. Optionally a qPCR quantification of the pool can be performed to confirm the desired final molarity to achieve a specified cluster density on the sequencing flow cell of choice.

In a separate embodiment, a ligation-coupled PCR reaction can be performed, where long PCR primers used to introduce additional sequences during amplification of a target substrate can be assembled by splint-mediated ligation prior to substrate amplification (see FIGS. 6A-6D). For example, the NGS library indexing primers compatible with subsequent enzymatic library normalization described above can be assembled by splint ligation to reduce oligo synthesis cost. Instead of ordering full length primers each up to 90 or more bases in length that differ only by the short, sample specific index sequence, for each primer of the pair, universal 5′ and 3′ primer subunits are synthesized and then only short oligonucleotide subunits comprising each unique sample specific index are synthesized, where two universal splint oligonucleotides link the 5′, index and 3′ primer subunits together for ligation. The splints comprise sequences complementary to portions of two primer subunits to bridge the 3′ terminus of the 5′ subunit to the 5′ terminus of the index subunit and the 3′ terminus of the index subunit to the 5′ terminus of the 3′ subunit. In this embodiment, any NGS library comprising both truncated adapters introduced by any method including adapter ligation or incorporation by PCR using 5′ tailed primers is combined with 3 subunits and 2 splint oligonucleotides for each indexing primer, a ligase and a PCR mastermix. In this method, the reaction mixture is incubated under conditions sufficient to permit annealing and ligation of the primer subunits in the PCR reaction mixture at a first temperature, then the reaction mixture is heated to a high temperature to inactivate the ligase, denature the NGS library substrate for PCR, and if using a hot start polymerase, activate the polymerase. PCR thermocycling is then performed for the required number of cycles to incorporate the index and remainder of the adapter sequences and achieve the desired library yield using the indexing primer ligation products that anneal to the two adapter sequences of the truncated NGS library. The two splint oligonucleotides comprise a 3′ blocking group to prevent priming activity during PCR, and the indexing and 3′ primer subunits require a 5′ phosphate for splint ligation. Without limitation, this method can be applied for assembly of any primers from any number of subunits and splints for amplification of any desired DNA substrate in a single closed tube.

In yet another embodiment, a ligation-coupled PCR reaction can be performed, where any product assembled by oligonucleotide splint ligation is also the substrate for PCR amplification in a single closed tube, such as for synthesis of long DNA products greater than the length limitations of individual oligonucleotide synthesis. For this method, individual oligonucleotide subunits are synthesized in tandem for one strand of the desired product and then splint oligonucleotides comprising sequences complementary to the 3′ portion and 5′ portion at the junction of tandem subunits are synthesized to bridge the tandem single stranded design. In this method, the subunits and splint oligonucleotides are combined with a ligase, a PCR mastermix and a forward primer comprising a sequence identical to the 5′ portion of the most 5′ subunit and a reverse primer that is complementary to the 3′ end of the most 3′ subunit. The reaction mixture is incubated under conditions sufficient to permit annealing and ligation of the oligonucleotide subunits in the PCR reaction mixture at a first temperature. Then the reaction mixture is heated to a high temperature to inactivate the ligase, and if using a hot start polymerase, activate the polymerase. PCR thermocycling is then performed for the required number of cycles using the forward and reverse primer to achieve the desired yield of double stranded product. The splint oligonucleotides comprise a 3′ blocking group to prevent priming activity during PCR, and oligonucleotide subunits require a 5′ phosphate for splint ligation. The subunits can be added to the reaction at a concentration too low to support PCR so that only the forward and reverse primers amplify the product to prevent unused subunit oligonucleotides from truncating the fully assembled product by priming during PCR.

The novel NGS library methods can also reduce the number of steps and lower the DNA input in Swift Accel-NGS 2S referenced above are disclosed herein, but should not be interpreted as limited thereto. The methods use indexing primers in a ligation-coupled-PCR reaction and there are at least four methods that each utilize one of the two different indexing primer designs that are commonly used in the art: indexing primers that comprise all sequences unique to each adapter but which omit the sequence common to both adapters at their 3′ terminus, and indexing primers that comprise the complete, full-length adapter, and two different sequence of each adapter, including the sequence common to both adapters at their 3′ terminus, and one of the two different adapter ligation chemistries used in the art: ligation of a blunt end adapter to a blunt substrate that is used to improve adapter ligation efficiency and library yield, and ligation of an adapter with a single T base overhang to a substrate with a single A base overhang that is used to prevent DNA chimera and adapter-dimer formation. The shorter indexing primer design reduces oligonucleotide synthesis costs while the longer indexing primer design has a higher PCR efficiency as it can disrupt the secondary structure formed by the common 3′ adapter sequence and its reverse complement when priming denatured library molecules. The use of the T/A adapter ligation chemistry during the first 3′ adapter ligation step in the sequential, two-step adapter ligation workflow, where the second 5′ adapter ligation occurs during ligation-coupled PCR reaction, almost completely eliminates formation of adapter-dimers and reduces the DNA input threshold down to femtogram level while the use of blunt end adapter ligation chemistry allows a low AT/GC bias.

For one exemplary method, DNA fragmentation and end repair are performed utilizing standard acoustic DNA fragmentation method such as Covaris and optimized end polishing protocol with the proofreading T4 DNA polymerase, which is followed by heat inactivation at 65° C. After heat inactivation of T4 DNA polymerase the DNA is combined with the blunt end 3′ adapter formed by annealing oligonucleotide 1 comprising uracil bases and oligonucleotide 2 phosphorylated at the 5′ end, T4 DNA ligase and a ligation buffer, and the first ligation reaction is performed. To prohibit adapter-dimer formation the base at the 3′ end of the oligonucleotide 1 is modified to prevent its ligation to the 5′ end of DNA, as disclosed before. Examples of such modified bases include, but not limited to are the dideoxynucleotides with two missing hydroxyl groups at the 2′ and 3′ positions within the ribose such as ddT, and the 3′-derivatives with one missing hydroxyl group at the 3′ position such as 3′-dT. 3′ adapter ligated to the 3′ end of DNA forms a nick with non-ligated 5′ end of DNA. Second end of the 3′ adapter is completely protected from ligation by the adapter design: omitting a 5′ phosphate group and placing a blocking group or non-ligatable base modification at the protruding 3′ end.

For another exemplary method, DNA fragmentation and end repair are performed utilizing standard acoustic DNA fragmentation method such as Covaris and optimized end polishing protocol with the proofreading T4 DNA polymerase, which is followed by incubation with Taq DNA polymerase at 65° C. to add a single A-base 3′ overhang to both DNA ends and heat inactivate T4 DNA polymerase. A-tailed DNA is combined with the 3′ adapter formed by annealing oligonucleotide 1 comprising uracil bases and phosphorylated at the 5′ end oligonucleotide 2 and comprising a single T (or U) base overhang at the 3′ end, T4 DNA ligase and a ligation buffer and the first ligation reaction is performed. As a result, the 3′ adapter with a single base overhang become attached to both DNA strands. Second end of the 3′ adapter is completely protected from ligation by the 3′ adapter design: omitting a 5′ phosphate group and placing a blocking group or non-ligatable base modification at the protruding 3′ end.

Following ligation of the 3′ adapter with a blunt or single base overhang, a bead-based purification step can performed to remove unused adapters, enzymes and exchange buffer. As shown on FIG. 1E and FIG. 1F, instead of following with the previously disclosed 5′ adapter ligation, SPRI bead purification and indexing PCR, in one embodiment the purified DNA products are combined with an endonuclease, a ligase, a PCR mastermix and a full-length library indexing primer pair that includes the 3′ terminal sequence common to both 3′ adapters. By using full-length indexing primers, the primer corresponding to the 5′ adapter can be used as both an adapter for ligation and a primer for PCR amplification in the same ligation-coupled-PCR reaction. In addition, an optional blocker oligonucleotide that is complementary to the 3′ end of the indexing primer that corresponds to the 3′ adapter may be pre-annealed to this primer. The blocker oligonucleotide prevents this primer from participating in the initial 5′ adapter ligation step at the first incubation temperature but has a Tₘ below the PCR annealing temperature or is inactivated so it does not block priming activity during PCR (see FIGS. 2B-2G and 4A-4E). In this method, the reaction mixture is incubated under conditions sufficient to permit endonuclease cleavage of the 3′ adapter oligonucleotide 8 or 9 containing uracil bases which then permits the 3′ end of the full length indexing primer comprising the 5′ adapter sequence to anneal to the 5′ end of the reverse complement of the universal adapter (first common nucleotide sequence) and ligate to the 5′ ends of DNA fragments, where both the endonuclease and ligation reactions occur in the PCR reaction mixture at a first temperature suitable for activity of both enzymes. Then the reaction mixture is heated to a high temperature to inactivate the endonuclease and ligase, denature the DNA substrate for PCR, and if using a hot start polymerase, activate the polymerase. PCR thermocycling is then performed for the required number of cycles to achieve the desired library yield using the indexing primers that anneal to the reverse complement of the universal adapter which comprises a truncated 3′ adapter sequence and the reverse complement of the full length 5′ adapter.

Following the ligation-coupled PCR, a bead-based purification step can be performed and then library quantification, pooling and sequencing can be performed. This novel method reduces the number of steps while preserving major advantages of the Accel-NGS 2S DNA workflow such as high DNA conversion rate, low adapter-dimers, broad range of DNA input and no requirement for adapter concentration adjustment for samples with variable input. In addition, the method utilizing 3′ adapter with the U or T base overhang permits library preparation from femtogram DNA inputs, the feature not offered by any available kit on the market. By combining adapter ligation with PCR amplification in a single closed tube, an additional purification step is avoided that would typically be required between adapter ligation and PCR, as well as avoiding addition of PCR reagents following the ligation reaction if a purification step was not required. In this regard, the novel workflow is similar by the number of enzymatic incubation and purification steps to most popular DNA library kits.

In the other embodiments shown in FIG. 1G and FIG. 1H, the purified 3′ adapter ligation products are combined with an endonuclease, a truncated 5′ adapter, a ligase, a PCR mastermix and a library indexing primer pair, each lacking the 3′ terminal sequence common to both adapters. In these embodiments a separate 5′ adapter is required for ligation as the indexing primers lack the common sequence of the adapter which is required for sequencing primer annealing. The reaction mixture is incubated under conditions sufficient to permit endonuclease cleavage of the 3′ adapter oligonucleotide 8 or 9 containing uracil bases, which then permits annealing of the 3′ portion of the 5′ truncated adapter to the 5′ portion of the 3′ adapter oligonucleotide 7 attached to the 3′ end of DNA, and its ligation to the 5′ ends of the DNA substrate, where both the endonuclease and ligation reactions occur in the PCR reaction mixture at a first temperature suitable for activity of both enzymes. Then the reaction mixture is heated to a high temperature to inactivate the endonuclease and ligase, denature the DNA substrate for PCR, and if using a hot start polymerase, activate the polymerase. PCR thermocycling is then performed for the required number of cycles to achieve the desired library yield using indexing primers that anneal to the sequence 7 which comprises a truncated 3′ adapter sequence. Additionally, the truncated 5′ adapter comprises secondary structure to allow it to participate in the ligation but prevent its activity as a primer during PCR so that it cannot truncate the full length 5′ adapter sequence from completed library molecules (see FIGS. 3,5).

Following the ligation-coupled PCR, a purification step can be performed and then library quantification, pooling and sequencing can be performed. This novel method reduces the number of steps while preserving major advantages of the Accel-NGS 2S DNA workflow such as high DNA conversion rate, low adapter-dimers, broad range of DNA input and no requirement for adapter concentration adjustment for samples with variable input. In addition, the method utilizing 3′ adapter with the U or T base overhang permits library preparation from femtogram DNA inputs, the feature not offered by any available kit on the market. By combining adapter ligation with PCR amplification in a single closed tube, an additional purification step is avoided that would typically be required between adapter ligation and PCR, as well as avoiding addition of PCR reagents following the ligation reaction if a purification step was not required. In this regard, the novel workflow is similar by the number of enzymatic incubation and purification steps to most popular DNA library kits.

Ligation-coupled-PCR methods for preparation DNA NGS library disclosed above can be combined with a simple enzymatic library normalization method commercialized as Normalase (and described in U.S. Pat. No. 10,961,562, incorporated by reference herein in its entirety). Addition of Normalase simplifies the library pooling step for multiplexed sequencing by avoiding quantification of individual library concentration and varied sample pooling volumes based on individual library concentration. Instead, this method produces equimolar library yields that enables equal volume pooling of each library for a simple, high throughput post-library processing step prior to sequencing. The only requirements to incorporate this method into the two ligation-coupled-PCR methods described above is that 1) each indexing primer must additionally comprise 3 or more consecutive ribonucleotide bases and two or more deoxynucleotides 5′ of the 3 or more consecutive ribonucleotide, 2) the DNA polymerase used in the amplification step must have 3′ to 5′ exonuclease proofreading activity in order to generate a 5′ overhang during PCR amplification (similar to the amplicon libraries shown in FIG. 1C and 1D).

Normalase can then be performed without any modification to the previously disclosed enzymatic method and is summarized below for reference. The purified PCR products are combined with a ligase and a probe that is complementary to the 5′ overhang to yield a first enzymatic reaction mixture, wherein the probe is added to each library at an amount equal to the desired target molar quantity, and the first reaction mixture is incubated under conditions sufficient to permit ligation of the probe to the 3′ end(s) by annealing to the 5′ overhang portion(s) of the amplified library molecules, wherein the portion of the amplified library molecules ligated to the probe is the target molar quantity of processed library molecules. Next, since each library has the same target quantity of processed library molecules, equal volume pooling of each library to be co-sequenced is performed. Since the probe comprises a modification to provide resistance to digestion by an enzyme with exonuclease activity, the library pool is combined with an exonuclease in a second enzymatic reaction mixture under conditions sufficient to allow digestion of the processed library molecules that are not ligated to the probe, thereby isolating the selected target quantity of processed library molecules. The second enzymatic reaction mixture is then heat inactivated and the pool is ready for flow cell loading without an additional purification step. Optionally a qPCR quantification of the pool can be performed to confirm the desired final molarity to achieve a specified cluster density on the sequencing flow cell of choice.

### BRIEF DESCRIPTION OF THE DRAWINGS

FIG. 1A depicts an exemplary workflow where a partially double-stranded DNA substrate is produced by multiplex PCR using target-specific primers and universal primer with dU bases and endonuclease cleavage which yields 3′ overhangs, followed by 5′ adapter ligation and indexing PCR using two indexing primers which each have a 3′ terminal portion that is complementary to a 5′ portion of the 3′ overhangs (a first common nucleotide sequence), where one of the indexing primers can function as the 5′ adapter while the other cannot because it is annealed to a blocker oligonucleotide.

FIG. 1B depicts an exemplary workflow where a partially double-stranded DNA substrate is produced by multiplex PCR using target-specific primers and universal primer with dU bases and endonuclease cleavage which yields 3′ overhangs, followed by 5′ adapter ligation using a hairpin adapter which has a 3′ terminal sequence complementary to a 5′ portion of the 3′ overhangs (the first common nucleotide sequence), and indexing PCR using two indexing primers which do not include 3′ terminal portions complementary to a 5′ portion of the 3′ overhangs (a first common nucleotide sequence), but where the second indexing primer has a 3′ terminal portion complementary to a second common nucleotide sequence that is 3′ of the first common nucleotide sequence. The hairpin includes a portion of the first indexing primer which allows for subsequent PCR using the first indexing primers after the first PCR cycle.

FIG. 1C depicts an exemplary workflow where the indexing primers include a 5′ normalization tail that allows for library normalization after the ligation-coupled PCR. FIG. 1C discloses (T)₁₂(rU)₄ as SEQ ID NO: 1.

FIG. 1D depicts an exemplary workflow where the indexing primers include a 5′ normalization tail that allows for library normalization after the ligation-coupled PCR. FIG. 1D discloses (T)₁₂(rU)₄ as SEQ ID NO: 1.

FIG. 1E depicts a NGS library preparation method that involves DNA repair, ligation of the 3′-adapter with blunt end, and ligation-coupled PCR with full size indexing primers and primer blocker.

FIG. 1F depicts a NGS library preparation method that involves DNA repair, A-tailing, ligation of the 3′-adapter with U overhang, and ligation-coupled PCR with full size indexing primers and primer blocker.

FIG. 1G depicts a NGS library preparation method that involves DNA repair, ligation of the 3′-adapter with blunt end, and ligation-coupled PCR with truncated indexing primers and hairpin 5′ adapter.

FIG. 1H depicts a NGS library preparation method that involves DNA repair, A-tailing, ligation of the 3′-adapter with U overhang, and ligation-coupled PCR with truncated indexing primers and hairpin 5′ adapter.

FIG. 1I depicts a comparison of adapter-dimer formation in different NGS library protocols.

FIG. 2A depicts an exemplary workflow where a partially double-stranded DNA substrate is produced by endonuclease cleavage which yields 3′ overhangs, followed by 5′ adapter ligation and indexing PCR using two indexing primers which each have a 3′ terminal portion that is complementary to a 5′ portion of the 3′ overhangs (a first common nucleotide sequence), where either of the indexing primers can function as the 5′ adapter.

FIG. 2B depicts an exemplary workflow where a partially double-stranded DNA substrate is produced by endonuclease cleavage which yields 3′ overhangs, followed by 5′ adapter ligation and indexing PCR using two indexing primers which each have a 3′ terminal portion that is complementary to a 5′ portion of the 3′ overhangs (a first common nucleotide sequence), where one of the indexing primers can function as the 5′ adapter while the other cannot because it is annealed to a blocker oligonucleotide.

FIG. 2C depicts further details of (a) a linear blocker comprising a low Tₘ so its inactive during PCR or (b) a hairpin blocker that becomes inactivated during PCR when using Illumina TruSeq adapters. FIG. 2C discloses SEQ ID NOs: 78-80, 78-80, 78-79, 81-82, 78-79, and 83-84, respectively, in order of appearance.

FIG. 2D depicts Illumina TruSeq adapter sequences for aspects of the multiplex PCR, endonuclease cleavage of the incorporated universal primer, linear blocker 5 with 3 mismatches pre-annealed to the 3′ adapter indexing primer (i7), and ligation of 3′ end of the 5′ adapter indexing primer (i5) to the 5′ portion of the reverse complement of the universal adapter sequence on the substrate amplicons. FIG. 2D discloses SEQ ID NOs: 85, 85-87, 42, 78-79, 89, 86, 78-79, 89, 87, 42, and 86, respectively, in order of appearance.

FIG. 2E depicts Illumina TruSeq adapter sequences for aspects of the multiplex PCR, endonuclease cleavage of the incorporated universal primer, linear blocker 5 with a 6T insertion pre-annealed to the 3′ adapter indexing primer (i7), and ligation of 3′ end of the 5′ adapter indexing primer (i5) to the 5′ end of the substrate amplicons. FIG. 2E discloses SEQ ID NOs: 85, 85-87, 42, 78-79, 89, 86, 78-79, 89, 87, 42, and 86, respectively, in order of appearance.

FIG. 2F depicts a linear blocker that in addition to a Tm reducing T*G mismatch has 3 degradable U bases. FIG. 2F discloses SEQ ID NOs: 78-79, 49, 78-79, 90, 78-79, and 91, respectively, in order of appearance.

FIG. 2G depicts a NGS library preparation that involves DNA repair, ligation of a blunt end 3′-adapter and ligation-coupled PCR with full size indexing primers and i7 primer blocker containing one mismatch and several degradable dU bases. FIG. 2G discloses SEQ ID NOs: 92, 92-93, 93, 87, 42, 78-79, 90, 93, 78-79, 90, 87, 42, and 93, respectively, in order of appearance.

FIG. 3A depicts ligation-coupled PCR reaction with truncated indexing primers i5 and i7 lacking 13 bases at the 3′ terminus and a linear 5′ adapter.

FIG. 3B ligation-coupled PCR reaction with truncated indexing primers i5 and i7 lacking 13 bases at the 3′ terminus and a truncated hairpin 5′ adapter.

FIG. 3C further depicts that the stem loop truncated adapter 5 also comprises a non-replicable modification represented by the black circle within the loop sequence so formation of completely replicated hairpin products are prevented.

FIG. 3D depicts the undesired result when the stem loop truncated adapter lacks a non-replicable modification within the loop sequence.

FIG. 3E depicts a specific embodiment using a TruSeq Illumina adapter workflow when a hairpin truncated adapter comprising an internal non-replicable C3 spacer is used for ligation-coupled-PCR. FIG. 3E discloses SEQ ID NOs: 85, 85-86, 94, 42, 87, 95, 78, 96-97, 86-87, 95, 78, 96-97, 94, 42, 78, 96, and 93, respectively, in order of appearance.

FIG. 3F depicts an alternative embodiment using a TruSeq Illumina adapter workflow where hairpin truncated adapter with internal non-replicable C3 spacer is used for ligation-coupled-PCR. FIG. 3F discloses SEQ ID NOs: 98, 98, 86, 94, 53, 87, 95, 78, 96-97, 86-87, 95, 78, 96-97, 94, 42, 78, 96, and 93, respectively, in order of appearance.

FIG. 3G depicts a Nextera Illumina adapter workflow is used with a hairpin truncated adapter, full-length indexing primers and a linear blocker for ligation-coupled-PCR.

FIG. 3G discloses SEQ ID NOs: 99, 99-102, 87, 103, 78, 104-105, 100, 87, 103, 78, 104-105, 101, 103, and 100, respectively, in order of appearance.

FIG. 3H depicts another Nextera Illumina adapter workflow where a hairpin truncated adapter is used with indexing primers lacking the common adapter sequence for ligation-coupled-PCR. In this example, the universal amplicon primer containing dU bases replaces all 5 T deoxynucleotides, but the 3′ terminal 9 base sequence is retained due to absence of T bases when USER cleavage is performed. FIG. 3H discloses SEQ ID NOs: 99, 99-102, 87, 106, 78, 107-108, 100, 87, 106, 78, 107-108, 101, 103, 78, 107, and 109, respectively, in order of appearance.

FIG. 3I depicts a NGS library preparation that involves DNA repair, A-tailing, ligation of a 3′-adapter with U base overhang, and ligation-coupled PCR with truncated indexing primers and hairpin 5′ adapter with 13b 3′ overhang. FIG. 31 discloses SEQ ID NOs: 51, 51, 24, 93-94, 42, 87, 95, 78, 96, 93, 87, 95, 94, 42, 78, 96, and 93, respectively, in order of appearance.

FIG. 3J depicts a NGS library preparation that involves DNA repair, ligation of a blunt end 3′-adapter, and ligation-coupled PCR with truncated indexing primers and hairpin 5′ adapter with 13b 3′ overhang. FIG. 3J discloses SEQ ID NOs: 92, 92-93, 93-94, 42, 87, 95, 78, 96, 93, 87, 95, 94, 42, 78, 96, and 93, respectively, in order of appearance.

FIG. 3K depicts a NGS library preparation that involves DNA repair, A-tailing, ligation of a 3′-adapter with T base overhang, and ligation-coupled PCR with truncated indexing primers and hairpin 5′ adapter with l11b 3′ overhang. FIG. 3K discloses SEQ ID NOs: 52, 52, 24, 93-94, 53, 87, 95, 78, 96, 93, 87, 95, 94, 42, 78, 96, and 93, respectively, in order of appearance.

FIG. 3L depicts a NGS library preparation that involves DNA repair, A-tailing, ligation of a 3′-adapter with T base overhang and ligation-coupled PCR with full size indexing primers, blocker containing one mismatch and several degradable U bases and hairpin 5′ adapter with 10b overhang. FIG. 3L discloses SEQ ID NOs: 110, 110-111, 111, 101-102, 87, 103, 78, 104-105, 112, 87, 103, 78, 104-105, 101, 103, and 100, respectively, in order of appearance.

FIG. 4A depicts exemplary structures of the linear blocker and hairpin blocker.

FIG. 4B depicts workflows using the blocker oligonucleotide.

FIG. 4C depicts the blocker and one of the indexing primers.

FIG. 4D depicts a workflow using the hairpin blocker.

FIG. 4E depicts an exemplary workflow for the hairpin blocker showing that the extended stem can increase the melting temperature of the extended hairpin.

FIG. 5A depicts the hairpin adapter and a first indexing primer.

FIG. 5B depicts a workflow using the hairpin adapter.

FIG. 5C depicts a workflow using the hairpin adapter.

FIG. 6A depicts an exemplary workflow of splint-mediated primer assembly..

FIG. 6B depicts details of a method where Normalase compatible primers are assembled that comprise (T)₁₂(rU)₄ (SEQ ID NO: 1) at their 5′ terminus. FIG. 6B discloses (T)₁₂(rU)₄ as SEQ ID NO: 1.

FIG. 6C depicts assembly of a TruSeq Illumina indexing primer with the (T)₁₂(rU)₄ (SEQ ID NO: 1) sequence compatible with downstream enzymatic normalization. FIG. 6C discloses SEQ ID NOs: 113-118, and 116-117, respectively, in order of appearance.

FIG. 6D depicts a ligation-coupled-PCR workflow when primer assembly of i5 TruSeq Illumina indexing primer of 4C. above is combined with assembly of a corresponding i7 indexing primer comprising the 3′ adapter sequence, annealing of a hairpin blocker to the i7 primer, and ligation of the i5 primer to an amplicon substrate comprising a truncated 3′ adapter (following endonuclease cleavage, not shown). FIG. 6D discloses SEQ ID NOs: 113-117, 119-122, 83, 82, 93, 118, 116-118, 116-117, 93, 123, 122, 83, and 82, respectively, in order of appearance.

FIG. 7 depicts results of experimental Example 1. ITS1 rRNA amplicon coverage for Candida albicans observed in IGV where reads originating from the forward primer are plotted on the top and reads from the reverse primer are plotted on the bottom of the IGV plot.

FIG. 8 depicts results of experimental Example 3. Over 90% assembly of indexing primer by ligation was observed using T3 DNA ligase for splint ligation under conditions optimized for PCR, whereas T4 DNA ligase was inefficient.

FIG. 9 discloses SEQ ID NOs: 44, 49, 44, 50, 44, 124, 44, 125, 44, and 46, respectively, in order of appearance.

FIG. 9 depicts double stranded DNA structures formed by indexing primer i7 with different primer blockers described in Example 5.

FIG. 10A depicts structure of the 3′ and 5′ adapters for NGS libraries described in Example 6. FIG. 10A discloses SEQ ID NOs: 52, 51, 24, 24, 45, 53, 45, and 42, respectively, in order of appearance.

FIG. 10B depicts the Picard plots for NGS libraries described in Example 6 (NGS library prep A 50 ng).

FIG. 10C depicts the Picard plots for NGS libraries described in Example 6 (NGS library prep A 250 ng).

FIG. 10D depicts the Picard plots for NGS libraries described in Example 6 (NGS library prep B 50 ng).

FIG. 10E depicts the Picard plots for NGS libraries described in Example 6 (NGS library prep B 250 ng).

FIG. 11A depicts a Bio Analyzer trace for libraries prepared from pictogram amount of DNA by methods described in Example 6.

FIG. 11B depicts a Bio Analyzer trace for libraries prepared from pictogram amount of DNA by methods described in Example 6.

FIG. 11C depicts a Bio Analyzer trace for libraries prepared from femtogram amount of DNA by methods described in Example 6.

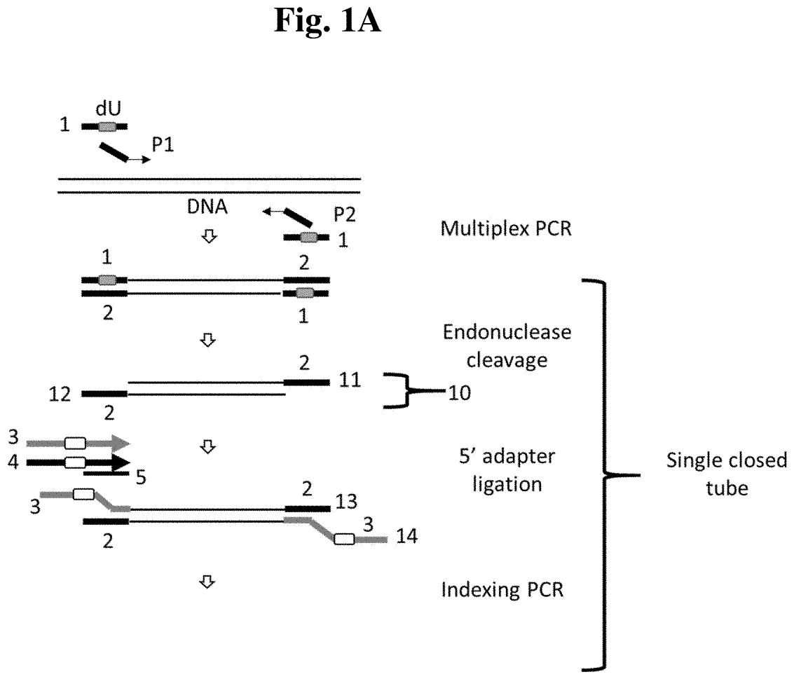

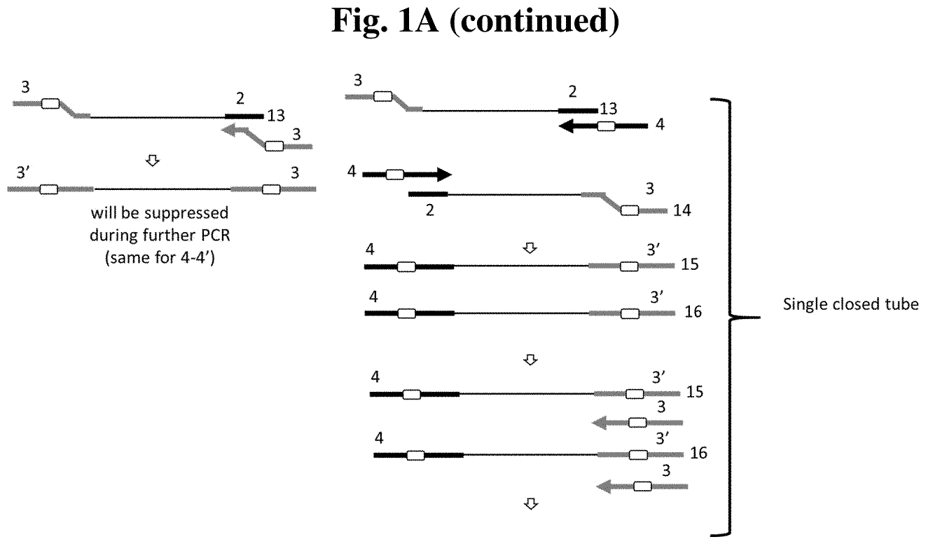

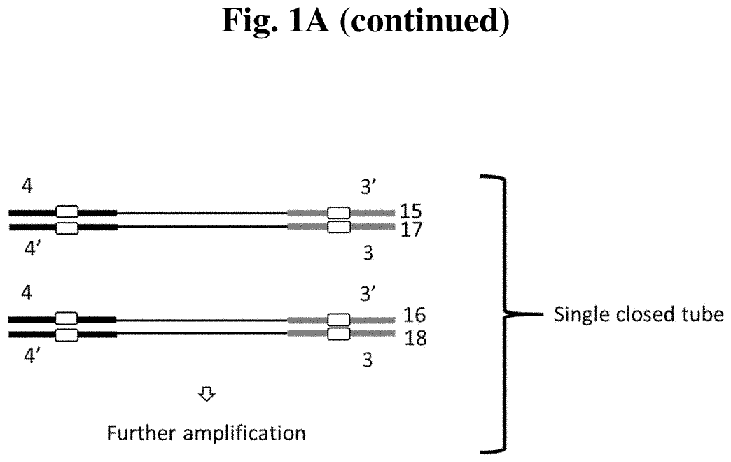

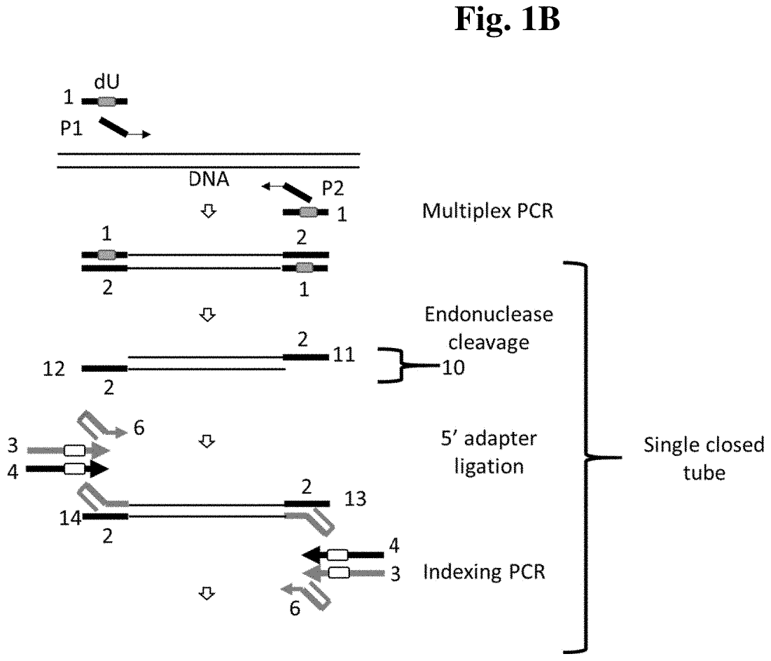

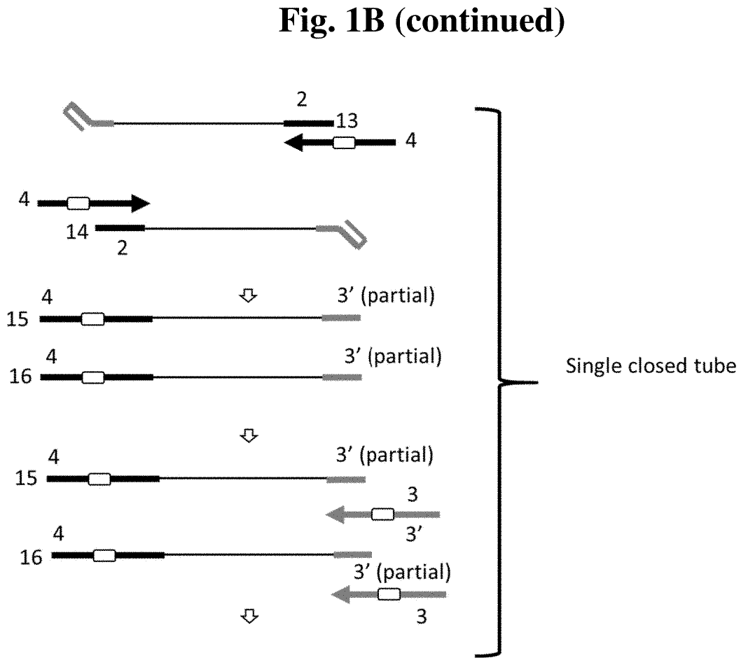

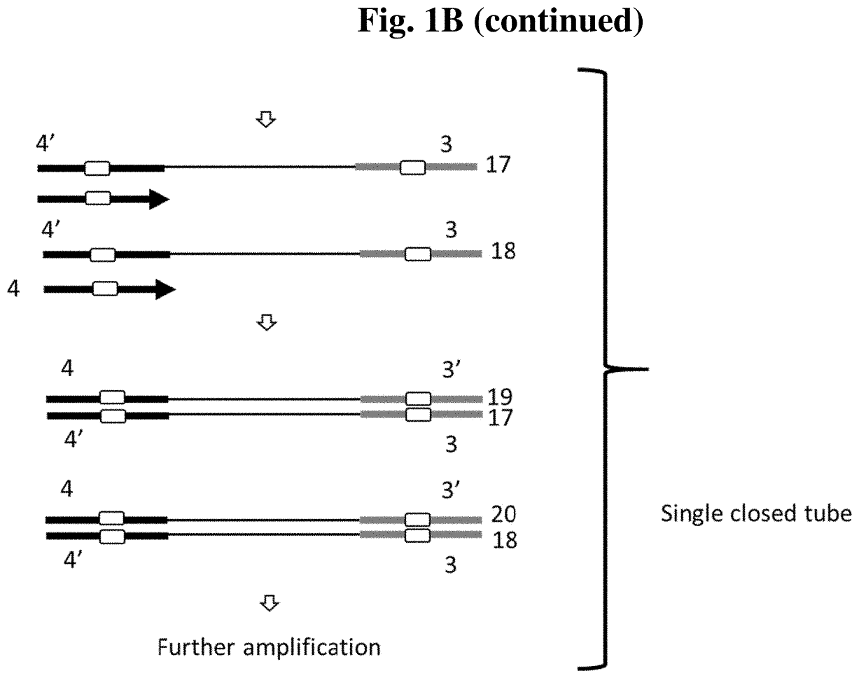

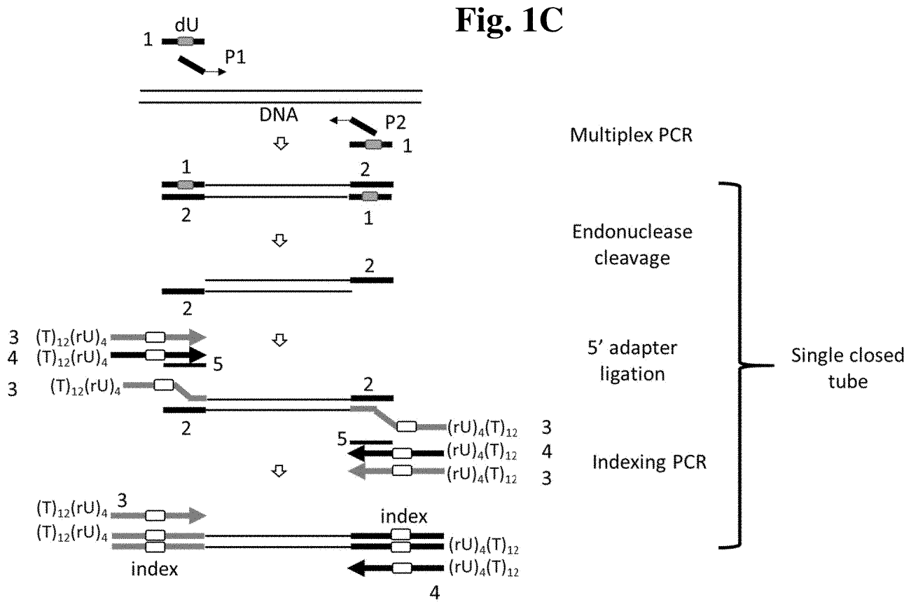

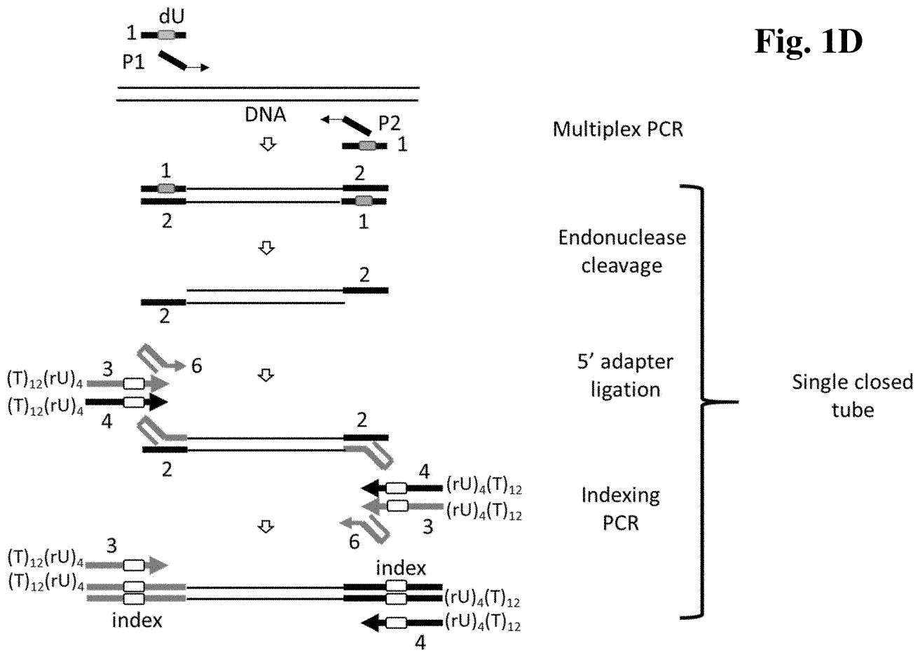

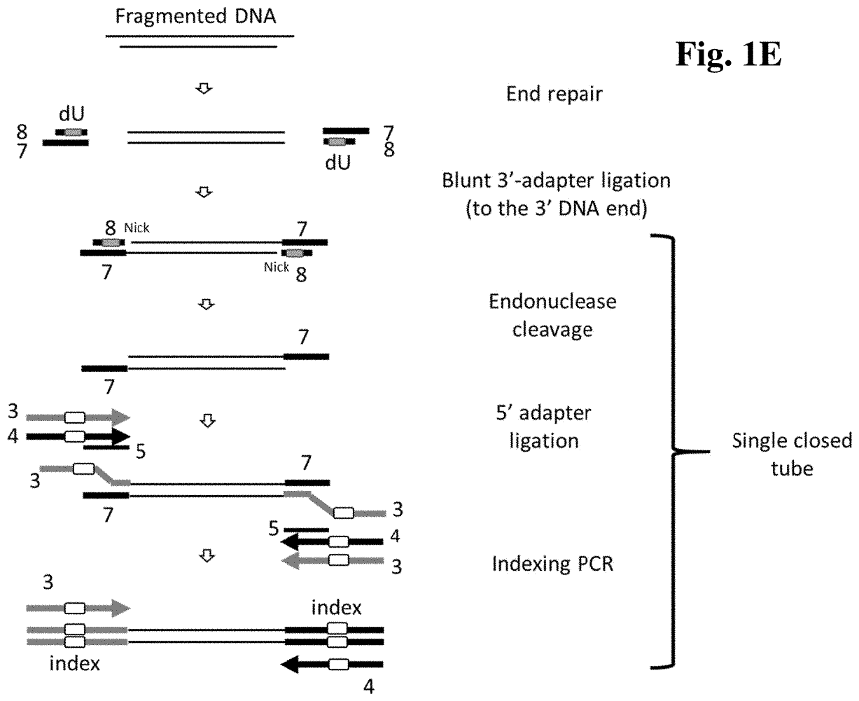

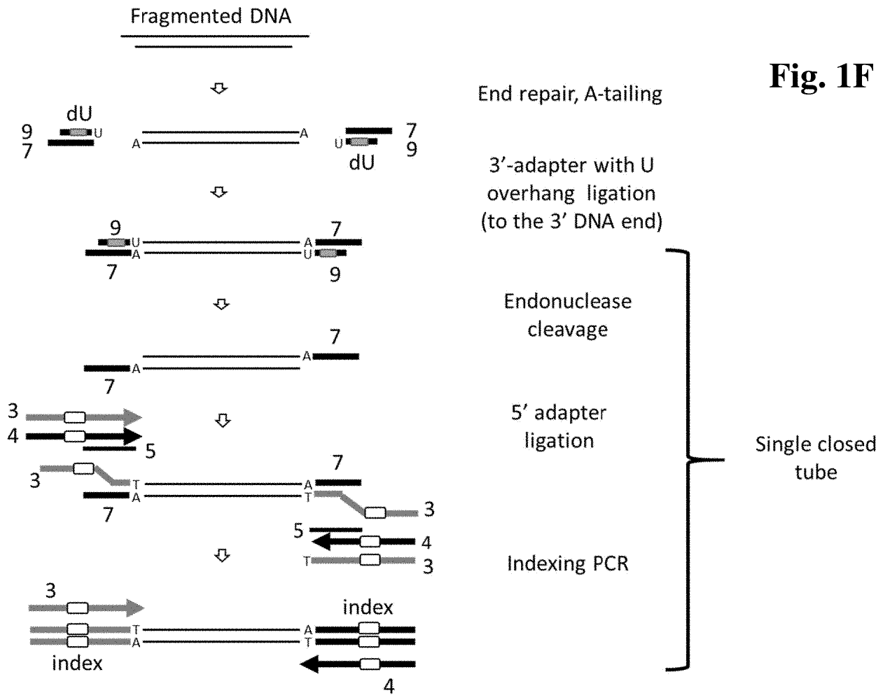

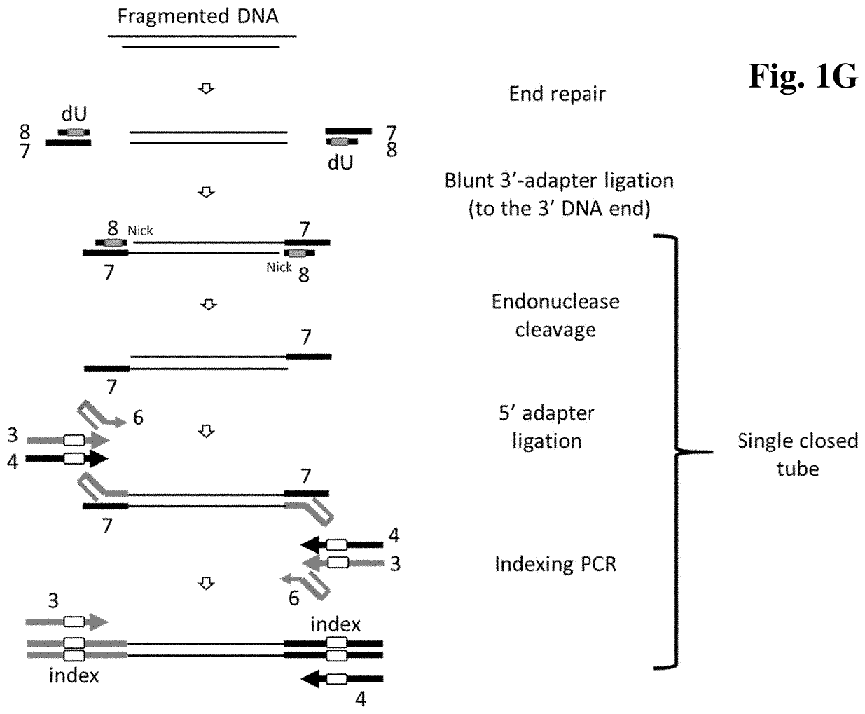

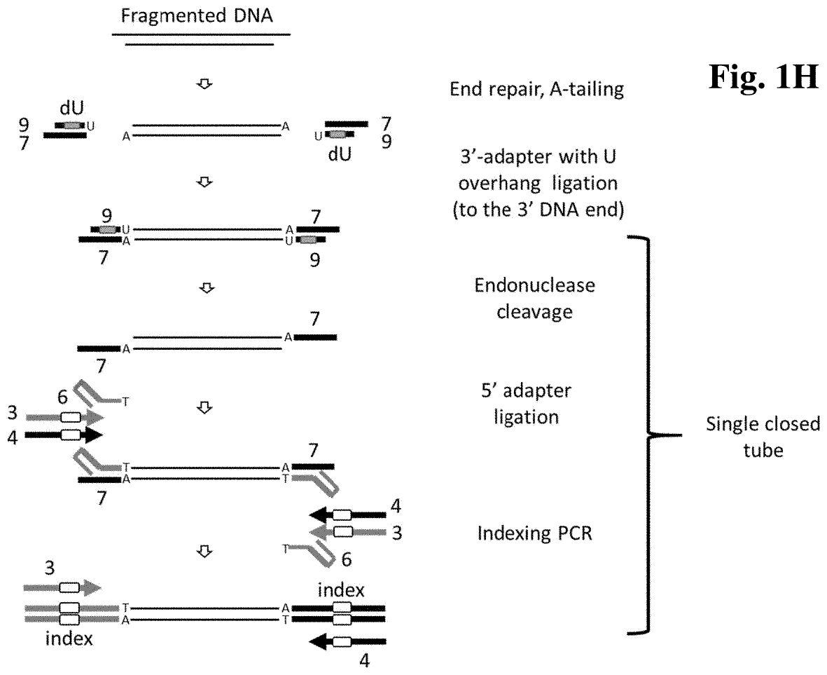

In the drawings, it should be understood that where a reaction is indicated to occur in a single closed tube and the bracket is on a continued page of the figure, all of the steps in all of the brackets are to be understood to be occurring in the same closed tube. By way of example, FIG. 1A spans three pages, however, all of the steps after the multiplex PCR are to be understood as being in the same “single closed tube” even though separate brackets appear on each page.

### DETAILED DESCRIPTION

The present disclosure describes particular embodiments and with reference to certain drawings, but the subject matter is not limited thereto. The drawings described are only schematic and are non-limiting. In the drawings, the size of some of the elements may be exaggerated or distorted and not drawn on scale for illustrative purposes. Where the elements of the disclosure are designated as “a” or “an” in first appearance and designated as “the” or “said” for second or subsequent appearances unless something else is specifically stated.

The present disclosure will provide description to the accompanying drawings, in which some, but not all embodiments of the subject matter of the disclosure are shown. Indeed, the subject matter may be embodied in many different forms and should not be construed as limited to the embodiments set forth herein, rather, these embodiments are provided so that this disclosure satisfies all the legal requirements.

Definitions

Certain terminology is used in the following description for convenience only and is not limiting. Certain words used herein designate directions in the drawings to which reference is made. Unless specifically set forth herein, the terms “a,” “an” and “the” are not limited to one element, but instead should be read as meaning “at least one.” As used herein “another” means at least a second or more. The terminology includes the words noted above, derivatives thereof and words of similar import.

The use of the term “or” in the claims is used to mean “and/or” unless explicitly indicated to refer to alternatives only or the alternatives are mutually exclusive.

Use of the term “about”, when used with a numerical value, is intended to include +/−10%. For example, if a number of amino acids is identified as about 200, this would include 180 to 220 (plus or minus 10%).

As used herein, a “low magnesium buffer” can be any buffer that has a magnesium level low enough to be suitable for PCR. By way of example, but not limitation, the low magnesium buffer can have 1-2 mM magnesium or less.

Unless otherwise defined, all technical and scientific terms used herein have the same meaning as commonly understood by one of ordinary skill in the art.

The present disclosure provides method for ligation-coupled PCR. Such methods can be used to add adapters to DNA substrates for next generation sequencing (NGS) and other applications. Methods are also provided for splint-mediated primer assembly and use by ligation-coupled PCR. Kits are also provided for performing the methods of the present disclosure.

It should be understood that the methods disclosed herein can be used with any starting DNA substrate that includes, or is capable of being treated to include, two 3′ overhangs or with any partially-double stranded DNA substrate having the two 3′ overhangs. This can then enable subsequent ligation of the 5′ adaptor and PCR amplification of the ligated, double-stranded DNA substrate by the primers. While the embodiments disclosed herein are, in some instances, disclosed with respect to certain upstream processing methods, they should not be construed as limited solely to those methods.

A method of ligation-coupled-PCR is disclosed where either i) a PCR substrate is assembled from DNA subunits by ligation and amplified by PCR, ii) a PCR primer is assembled from DNA subunits by ligation and used for amplification by PCR, or a combination thereof, where the ligation and amplification reactions occur in a single, closed tube. Regarding i), the PCR substrate is generated by ligation of tandem oligonucleotides linked by complementary splint oligonucleotides. Alternatively, the PCR substrate is a truncated NGS library comprising a first adapter with cleavable bases, where an endonuclease cleaves one strand of the first adapter to enable annealing and ligation of the second adapter. Regarding ii), the PCR primer is also assembled by ligation of tandem oligonucleotides linked by complementary splint oligonucleotides.

In one embodiment the multiplexed amplicon workflow for ligation-coupled PCR utilizing full-length indexing primers 3 (first indexing primer) and 4 (second indexing primer) including the common adapter sequence at their 3′ termini is shown (FIG. 1A). Universal-tailed target-specific primers P1 and P2 (representative of multiple primer pairs) are shown with universal primer 1, which is complementary to the universal tails of P1 and P2, comprising cleavable dU bases in the multiplexed PCR. The second reaction combines endonuclease cleavage of incorporated universal primer 1 by USER enzyme to yield a partially double-stranded substrate DNA molecule 10 which includes a first strand 11 and a second strand 12, ligation of the 5′ adapter indexing primer 3 (first indexing primer) to the 5′ end of substrate DNA 10 after annealing to at least a portion of the reverse complement of the universal primer sequence 2, a first common nucleotide sequence that is present in both 3′ overhangs at a 5′ end of the overhang, and optional pre-annealed linear blocker 5 to prevent the 3′ adapter indexing primer 4 (second indexing primer) from participating in the ligation reaction (since the universal primer 1 comprises the same sequence as the 3′ portion of the 3′ adapter 4 (second indexing primer), which is followed by indexing PCR to amplify the library and complete the adapter sequences, in a single closed tube. Once the 5′ adapter 3 (first indexing primer) is ligated, it can yield a third strand 13 and a fourth strand 14. Because the blocker prevents ligation of the second indexing primer 4, e.g. because it can have a higher melting temperature (Tₘ) than the temperature during the ligation reaction, there is no ligation of the second indexing primer 4 to the substrate DNA molecule 10. In the first PCR cycle, while either the first indexing primer 3 or second indexing primer 4 can anneal to at least a portion of the reverse complement of the universal primer sequence (which includes the first common nucleotide sequence) 2, if the first indexing primer 3 anneals and is extended, it will yield a product with the first indexing primer and its complement at opposite ends, which can be suppressed in further PCR cycles. Thus, where the second indexing primer 4 anneals to at least a portion of the reverse complement of the universal primer sequence 2, it will produce a fifth strand 15 from the third strand 13 as a template, and a sixth strand 16 from the fourth strand 14 as a template. Since both the fifth strand 15 and sixth strand 16 each have the reverse complement of the first indexing primer 3, the first indexing primer 3 can anneal to each in the next PCR cycle and extend to yield a seventh strand 17 and an eighth strand 18 which are complementary to the fifth strand 15 and the sixth strand 16, respectively. In further PCR cycles, these double-stranded products can be further amplified by the first indexing primer 3 and second indexing primer 4. FIG. 1A depicts all steps from endonuclease cleavage through indexing PCR occurring in a single closed tube, however, it should be understood that the steps from 5′ adapter ligation through indexing PCR may be performed in a single closed tube regardless of if endonuclease cleavage (or other enzymatic processing) is also performed. For all figures and embodiments, it should be understood that the cleavage steps to yield the partially double-stranded DNA substrate can occur in a single closed tube with the ligation and PCR or can be separately performed before the ligation-coupled PCR. In some embodiments, the enzymatic processing to obtain the partially double-stranded DNA substrate can be performed in a prior step or can be performed as a part of the workflow in a single closed tube as shown in FIG. 1A.

In another embodiment, the targeted amplicon workflow for ligation-coupled-PCR utilizing indexing primers 3 and 4 lacking the common adapter sequence at their 3′ termini is depicted (FIG. 1B). Universal-tailed target-specific primers P1 and P2 (representative of multiple primer pairs) are shown with universal primer 1 comprising cleavable dU bases in the multiplexed PCR. The second reaction combines endonuclease cleavage of incorporated universal primer 1 by USER enzyme, ligation of the truncated 5′ hairpin adapter 6 to the end of amplicon DNA after annealing to at least a portion of the reverse complement of the universal primer sequence 2 (e.g., to the first common nucleotide sequence), which is followed by indexing PCR using indexing primers 3 and 4 to amplify the library and complete the adapter sequences, in a single closed tube. Secondary structure of the hairpin 5′ adapter prevents its activity as a primer during PCR so that it does not truncate the 5′ adapter from completed library molecules.

The method of FIG. 1A can be modified for downstream enzymatic library normalization by utilizing indexing primers 3 and 4 that further comprise a 5′ tail sequence where the 5′ tail sequence can include 4 consecutive U ribonucleotide (rU) bases and can include 5′ adjacent to the 12 T deoxynucleotides (SEQ ID NO: 2) (FIG. 1C). An additional pair of normalization primers comprising the 5′ (T)₁₂(rU)₄ tail (SEQ ID NO: 1) and terminal P5 and P7 adapter sequences can also be included in the reaction to increase PCR efficiency (oligonucleotides 18-221, 18-222 from Table 1, primers not shown in FIG. 1C).

The method of FIG. 1B can also be modified for downstream enzymatic library normalization by utilizing indexing primers 3 and 4 that further comprise 5′ tail sequence where the 5′ tail sequence can include 4 consecutive U ribonucleotide (rU) bases and can include 5′ adjacent to the 12 T deoxynucleotides (SEQ ID NO: 2) (FIG. 1D). An additional pair of 5′ normalization primers comprising the 5′ (T)₁₂(rU)₄ tail (SEQ ID NO: 1) and terminal P5 and P7 adapter sequences can also be included in the reaction to increase PCR efficiency (oligonucleotides 18-221, 18-222 from Table 1, primers not shown in FIG. 1D).

In one embodiment the truncated NGS library workflow for ligation-coupled PCR is utilizing full-length indexing primers 3 and 4 (FIG. 1E). In this workflow fragmented DNA is subjected to the end repair with a polishing DNA polymerase such as T4 or T7 DNA polymerase and the ligation of a 3′ adapter with blunt end formed by oligonucleotides 7 and 8 with the blunt end, where oligonucleotide 8 has one or more cleavable bases such as U base and a modified nucleotide at the 3′ terminus of the blunt end that does not participate in the ligation reaction (for example, modifications where the ribose is missing from the 3′ hydroxyl or both 3′ and 2′ hydroxyl groups), and oligonucleotide 7 has a phosphate at the 5′ terminus of the blunt end that participates in the ligation reaction. After bead purification, the final reaction combines endonuclease cleavage of oligonucleotide 8 by USER enzyme, ligation of the 5′ adapter (indexing primer 3) to the 5′ end of DNA after annealing to the reverse complement of the 3′ adapter oligonucleotide 7, and optional pre-annealed linear blocker 5 to prevent the indexing primer 4 from participating in the ligation reaction (since the indexing primer 4 comprises the same sequence as the 3′ portion of the indexing primer 3), which is followed by indexing PCR to amplify the library and complete the adapter sequences, in a single closed tube.

The method of FIG. 1E can be modified to use with A-tailed DNA. The 3′ end A-tailing can be achieved by incubation with DNA polymerases lacking 3′ proofreading activity such as (3′exo-) Klenow fragment of DNA polymerase I or Taq DNA polymerase followed by ligation of the 3′ adapter with U base overhang at the 3′ end formed by oligonucleotides 7 and 8, where oligonucleotide 9 has at least two cleavable U bases including the U base at the 3′ terminus that participates in the ligation reaction, and oligonucleotide 7 has a phosphate at the 5′ terminus of the blunt end that also participates in the ligation reaction. After bead purification, the final reaction combines endonuclease cleavage of oligonucleotide 9 by USER enzyme, ligation of the 5′ adapter indexing primer 3 to the 5′ end of DNA after annealing to the reverse complement of the 3′ adapter oligonucleotide 7, and optional pre-annealed linear blocker 5 to prevent the indexing primer 4 from participating in the ligation reaction (since the indexing primer 4 comprises the same sequence as the 3′ portion of the indexing primer 3), which is followed by indexing PCR to amplify the library and complete the adapter sequences, in a single closed tube as shown on FIG. 1F.

In another embodiment the truncated NGS library workflow for ligation-coupled PCR is utilizing indexing primers 3 and 4 lacking the common adapter sequence at their 3′ termini (FIG. 1G). In this workflow fragmented DNA is subjected to the end repair with a polishing DNA polymerase such as T4 or T7 DNA polymerase and the ligation of a 3′ adapter with blunt end formed by oligonucleotides 7 and 8, where oligonucleotide 8 has one or more cleavable bases such as U base and a modified nucleotide at the 3′ terminus of the blunt end that does not participate in the ligation reaction (for example, modifications where the ribose is missing the 3′ hydroxyl or both 3′ and 2′ hydroxyl groups), and oligonucleotide 7 has a phosphate at the 5′ terminus of the blunt end that participates in the ligation reaction. After bead purification, the final reaction combines endonuclease cleavage of oligonucleotide 8 by USER enzyme, ligation of the truncated hairpin 5′ adapter 6 to the 5′ end of DNA after annealing to the reverse complement of the 3′ adapter oligonucleotide 7, which is followed by indexing PCR using indexing primers 3 and 4 to amplify the library and complete the adapter sequences, in a single closed tube. Secondary structure of the hairpin 5′ adapter prevents its activity as a primer during PCR so that it does not truncate the 5′ adapter from completed library molecules.

The method of FIG. 1G can be modified to use with A-tailed DNA. The 3′ end A-tailing can be achieved by incubation with DNA polymerases lacking 3′ proofreading activity such as (3′exo-) Klenow fragment of DNA polymerase I or Taq DNA polymerase followed by ligation of a 3′ adapter with U base overhang at the 3′ end formed by oligonucleotides 7 and 8, where oligonucleotide 8 has at least two cleavable U bases including the U base at the 3′ terminus that participate in the ligation reaction, and oligonucleotide 7 has a phosphate at the 5′ terminus of the blunt end that also participates in the ligation reaction. After bead purification, the final reaction combines endonuclease cleavage of oligonucleotide 8 by USER enzyme, ligation of the truncated 5′ hairpin adapter 6 to the 5′ end of DNA after annealing to the reverse complement of the 3′ adapter oligonucleotide 7, which is followed by indexing PCR to amplify the library and complete the adapter sequences, in a single closed tube as shown on FIG. 1H. Secondary structure of the hairpin 5′ adapter prevents its activity as a primer during PCR so that it does not truncate the 5′ adapter from completed library molecules.

NGS library preparation methods utilizing a 3′ adapter with the blunt end and the ligation-coupled PCR step (FIG. 1E and FIG. 1G) offer a substantial improvement over the Accel-NGS 2S DNA library workflow by reducing the number of enzymatic steps from 5 to 3 and purification steps from 5 to 2 while preserving the major advantages of Accel-NGS 2S DNA library preparation over other available kits such as high library yield, no requirement for adapter concentration adjustment when varying DNA input, and very low AT/GC bias. The only limitation of the described methods is a formation of chimeric DNA at DNA input concentrations above 50-100 ng.

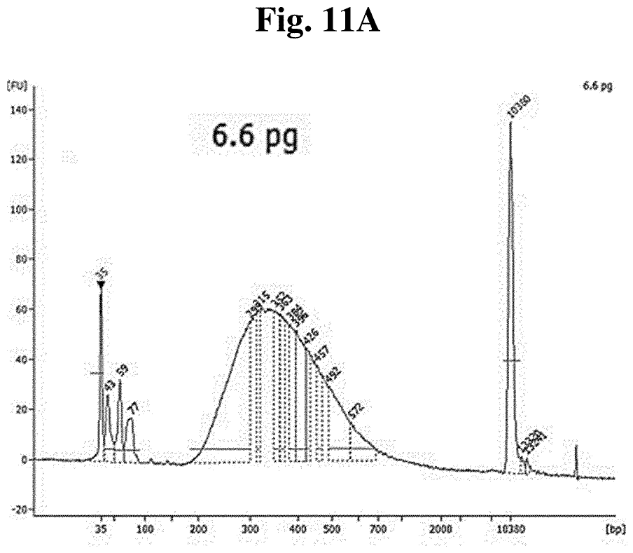

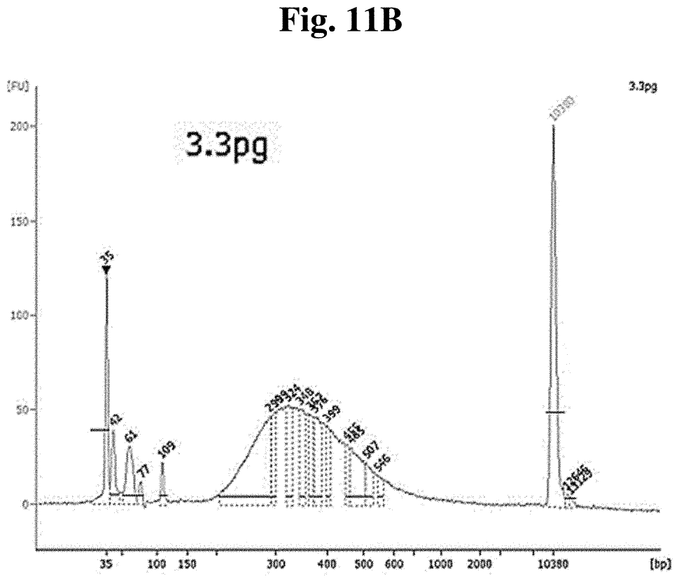

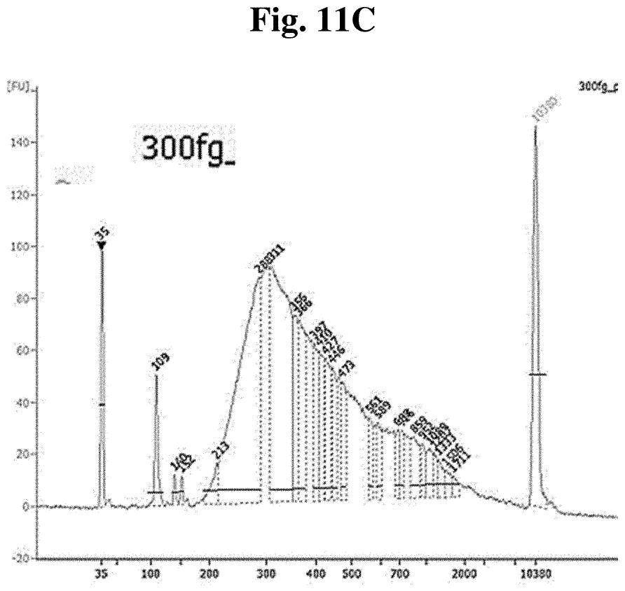

NGS library preparation methods utilizing a 3′ adapter with the U or T overhang and the ligation-coupled PCR step (FIG. 1F and FIG. 1H) offer a substantial improvement over the Accel-NGS 2S DNA library workflow by reducing the number of enzymatic steps from 5 to 3 and purification steps from 5 to 2 while preserving all advantages of Accel-NGS 2S DNA library preparation such as high library yield, lack of DNA chimera, very broad DNA input range and no requirement for adapter concentration adjustment when varying DNA input. The lack of adapter dimers and, as a result, the ability to work with extremely low, femtogram amount of DNA is a unique property of the workflows shown on FIGS. 1F and 1H that strongly differentiates them from all previously described NGS library methods. As it is shown in FIG. 11 (a), strong suppression of adapter-dimer formation in the protocol shown in FIG. 1G (as well as 1H) originates from the fact that any oligonucleotide 7 remaining after 3′ adapter ligation and purification after annealing to the primer oligonucleotide 3 would form an adapter with a T base overhang that is not capable of self-ligating when present at low concentrations. As it is shown in FIG. 1I (b, c), the adapter-dimer formation is still possible for the Accel-NGS 2S (b) and the NGS library protocols utilizing Y adapter (c): in the case of Accel-NGS 2S library, formation of the adapter-dimer happens during the second ligation reaction because self-ligation of the blunt adapter (formed by annealing of the carry-over 3′ adapter 2 and the adapter primer 3) can occur even at a relatively low adapter concentration; in the case (c) it occurs due to high T4 DNA ligase and Y adapter concentration, despite the presence of the T*T mismatch between the adapter ends.

In several embodiments, such as amplicons and NGS library with a T or U base overhang, the endonuclease used in the ligation-coupled PCR reaction is the USER enzyme from New England Biolabs or Uracil Cleavage System from Qiagen, or any other mix of uracil DNA glycosylase (UDG) and endonuclease VIII. In the NGS library embodiments that utilize blunt end 3′ adapter with the ddT, 3′-dT, ddG or 3′-dG base at the 3′ end, where is no covalent link between the 3′ end of the 3′ adapter oligonucleotide and the 5′ end of DNA, destabilization and release of the adapter oligonucleotide can be achieved by incubation with the UDG enzyme only. UDG enzyme does not produce breaks within the oligonucleotide 1 but creates a number of abasic sites that are sufficient for the reduction of the annealing temperature below 37° C. and dissociation of the oligonucleotide 8 from the complementary 3′ adapter oligonucleotide 7 to allow annealing and ligation of the 5′ adapter indexing primer 3 to the DNA end (FIG. 1G).

Methods Using a Blocker Oligonucleotide

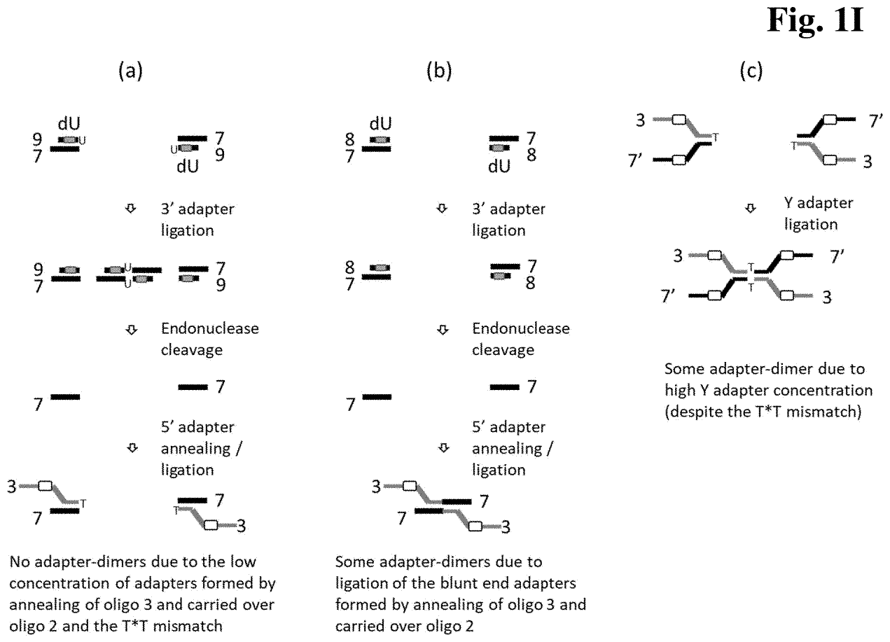

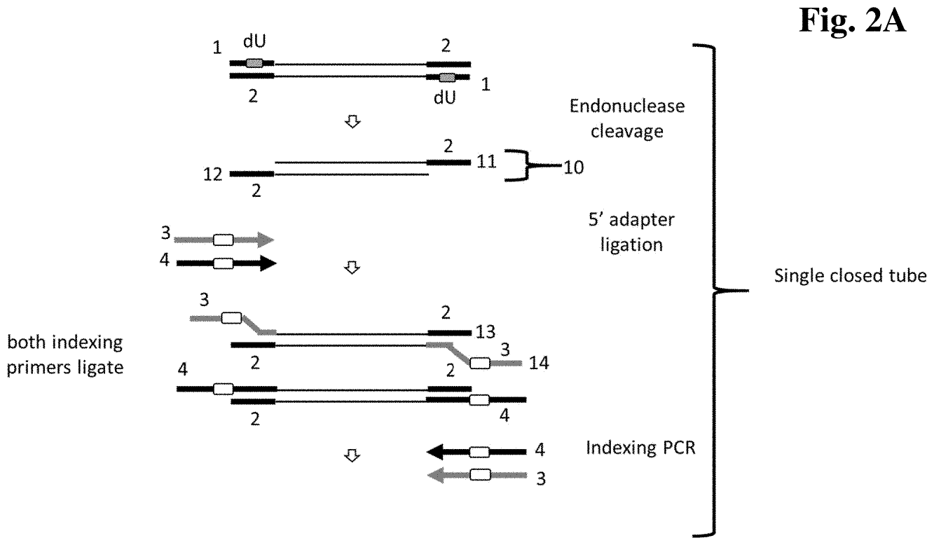

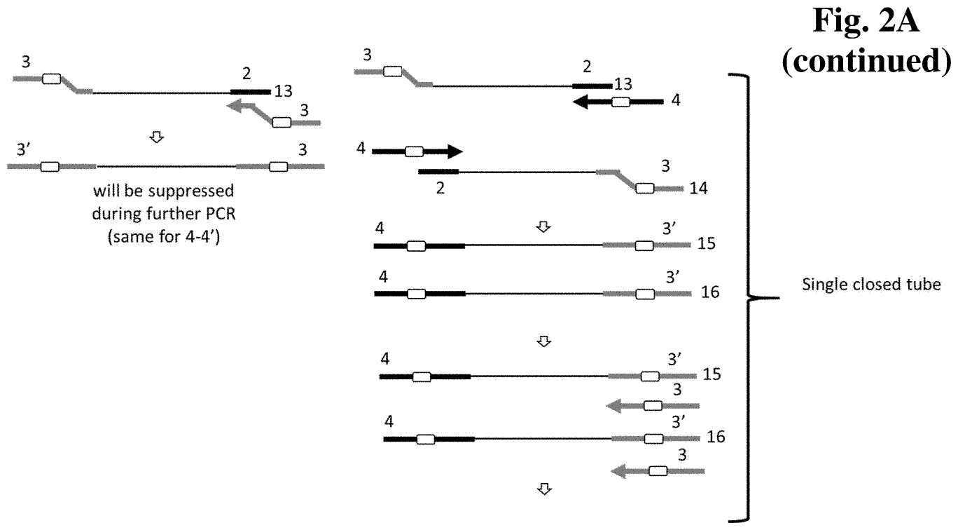

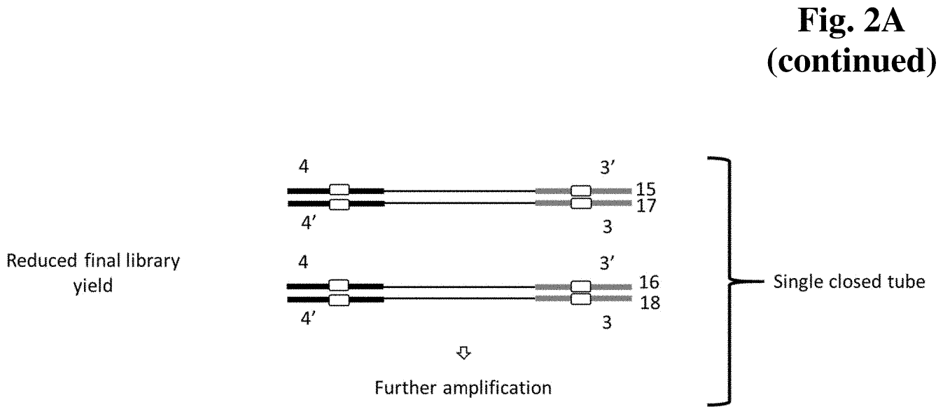

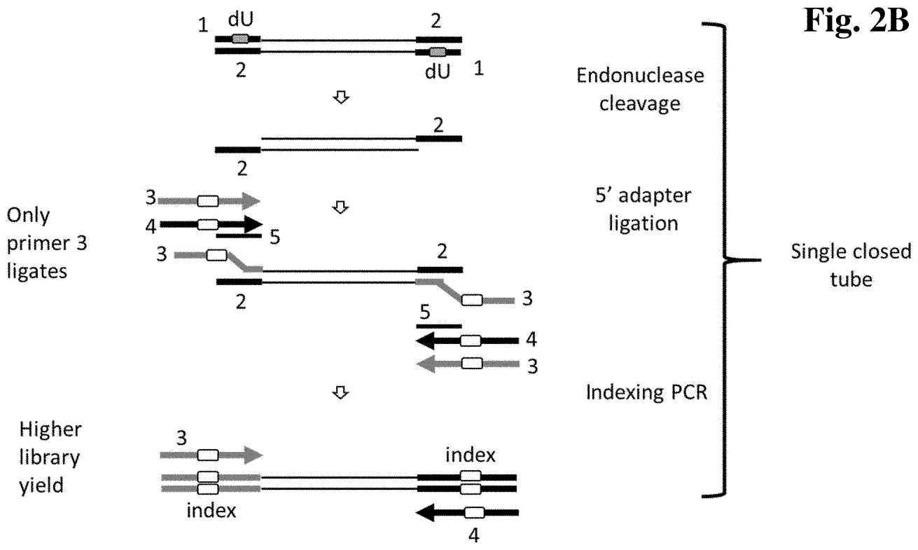

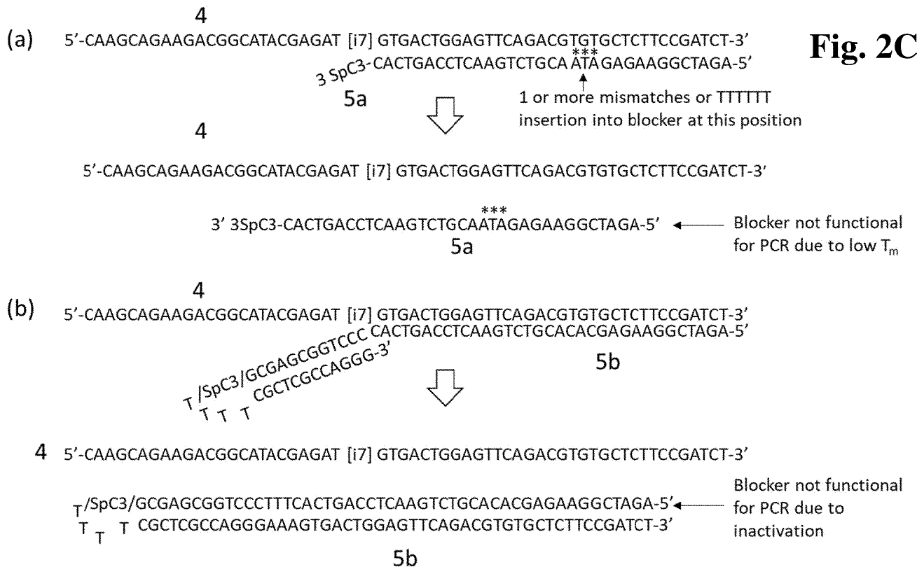

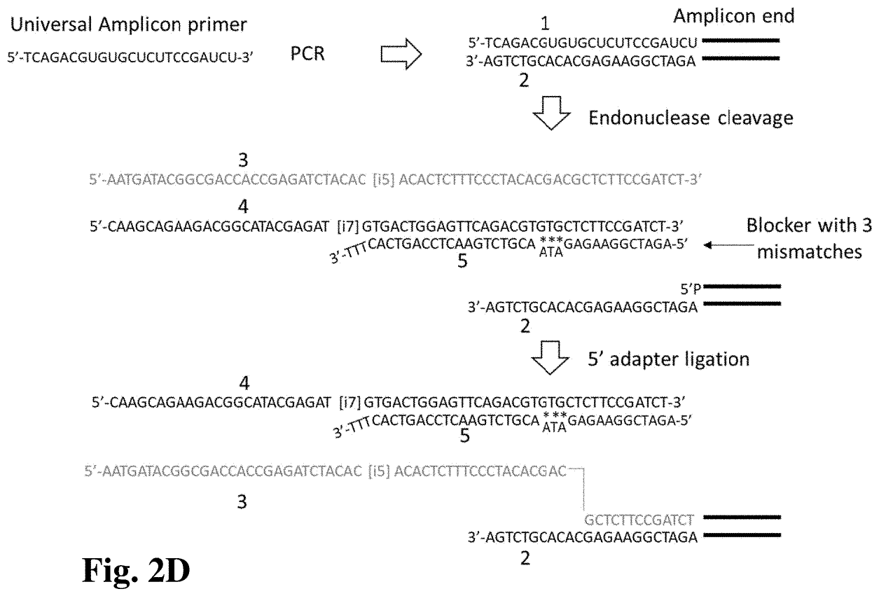

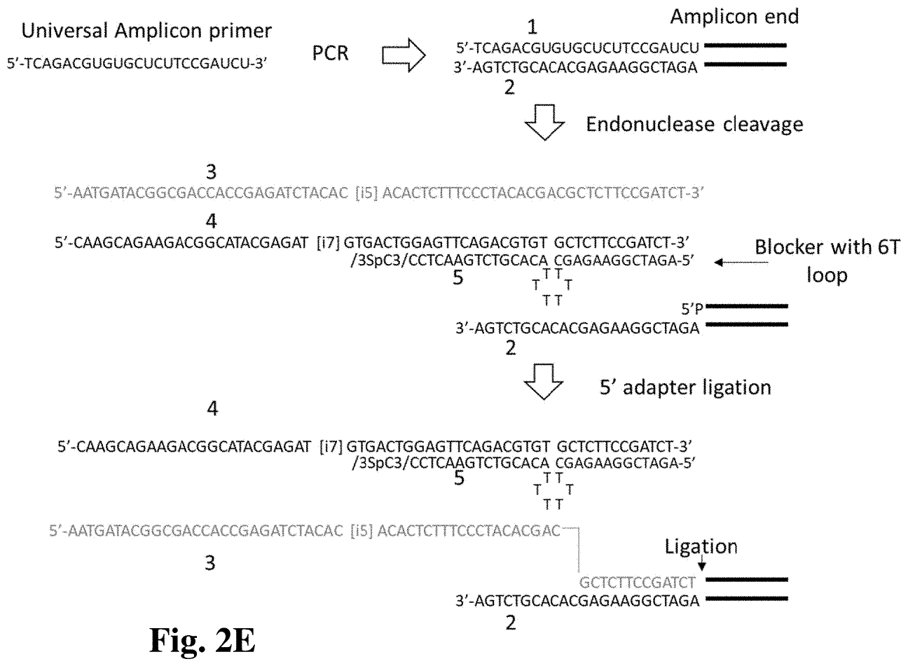

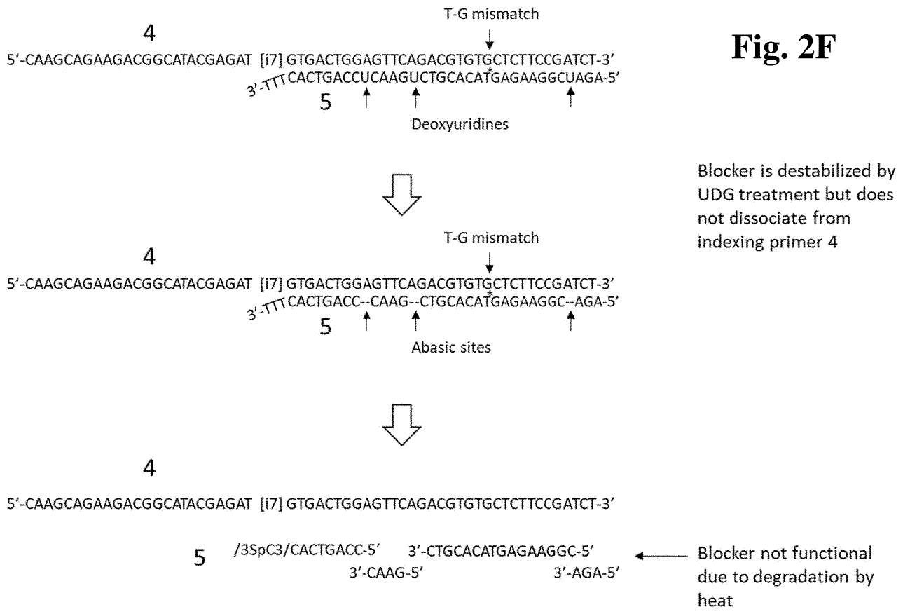

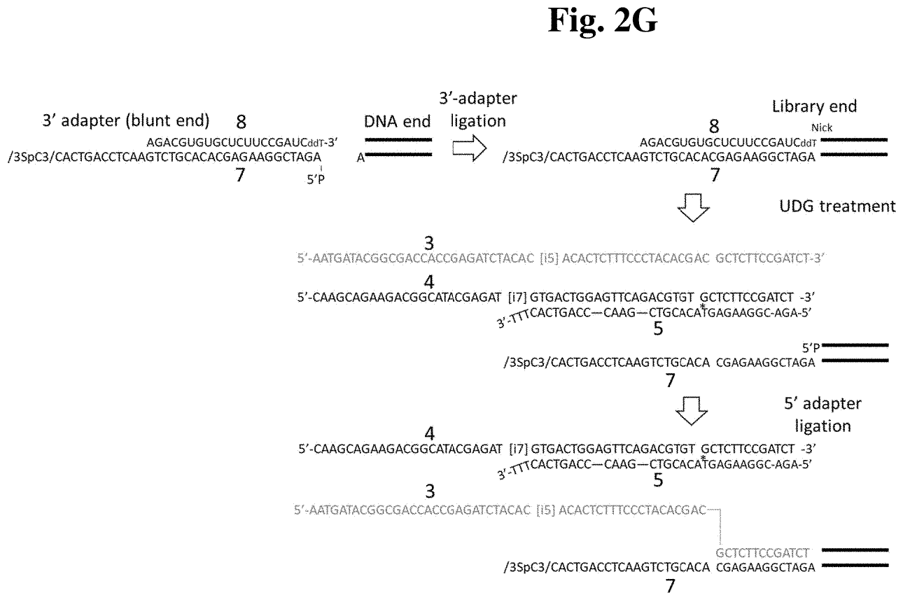

In one embodiment as shown in FIG. 2A, the workflow as presented in FIG. 1A can be performed in the absence of the optional blocker. As shown, the partially double-stranded DNA substrate molecule 10 includes a first strand 11 partially complementary to a second strand 12, where each of the first strand 11 and the second strand 12 form a 3′ overhang 2 at each end of the partially double-stranded DNA substrate molecule 10. With respect to the complementary portion and for the sake of consistency in nomenclature, this complementary portion can be a first portion of the first strand and a third portion of the second strand. It should be understood that, throughout the present disclosure, this first portion of the first strand and the third portion of the second strand refer to the portion as well as the sequence. Thus, a PCR product, although not containing the original first portion could contain the sequence of the first portion and thus would be referred to herein as comprising the first portion. Each overhang also includes a first common nucleotide sequence at the 5′ end of each 3′ overhang 2. Although not intended to be limiting, in FIG. 2A, a double-stranded DNA product can be produced, for example, by multiplex PCR amplification with a universal primer that includes dU bases, to include dU bases from a universal primer 1 which can be cleaved by an endonuclease to yield the partially double-stranded DNA substrate. In such instances, the 3′ overhang 2 can be the reverse complement of the universal primer 1. Because each of the indexing primers 3 (first indexing primer) and 4 (second indexing primer) of the pair have an identical 3′ terminal sequence complementary to the first common nucleotide sequence at the 5′ end of the 3′ overhangs 2 on the substrate amplicons with a Tₘ greater than the ligation reaction temperature, both primers can be utilized in the adapter ligation step. This lack of annealing specificity can reduce formation of functional library molecules that require both the 5′ and 3′ adapters. Only ligation of the 5′ adapter primer 3 (first indexing primer) produces functional library. When 3′ adapter primer 4 (second indexing primer) ligates, a non-functional library with the same adapter at both ends is produced because the universal adapter comprises a truncated 3′ adapter.

As further shown in FIG. 2A, after the ligation step a third strand 13 and a strand oligonucleotide 14 can be obtained which include, in a 5′ to 3′ direction, one of the first indexing primers 3, either the first portion (for the third strand 13) or the third portion (for the fourth strand 14), and the 3′ overhang 2 which includes the first common nucleotide sequence. In a further cycle of PCR, the second indexing primer 4 can anneal to the first common nucleotide sequence of the third strand 13 or fourth strand 14 and be extended by the DNA polymerase to yield a fifth strand 15 and a sixth strand 16, respectively, which include, in a 5′ to 3′ direction, one of the second indexing primers, either the first portion (for the sixth strand 16) or the third portion (for the fifth strand 15), and the reverse complement of the first indexing primer 3′. In a further cycle of PCR, the first indexing primer 3 can anneal to the reverse complement of the first indexing primer 3′ on the fifth strand 15 or the sixth strand 16 and then be extended by the DNA polymerase to yield a seventh strand 17 and an eighth strand 18 which include, in a 5′ to 3′ direction, the first indexing primer 3, the first portion (for the seventh strand 17) or the third portion (for the eighth strand 18) and the reverse complement of the second indexing primer 4′. The seventh strand 17 and the eighth strand 18 are complementary to the fifth strand 15 and sixth strand 16, respectively. Further PCR cycles can allow the first indexing primers and second indexing primers to amplify the double-stranded seventh strand 17 and fifth strand 15 and the double-stranded eighth strand 18 and sixth strand 16 to yield a final library. As further shown in FIG. 2A, in instances where an oligonucleotide is generated that has the first indexing primer on one end and its complement on the other, e.g. when the first indexing primer has ligated and then primed that ligated strand, the complementarity of these ends during PCR will suppress amplification of these undesired products. The same applies when an oligonucleotide is generated that has the second indexing primer on one end and its complement on the other.

In an alternative embodiment, a linear blocker 5 that is pre-annealed to the full-length 3′ adapter indexing primer 4 prior to being added to the ligation reaction is used (FIG. 2B). It prevents the 3′ terminus of this primer from being available to ligate to the amplicon substrates after annealing to sequence 2 at the ligation incubation temperature. However, the blocker Tₘ is lower than the PCR annealing temperature or the blocker is inactivated, so the 3′ adapter indexing primer 4 can efficiently anneal during PCR. FIG. 2C. depicts further details of (a) a linear blocker comprising a low Tₘ so its inactive during PCR or (b) a hairpin blocker that becomes inactivated during PCR when using Illumina TruSeq adapters. An i7 indexing primer 4 and a linear blocker 5a with a lower Tₘ or a hairpin blocker 5b that is inactivated so annealing to the 3′ adapter indexing primer is permitted during the ligation reaction but does not anneal during PCR: (a) the linear blocker 5a comprises one or more mismatches to the universal adapter sequence on indexing primer 4 (3 mismatches are shown) or comprises an insertion of non-complementary bases including but not limited to one or more T deoxynucleotides (6 are shown), either of which reduce its Tₘ below the PCR annealing temperature so that the blocker cannot block priming of the indexing primer. The blocker's mismatches or insertion are located 3′ of the sequence complementary to the common adapter sequence to allow the blocker to anneal to the 3′ end of the primer during the ligation step; the linear blocker also comprises a 3′ C3 spacer blocking group to prevent extension. Regarding (b), the hairpin blocker 5b comprises a stem loop hairpin that enables the 5′ overhang of the blocker to hybridize to the 3′ end of the primer during the ligation step, but upon hot start activation of the polymerase, the hairpin blocker is extended from its 3′ end to create a fully double-stranded hairpin that cannot anneal during PCR due to its stable secondary structure at the PCR annealing temperature. To further illustrate the method using a linear blocker with lower Tₘ due to a mismatch to the adapter sequence, FIG. 2D depicts Illumina TruSeq adapter sequences for aspects of the multiplex PCR, endonuclease cleavage of the incorporated universal primer 1, linear blocker 5 with 3 mismatches pre-annealed to the 3′ adapter indexing primer (i7) 4, and ligation of 3′ end of the 5′ adapter indexing primer (i5) 3 to the 5′ portion of the reverse complement of the universal adapter sequence 2 on the substrate amplicons. In this embodiment, the linear blocker has a non-complementary 3′ tail sequence to prevent extension by a polymerase. To further illustrate the method using a linear blocker with lower Tₘ due to insertion of a 6T deoxynucleotide loop, FIG. 2E. depicts Illumina TruSeq adapter sequences for aspects of the multiplex PCR, endonuclease cleavage of the incorporated universal primer 1, linear blocker 5 with a 6T insertion pre-annealed to the 3′ adapter indexing primer (i7) 4, and ligation of 3′ end of the 5′ adapter indexing primer (i5) 3 to the 5′ end of the substrate amplicons.

FIG. 2F depicts a linear blocker that in addition to a Tₘ reducing T*G mismatch has 3 degradable U bases. Incubation with uracil glycosylase further destabilizes its interaction with the indexing primer 4 by creating 3 abasic sites within the blocker, not sufficient for its dissociation from indexing primer 4 during 5′ adapter indexing primer 3 ligation step, but sufficient for its degradation by heat during PCR. FIG. 2G. depicts Illumina TruSeq adapter sequences for aspects of the blunt end adapter ligation, UDG cleavage and destabilization of the annealed but not covalently attached to DNA 3′ adapter oligonucleotide 8, UDG cleavage and destabilization of linear blocker 5 with a T*G mismatch and 3 uracil nucleotides, pre-annealed to the 3′ adapter indexing primer (i7) 4, and ligation of 3′ end of the 5′ adapter indexing primer (i5) 3 to the 5′ end of the substrate DNA.

Methods Using a Hairpin Adapter

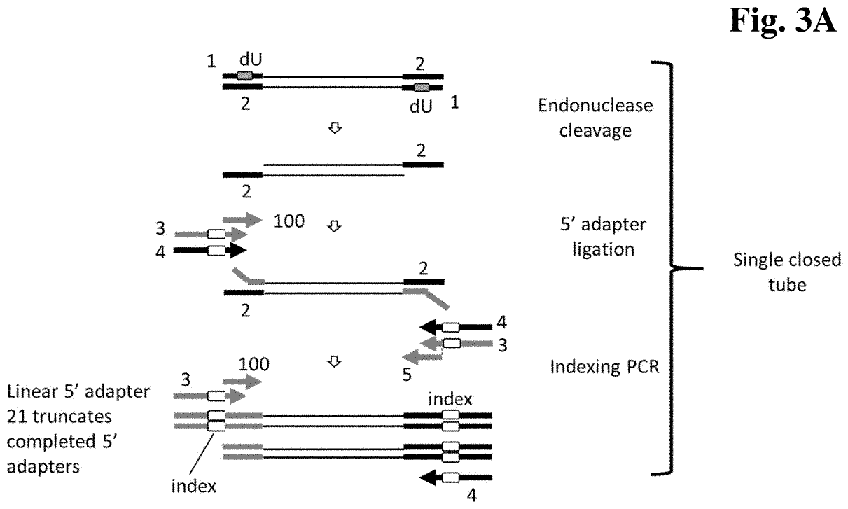

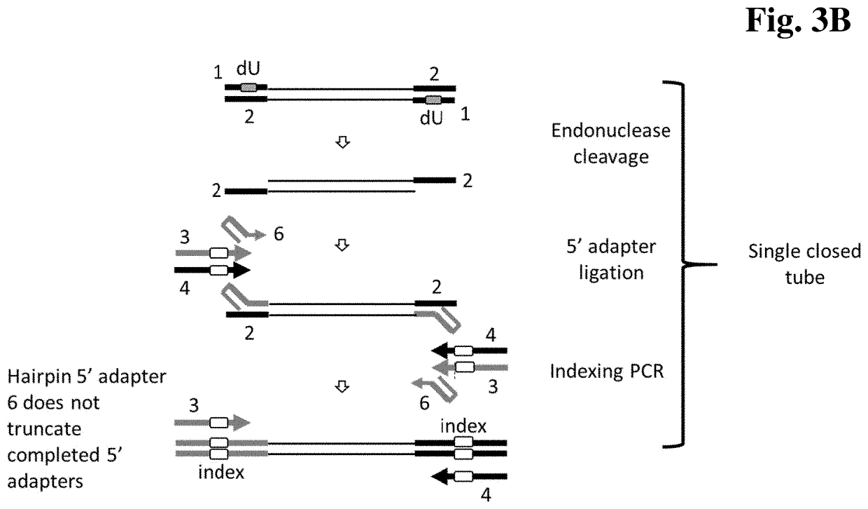

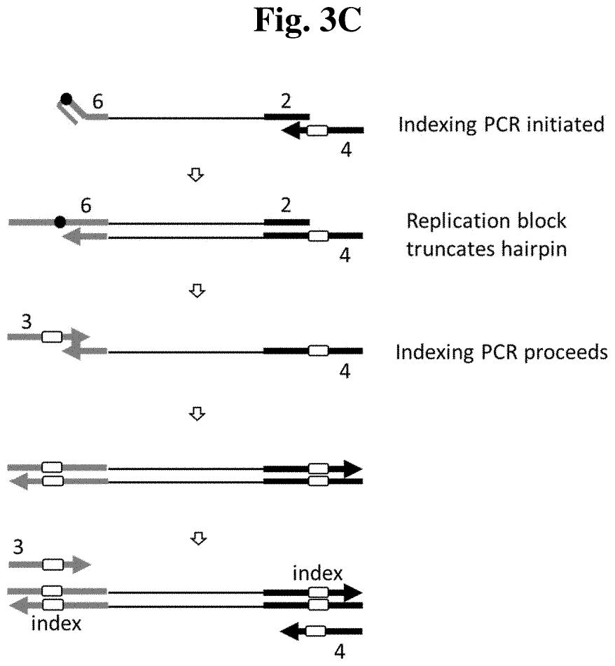

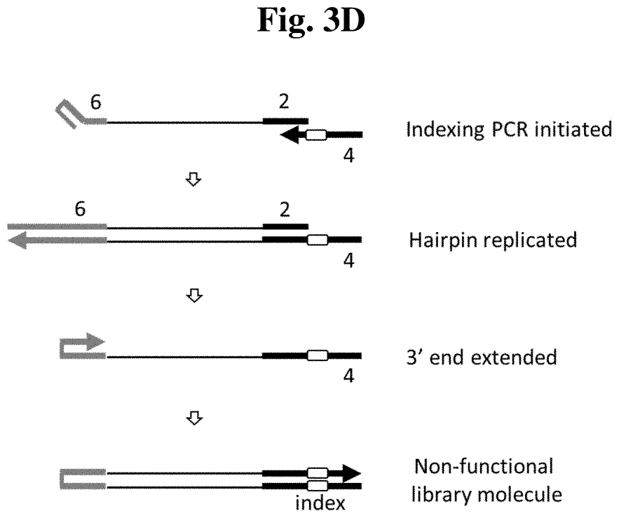

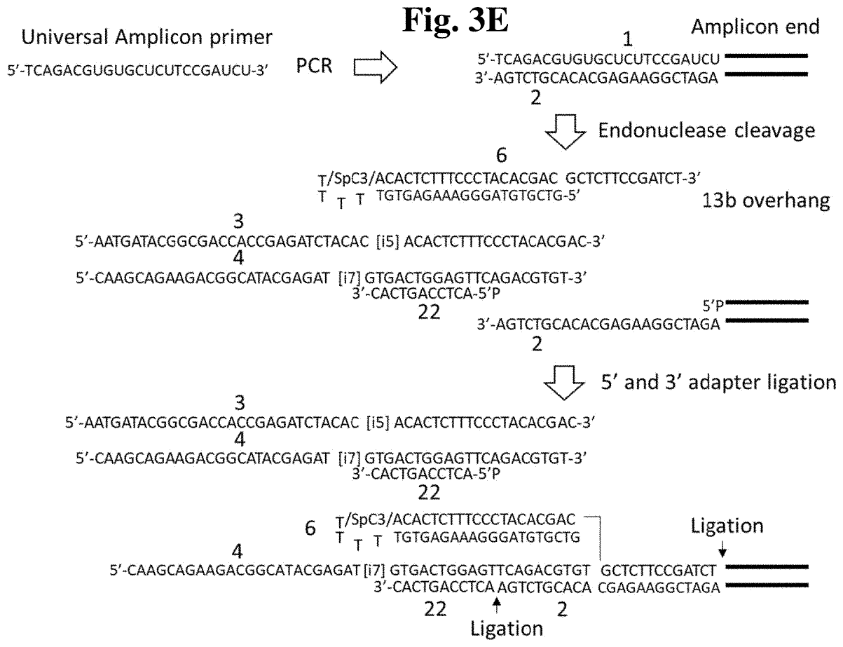

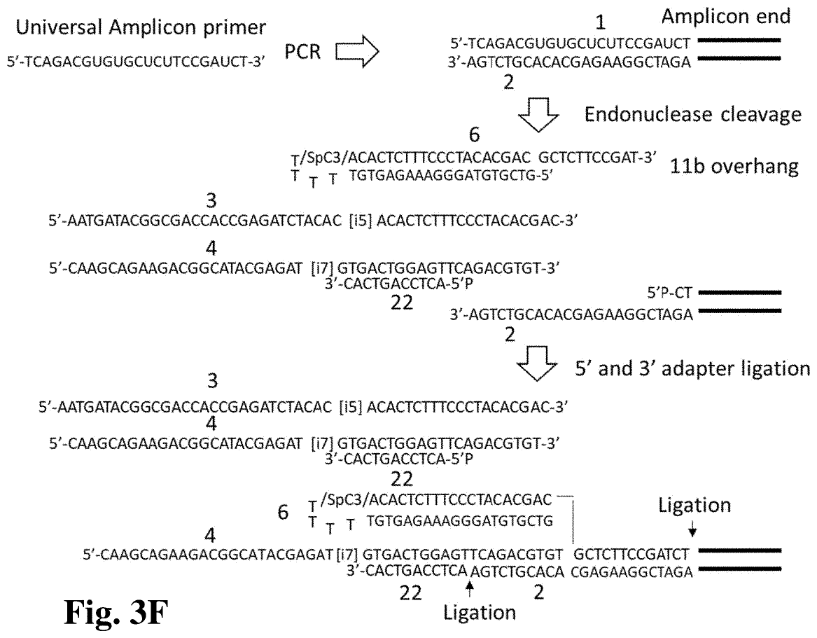

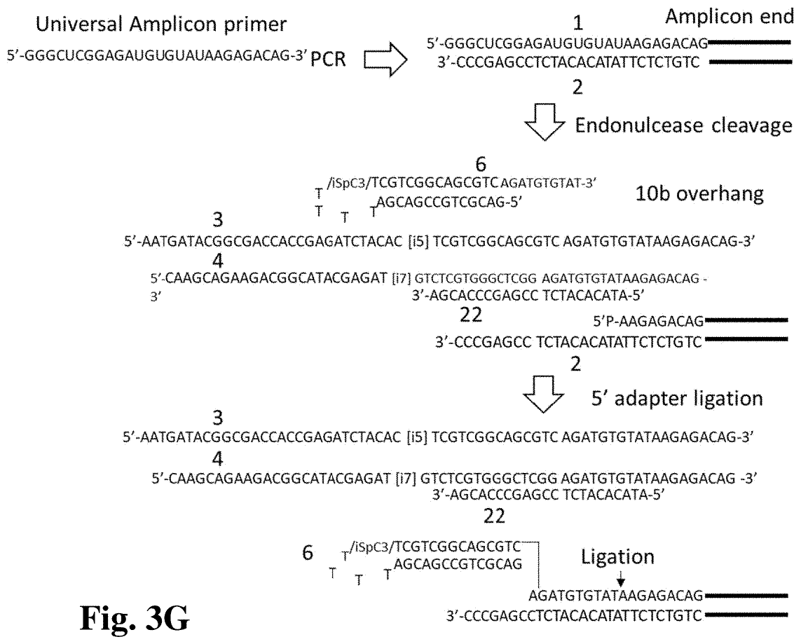

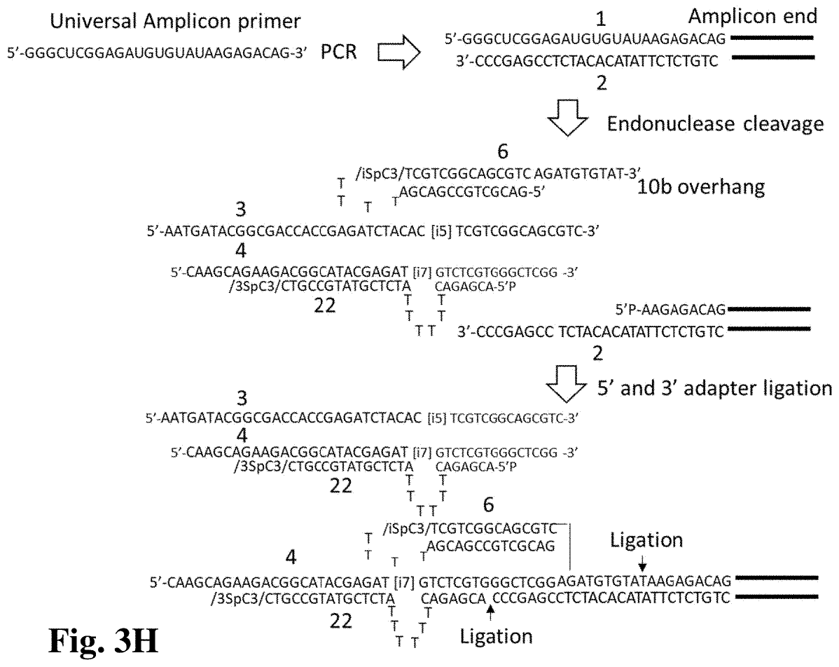

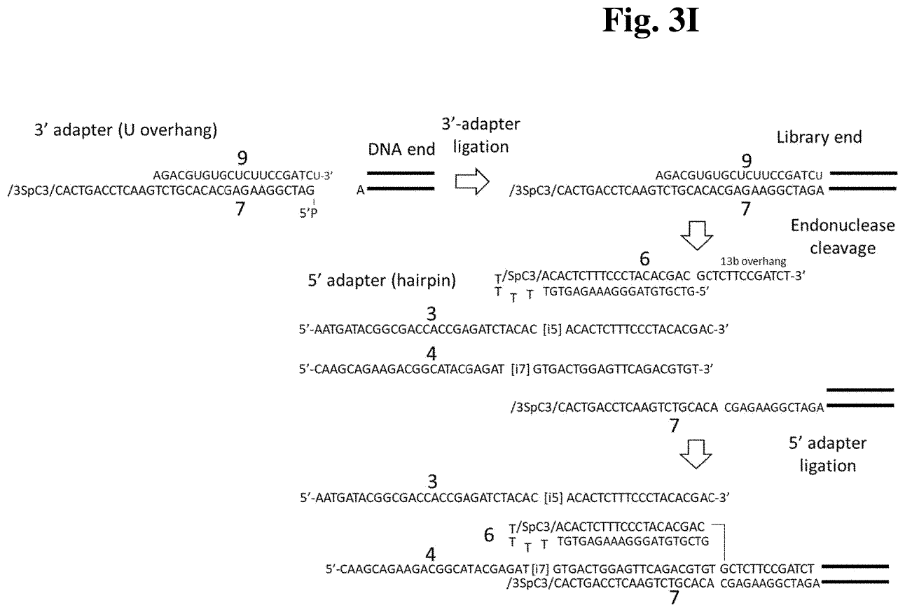

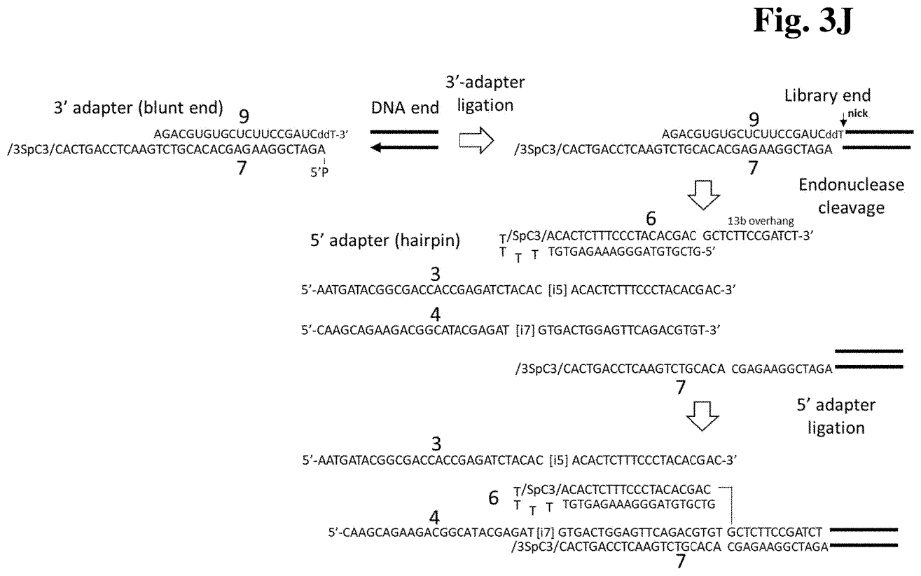

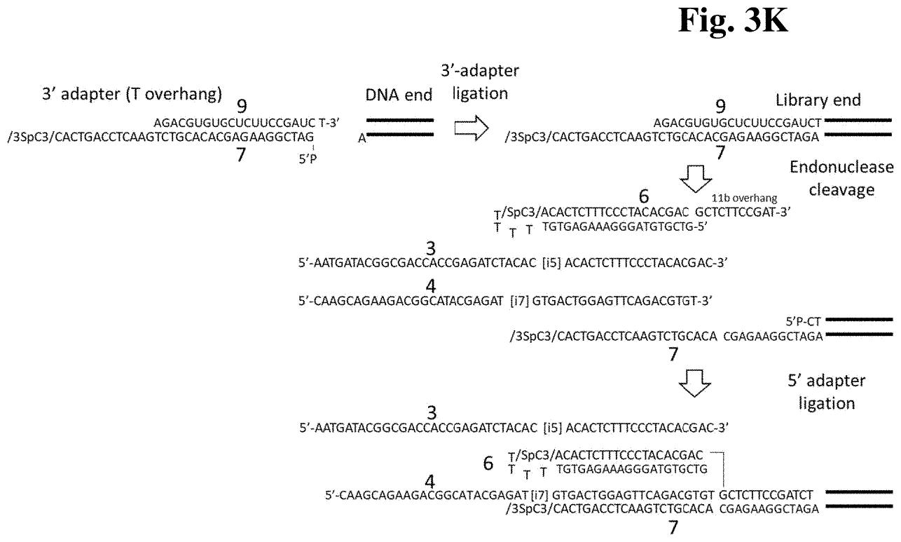

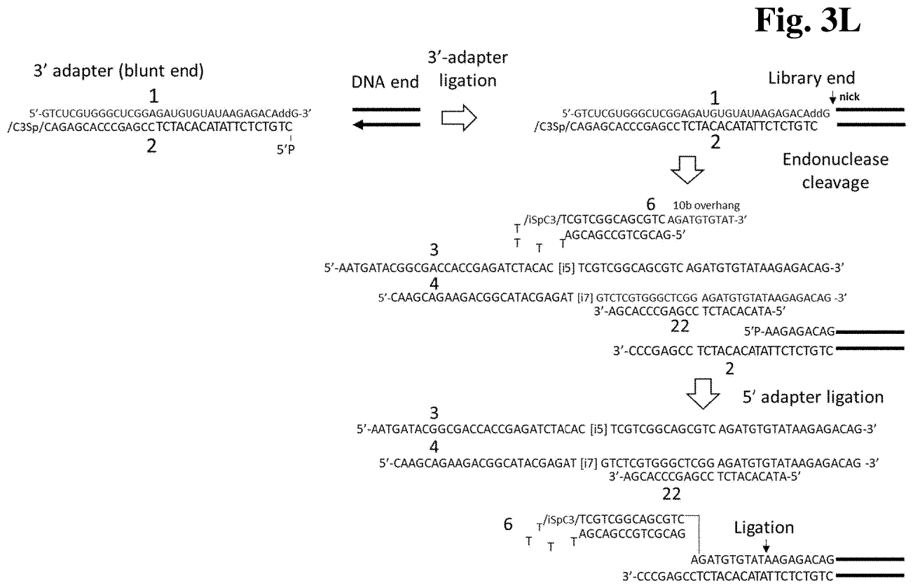

Some alternative embodiments utilize a truncated adapter for ligation-coupled PCR. A truncated adapter is required when using indexing primers that lack the common adapter sequence at their 3′ ends. As shown in FIG. 3A when using a linear truncated adapter 100, the endonuclease cleavage and adapter ligation step proceed efficiently but in order to confer specificity for 5′ adapter indexing primer annealing, the linear truncated 5′ adapter also has a Tₘ sufficient for annealing and extending during the PCR cycles which results in truncation of some completed 5′ adapters and reduced library yield. FIG. 3B. depicts a solution to this problem where a hairpin truncated adapter 5 is used for ligation-coupled-PCR when using indexing primers 3 and 4 that lack the common adapter sequence at the 3′ ends. The endonuclease cleavage is followed by annealing and ligation of the 3′ single-stranded overhang of the hairpin adapter which comprises at least a portion of the common adapter sequence. To confer specificity for subsequent 5′ adapter indexing primer annealing, the hairpin adapter also comprises a unique sequence of the 5′ adapter and the reverse complement of this sequence at the 5′ end of the blocker to create a hairpin also comprising a T loop sequence. This secondary structure reduces its activity as a primer during PCR due to annealing competition with its self-complementarity, and results in higher amplified library yields because the hairpin adapter can not truncate completed adapters during PCR. FIG. 3C. further depicts that the stem loop truncated adapter 6 also comprises a non-replicable modification represented by the black circle within the loop sequence so formation of completely replicated hairpin products are prevented. When indexing PCR is initiated by indexing primer 4, replication of the hairpin is truncated by the replication block. This allows efficient annealing of indexing primer 3 without competition of annealing from the hairpin adapter due to its secondary structure, and efficient indexing PCR. FIG. 3D. depicts the undesired result when the stem loop truncated adapter 6 lacks a non-replicable modification within the loop sequence. In this case, when indexing PCR is initiated by indexing primer 4, replication of the full hairpin occurs. As a result, the 3′ terminus of the replicated hairpin anneals and further extends to generate a fully double-stranded hairpin library molecule which is non-functional. Therefore, use of a non-replication modification is required when using a truncated hairpin adapter.

A specific embodiment using a TruSeq Illumina adapter workflow is presented in FIG. 3E when a hairpin truncated adapter comprising an internal non-replicable C3 spacer is used for ligation-coupled-PCR. In this example, the universal amplicon primer containing dU bases replaces all 6 T deoxynucleotides so the entire primer sequence 1 is cleaved and removed by USER. Therefore, hairpin truncated adapter 6 comprises all 13 bases of the TruSeq common adapter sequence as a 3′ overhang for ligation to the substrate. In addition, because indexing primers 3 and 4 lacking the common adapter sequences at their 3′ ends are used, oligo 6, a portion of the 3′ adapter, is annealed to indexing primer 4 and simultaneously ligated to the 3′ end of sequence 2 on each amplicon in order to provide a sufficient annealing sequence for indexing primer 4 during PCR. FIG. 3F. depicts an alternative embodiment using a TruSeq Illumina adapter workflow where hairpin truncated adapter with internal non-replicable C3 spacer is used for ligation-coupled-PCR. In this example, the universal amplicon primer 1 containing dU bases replaces all T deoxynucleotides except the 3′ most terminal T base, so only a portion of the common adapter sequence is cleaved and removed by USER cleavage. Therefore, hairpin truncated adapter comprises only 11 of the 13 bases of the common adapter sequence as a 3′ overhang for ligation to the substrate. This can reduce bias in endonuclease cleavage by not cleaving the U base adjacent to amplicon ends comprising varied base composition. In addition, because indexing primers 3 and 4 lacking the common adapter sequences at their 3′ ends are used, oligo 22, a portion of the 3′ adapter, is annealed to indexing primer 4 and simultaneously ligated to the 3′ end of sequence 2 on each amplicon in order to provide a sufficient annealing sequence for indexing primer 4 during PCR, as the universal primer comprises a truncated 3′ adapter sequence.

In another alternative embodiment, a Nextera Illumina adapter workflow is used with a hairpin truncated adapter, full-length indexing primers and a linear blocker for ligation-coupled-PCR (FIG. 3G). The Nextera adapters comprise a 19 base sequence common to both adapters. In this example, the universal amplicon primer containing dU bases replaces all 5 T deoxynucleotides, but the 3′ terminal 9 base sequence is retained due to absence of T bases when USER cleavage is performed. Therefore, hairpin truncated adapter 5 comprises the remaining 10 bases of the common adapter sequence as a 3′ overhang, where the 5′ overhang of the hairpin adapter similarly comprises additional sequence unique to the 5′ adapter, its reverse complement and a T loop comprising a non-replicable spacer. In this example, full length Nextera indexing primers 3 and 4 are used but are not compatible for ligation due to the portion of the universal sequence 1 remaining after cleavage by USER. In addition, to prevent indexing primer 4 from competing for annealing and ligation by hairpin adapter 6, linear blocking oligonucleotide 22 is used. In another embodiment, FIG. 3H. depicts another Nextera Illumina adapter workflow where a hairpin truncated adapter is used with indexing primers lacking the common adapter sequence for ligation-coupled-PCR. In this example, the universal amplicon primer 1 containing dU bases replaces all 5 T deoxynucleotides, but the 3′ terminal 9 base sequence is retained due to absence of T bases when USER cleavage is performed. Therefore, hairpin truncated adapter 6 comprises the remaining 10 bases of the common adapter sequence as a 3′ overhang, where the 5′ overhang of the hairpin adapter similarly comprises additional sequence unique to the 5′ adapter, its reverse complement and a T loop comprising a non-replicable spacer. In this example, Nextera indexing primers 3 and 4 lacking the 19 base common adapter sequence at their 3′ ends are utilized, so additionally, oligo 22 comprising a portion of the 3′ adapter, is annealed to indexing primer 4 and simultaneously ligated to the 3′ end of sequence 2 on each amplicon in order to provide a sufficient annealing sequence for indexing primer 4 during PCR. Because truncated adapter 6 must span the sample-specific index sequence to achieve an effective annealing temperature, to generate a universal truncated adapter that will anneal to adapters with different index sequences, the sample-specific index sequence is replaced by a universal T loop.

The next four embodiments illustrate the use of a hairpin 5′ adapter in the ligation-coupled PCR step of the NGS library workflow. Two specific embodiments using a TruSeq Illumina adapter workflow are presented in FIG. 3I and FIG. 3J when a hairpin truncated adapter comprising an internal non-replicable C3 spacer is used for ligation-coupled-PCR. In the example shown in FIG. 31, the 3′ adapter oligonucleotide 9 containing dU bases replaces all 6 T deoxynucleotides so the entire sequence 9 is cleaved and removed by USER. In the example shown in FIG. 3J, due to the absence of a covalent link between the 3′ adapter oligonucleotide 1 and DNA, oligonucleotide 9 Tₘ can be substantially reduced by incubation with UDG and creation of six abasic sites, sufficient for oligonucleotide 9 dissociation from the 3′ adapter oligonucleotide 7 and annealing of the hairpin adapter 6. In both embodiments, hairpin truncated adapter 6 comprises all 13 bases of the TruSeq common adapter sequence as a 3′ overhang for ligation to the substrate.

FIG. 3K. depicts an alternative embodiment using a TruSeq Illumina adapter workflow where hairpin truncated adapter 5 with internal non-replicable C3 spacer is used for ligation-coupled-PCR. In this example, the 3′ adapter oligonucleotide 9 containing dU bases replaces all T deoxynucleotides except the 3′ most terminal T base, so only a portion of the common adapter sequence is cleaved and removed by USER cleavage. Therefore, hairpin truncated adapter 6 comprises only 11 of the 13 bases of the common adapter sequence as a 3′ overhang for ligation to the substrate. This can reduce bias in endonuclease cleavage by not cleaving the U base adjacent to DNA ends comprising varied base composition.

In another alternative embodiment, a Nextera Illumina adapter workflow is used with a hairpin truncated adapter, full-length indexing primers and a linear blocker for ligation-coupled-PCR (FIG. 3L). The Nextera adapters comprise a 19 base sequence common to both adapters. In this example, the 3′ adapter oligonucleotide 9 containing dU bases replaces all 5 T deoxynucleotides, but the 3′ terminal 9 base sequence is retained due to absence of T bases when USER cleavage is performed. Therefore, hairpin truncated adapter 6 comprises the remaining 10 bases of the common adapter sequence as a 3′ overhang, where the 5′ overhang of the hairpin adapter similarly comprises additional sequence unique to the 5′ adapter, its reverse complement and a T loop comprising a non-replicable spacer. In this example, full length Nextera indexing primers 3 and 4 are used but are not compatible for ligation due to the portion of the universal sequence 1 remaining after cleavage by USER. In addition, to prevent indexing primer 4 from competing for annealing and ligation by hairpin adapter 6, linear blocking oligonucleotide 22 is used.

Description of the Blocker

When using full-length indexing primers that each comprise the same common adapter sequence at their 3′ ends, the disclosed blocker oligonucleotide can form a double stranded structure with the 3′ portion of 3′ adapter indexing primer to prevent it from annealing and ligating to the amplicon substrate during 5′ adapter indexing primer ligation. This is because the TruSeq Illumina adapter has a 13 base common adapter sequence (complementary to the first common nucleotide sequence) and the Nextera Illumina adapter has a 19 base common adapter sequence, and the Tₘ of each of these common adapter sequences is significantly higher than thermolabile ligase incubation temperatures, so specificity for annealing and ligating one primer in the presence of both primers is achieved by the blocker. The blocker comprises three criteria for design and function: i) annealing of the blocker to the 3′ adapter indexing primer permits specific ligation of the 5′ adapter indexing primer and formation of a functional NGS library, thus preventing a mixture of ligated products comprising both 5′ and 3′ adapter indexing primers and reduced functional NGS library construction; ii) annealing of the blocker oligonucleotide to the common adapter sequence of the 5′ adapter indexing primer is reduced relative to annealing to the 3′ adapter indexing primer; and iii) the overall blocker Tₘ is below the PCR annealing temperature so it cannot block annealing of the 3′ adapter indexing primer during PCR, or the blocker is inactivated by a polymerase during PCR so that it cannot block annealing of the 3′ adapter indexing primer. This disclosure describes two blocker designs that satisfy these criteria: a linear blocker and a hairpin blocker.

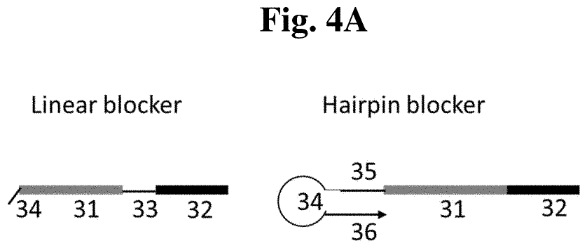

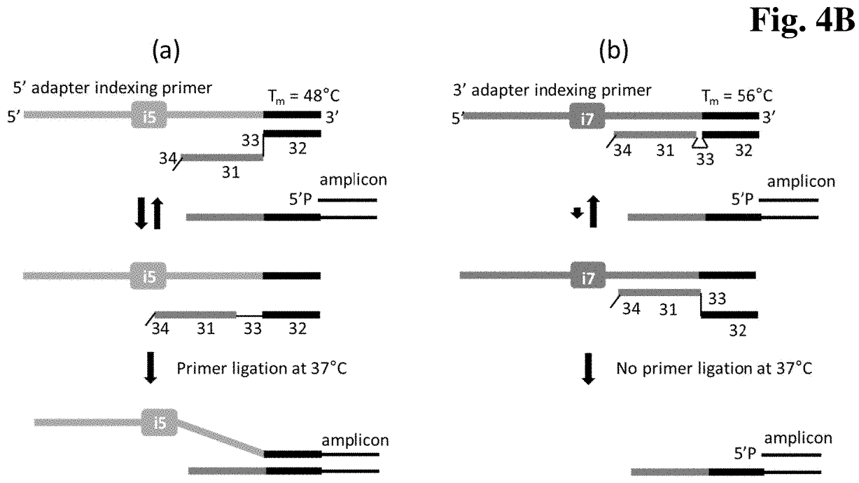

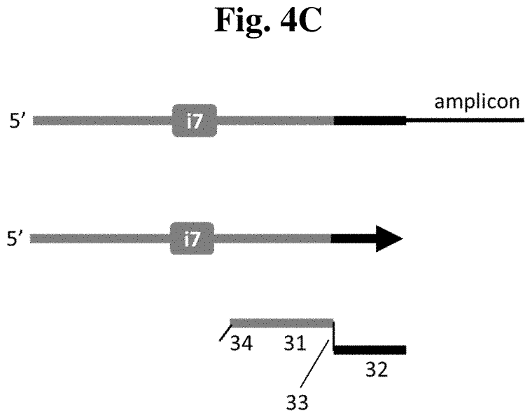

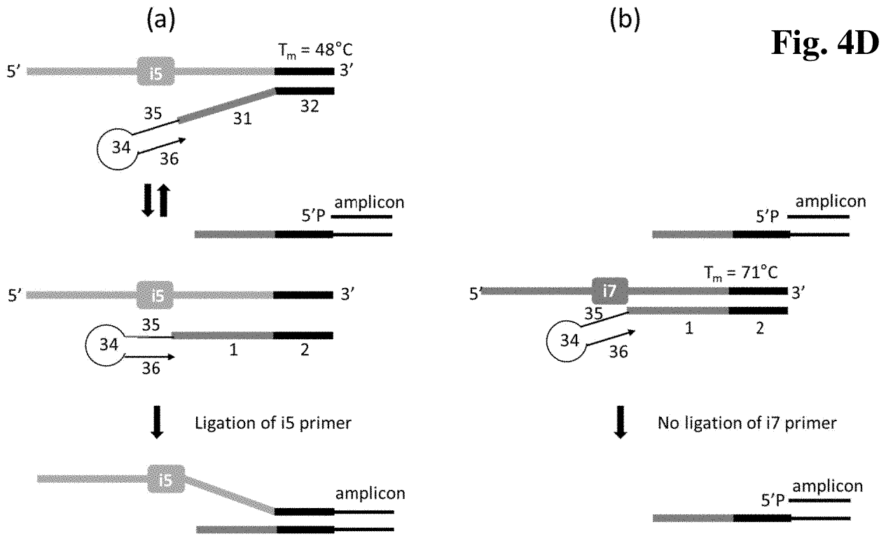

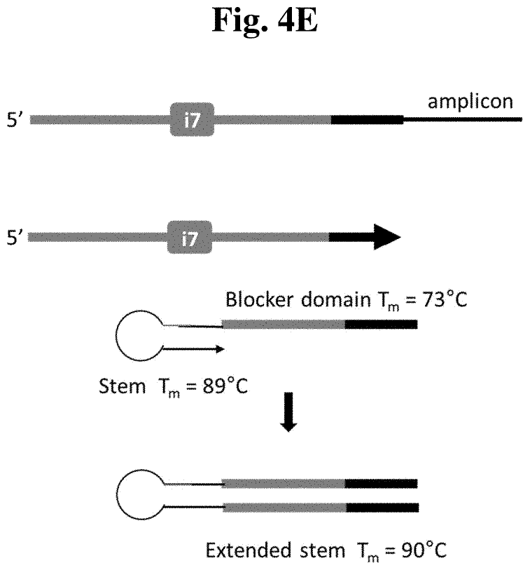

The linear blocker comprises binding domains 31 and 32 where domain 32 is at the 5′ terminus of the blocker, linker domain 33 and a 3′ terminal domain 34 (see FIG. 4A). Binding domain 32 is complementary to at least a portion of the common adapter sequence of both indexing primers (13 bases for TruSeq and 19 bases for Nextera). Binding domain 31 is complementary to at least a portion of the unique adapter sequence positioned 5′ of the common adapter sequence of the 3′ adapter indexing primer but does not include the sample specific index sequence. Binding domain 31 provides greater complementarity to promote selective annealing to the 3′ adapter indexing primer over the 5′ adapter indexing primer. The melting temperature of binding domain 31 should be at least equal or preferably higher than the melting temperature of binding domain 32, and the difference should be at least 1° C. or higher. Linker domain 33 is not complementary to any portion of the indexing primer, thus is a mismatched domain that is used to reduce overall Tₘ and blocker binding efficiency to the 3′ adapter indexing primer during PCR. The linker comprises an insertion of poly T, poly A or poly C sequence or any combination of A, T, C and G bases. Its length can vary from 1 to 50 nucleotides. The linker domain can also comprise a mismatch or a stretch of 2 or more consecutive mismatched nucleotides that are not an insertion of additional nucleotides. The 3′ terminal domain 34 is used to block extension by a polymerase during the PCR phase of the ligation-coupled-PCR. It can comprise a 3′ modification including but not limited to a C3 carbon spacer, hexanediol, spacer 9, spacer 18, phosphate, 2′,3′-dideoxynucleosides ddA, ddT, ddC and ddG, 3′-deoxynucleosides 3′-A, 3′-T, 3′-C and 3′-G, RNA nucleotides such as rU, 3-O-methyl nucleotides, or a DNA sequence that is not complementary to the adjacent primer sequence such as poly T, poly A, poly C and poly G and additionally comprises nuclease resistant linkages to prevent proofreading polymerase 3′-5′ exonuclease activity from removing the DNA sequence that is not complementary to the adjacent primer sequence. As shown in FIG. 4B, binding domain 31 confers specificity for annealing of the blocker to the 3′ adapter indexing primer over the 5′ adapter indexing primer. Although not required, best results are achieved by pre-annealing of the blocker to the 3′ adapter indexing primer prior to adding to the ligation reaction. However, any excess blocker or dissociation of the blocker from the 3′ adapter indexing primer can anneal by domain 32 to the 5′ adapter indexing primer, since the Tₘ of domain 32 is a higher temperature (Tₘ˜48° C.) than the 37° C. incubation temperature. However, due to domain 31, annealing is thermodynamically favored for the 3′ adapter indexing primer FIG. 4B(b) over annealing to the 5′ adapter indexing primer FIG. 4B(a), which results in efficient ligation of the 5′ adapter indexing primer to the amplicon substrate after endonuclease cleavage, and results in no or minimal ligation of the 3′ adapter indexing primer. As shown in FIG. 4C, during PCR, the linear blocker does not anneal to the 3′ adapter indexing primer due to non-complementary domain 33 disrupting the base stacking interaction between domains 31 and 32 and, as a result, overall stability and Tₘ of the blocker, which is lower than the PCR annealing temperature and significantly lower than the competing amplicon substrate that is fully complementary to the 3′ adapter indexing primer.

The alternative hairpin blocker comprises binding domains 31 and 32 similar to the linear blocker, but it lacks a non-complementary linker domain 33 and a blocked 3′ terminus to prevent extension by a polymerase. Instead, the hairpin blocker has a stem-loop structure at its 3′ terminus that is formed by stem domains 35 and 36 and loop domain 34 (FIG. 4D). During the ligation reaction, the hairpin blocker is thermodynamically favored to anneal to the 3′ adapter indexing primer due to complementarity between both domain 31 and 32 of the blocker and primer (Tₘ ˜65-70° C.; FIG. 4D (b)), and where annealing to the 5′ adapter indexing primer is reduced due to decreased complementarity to only domain 32 between the blocker and primer (Tₘ˜48° C.; FIG. 4D(a)). Therefore, ligation specificity for the 5′ adapter indexing primer is achieved in the presence of both indexing primers, even though they have the identical common adapter sequence at their 3′ ends. During the PCR phase of the ligation-coupled-PCR, because it is not blocked, the 3′ end of stem domain 36 of the hairpin blocker is extendable by a DNA polymerase which initiates self-replication of the hairpin blocker oligonucleotide. Self-replication generates a high Tₘ secondary structure ˜90° C. that is maintained at the PCR annealing temperature, thus inactivating the blocker's ability to anneal to and block the 3′ adapter indexing primer, even though the overall Tₘ of domains 31 and 32 is ˜73° C., which is above the PCR annealing temperature (see FIG. 4E). As a result, the 3′ adapter indexing primer can efficiently amplify the template during PCR and is only blocked in the ligation reaction to confer specificity to ligation by the 5′ adapter indexing primer to the amplicon substrate. The length of stem domains 35 and 36 should be sufficient to provide 3′ hairpin stability and priming at the PCR annealing temperature, desirably its melting temperature should be higher than the annealing temperature of both the PCR reaction and blocker domain 31 and 32. In this case, self-replication of the hairpin blocker oligonucleotide can be accomplished at a higher temperature prior to indexing primer annealing that would occur at lower temperature when the blocker has been already inactivated. The length and base composition of loop domain 34 is flexible and comprises 1 to 6 or more T, A, C or G deoxynucleotides, or a combination thereof to allow formation of a stable stem-loop structure by blocker domains 35 and 36 (FIG. 4D).

Additional destabilization of the interaction between the blocker and the 3′ adapter indexing primer can be achieved by replacing one or more T nucleotides within the blocker with dU bases. As a result of incubation with UDG enzyme during 5′ adapter ligation reaction dU bases become excised and create abasic sites that promote fragmentation of the blocker oligonucleotide during the first PCR cycle.

Description of the Hairpin Adapter

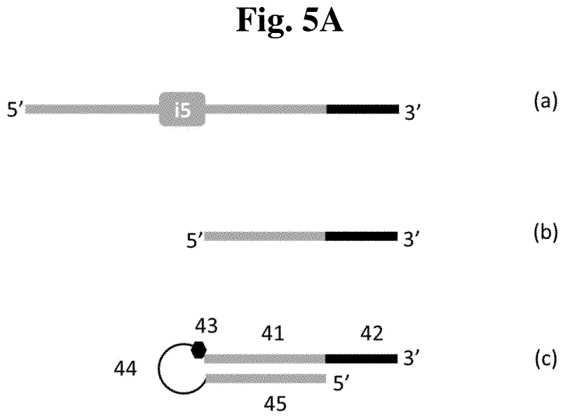

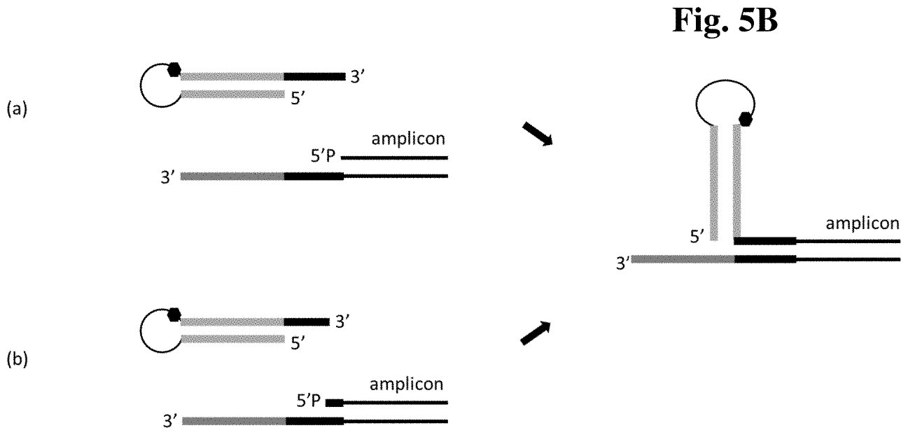

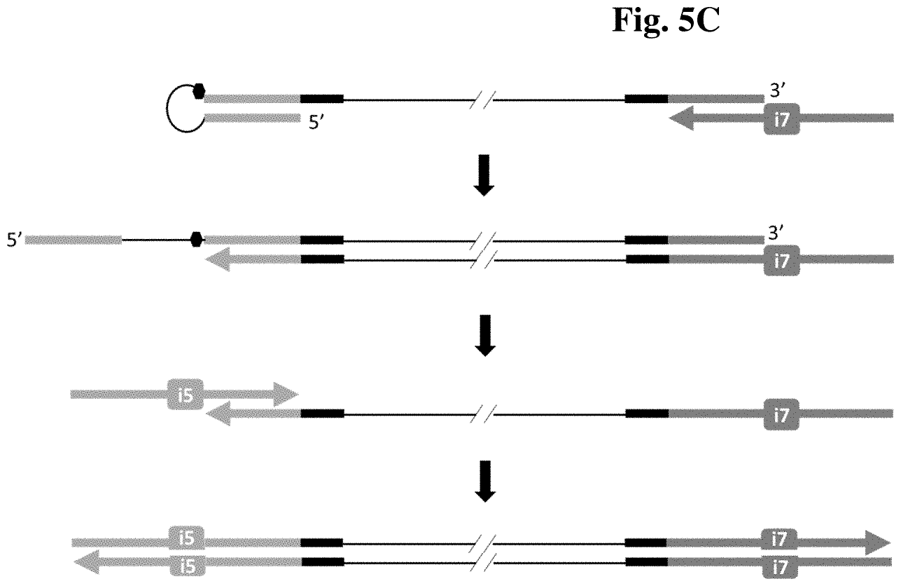

There are two examples when the indexing primer cannot also be used as an adapter for ligation. The first example is when using indexing primers that only comprise unique adapter sequences and lack the common adapter sequence at their 3′ terminus, as they comprise insufficient adapter sequences. In the other example, when endonuclease cleavage of the universal primer on the amplicons does not produce a pattern of cleavage that removes the entire universal primer sequence, leaving a non-digested 3′ portion of the primer at the amplicon junction, the indexing primer 3′ terminus is not compatible for use as a 5′ adapter because it has either insufficient or redundant adapter sequence content at its 3′ end (depending on whether using indexing primers lacking the common adapter sequence or full length indexing primers including the common adapter sequence). For these two embodiments of the method, the ligation-coupled-PCR is supplemented with a truncated 5′ adapter. As shown in FIG. 5A, the truncated 5′ adapter relative to a full length adapter (a) can either be linear (b) or comprise a hairpin with a stem loop structure (c), where the hairpin prevents adapter annealing and extension during PCR due to competition with its stable self-complementarity. This prevents the hairpin adapter from truncating completed 5′ adapters during PCR, making it the preferred embodiment for a truncated 5′ adapter. In contrast, the linear truncated adapter can efficiently anneal, extend, and truncate completed 5′ adapter molecules during PCR which reduces the amplified library yield. Both truncated 5′ adapters both comprise domains 41 and 42 similar to the blocker oligonucleotide although in the reverse complement such that that domain 42 is identical to at least a portion of the common adapter sequence and domain 41 is identical to at least a portion of the unique 5′ adapter sequence 5′ adjacent to the common adapter sequence. Correspondingly, domain 42 is at the 3′ terminus of the truncated adapter. The truncated hairpin adapter shown on FIG. 5A(c) has 5 domains: single stranded domain 42, double stranded domain 41 at least partially complementary to domain 45, replication blocking domain 43, and loop domain 44 where a stem-loop structure is formed. The length and base composition of domain 42 is dictated by the position of the most 3′ cleavable base within the universal primer used in the multiplex PCR step. When endonuclease cleavage of the universal primer occurs at the 3′ terminus of the primer, or at the junction with the amplicon insert (FIG. 5B (a)), domain 42 comprises the entire common adapter sequence (13 bases for the TruSeq adapter and 19 bases for the Nextera adapter). When endonuclease cleavage of the universal primer occurs internally (FIG. 5B(b)), domain 42 is reduced in length to correspond to the remaining 3′ portion of the cleavable primer to restore a contiguous common adapter sequence without introducing overlapping bases or a gap in the sequence, and then the ligase seals the nick. The length and base composition of loop domain 44 is flexible and comprises 1 to 6 or more T, A, C or G deoxynucleotides, or a combination thereof to allow formation of a stable stem-loop structure by adapter domains 41 and 45. During both the ligation and PCR phases of the ligation-coupled-PCR, the truncated hairpin adapter maintains the stem-loop hairpin conformation due to its high stem Tm that is higher than the PCR annealing temperature and as a result does not participate in library amplification as a primer and therefore does not create truncated library products that occurs with the linear truncated adapter. As mentioned previously in the application (FIG. 3C and D), the replication blocking domain 43 prevents replication of the ligated stem-loop structure and formation of non-amplifiable long hairpin structures during PCR. As shown in FIG. 5C, indexing PCR is initiated by the 3′ adapter indexing primer i7, replication stops at the replication blocking group, enabling annealing of the 5′ adapter indexing primer i5 to anneal and extend to complete the indexing PCR cycles.

Method of Primer Assembly

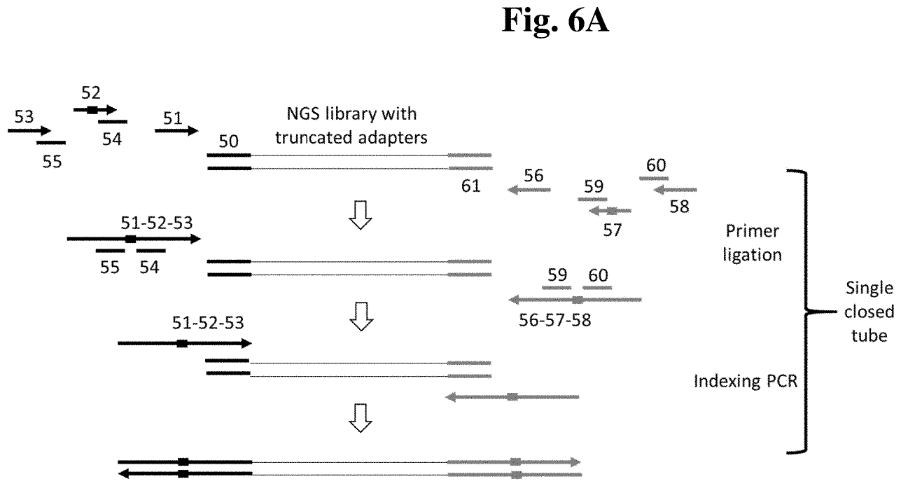

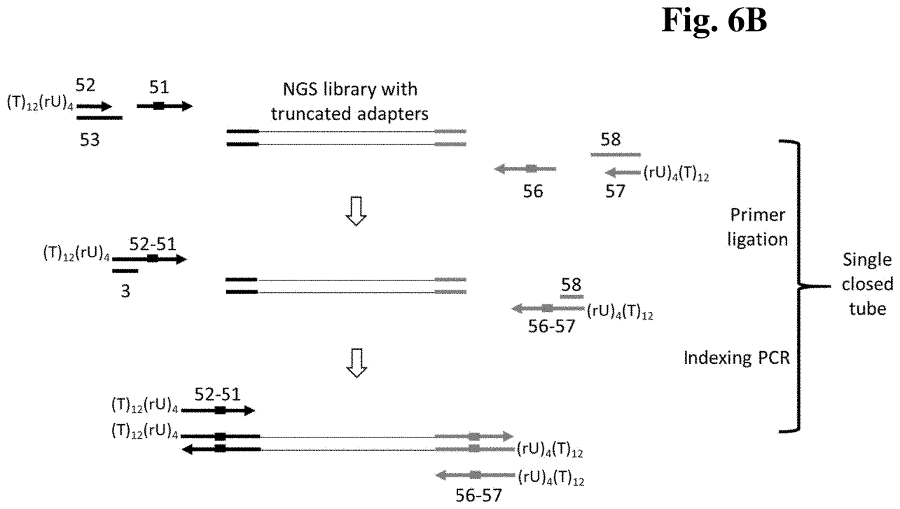

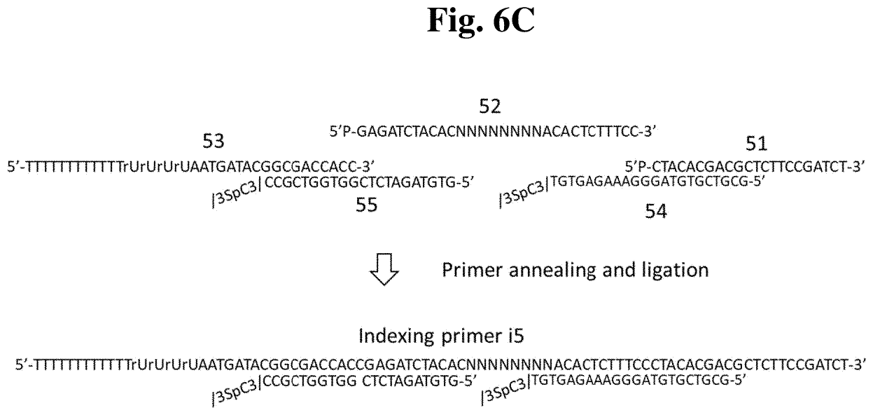

In a different embodiment of ligation-coupled-PCR, a method is disclosed where primer subunits and splints are used to assemble long indexing primers and combined with NGS library amplification. This is used to reduce primer synthesis cost as only short, index-specific oligonucleotide subunits are combined with universal primer subunits instead of synthesizing individual, full-length indexing primers. As shown in FIG. 6A, universal primer subunits 51, 53, 56 and 58 and universal splints 54, 55, 59 and 60 are combined with unique indexing subunits 52 and 57. Primer subunits 51, 52, 56 and 57 comprise a 5′ phosphate for ligation, and the splint oligonucleotides comprise a 3′ blocking group to prevent priming activity during PCR. Annealing and ligation of the primer subunits with the splint oligonucleotides at a first temperature suitable for ligation is followed by thermocycling to amplify the NGS library comprising truncated NGS adapters and complete the adapter sequences and incorporate sample-specific index sequences. FIG. 6B depicts details of a method where Normalase compatible primers are assembled that comprise (T)₁₂(rU)₄ (SEQ ID NO: 1) at their 5′ terminus. 5′ sequences 52 and 57 that are required for downstream enzymatic normalization are splint ligated to indexing primers 51 and 56 with splints 53 and 58. Primers 51 and 56 require a 5′ phosphate for ligation, and splints 53 and 58 have a 3′ blocking group to prevent priming activity during PCR. Annealing and ligation of the primer subunits and splints at a first temperature suitable for ligation is followed by thermocycling to amplify the NGS library comprising truncated NGS adapters and complete the adapter sequences. In a specific embodiment of the method, FIG. 6C. depicts assembly of a TruSeq Illumina indexing primer with the (T)₁₂(rU)₄ (SEQ ID NO: 1) sequence compatible with downstream enzymatic normalization. Indexing primer i5 comprising the 5′ adapter sequence is assembled from three oligonucleotide subunits: the 3′ subunit 51 (22 bases), the intermediate subunit 52 containing index sequence (30 bases), the 5′ subunit 53 containing terminal sequence (T)₁₂(rU)₄ (SEQ ID NO: 1) (34 bases), and two 3′ blocked splint oligonucleotides 54 and 55. To further describe this embodiment, FIG. 6D. depicts a ligation-coupled-PCR workflow when primer assembly of i5 TruSeq Illumina indexing primer of 4C. above is combined with assembly of a corresponding i7 indexing primer comprising the 3′ adapter sequence, annealing of a hairpin blocker to the i7 primer, and ligation of the i5 primer to an amplicon substrate comprising a truncated 3′ adapter (following endonuclease cleavage, not shown).

Description of Enzymes and Reaction Conditions

The ligation-coupled-PCR can include DNA subunits for ligation, a ligase, a polymerase, a primer pair and a substrate for PCR amplification that may be products of the DNA subunit ligation, and optionally an endonuclease. The polymerase can be a thermostable hot-start DNA polymerase such as Taq DNA Polymerase or preferably, for NGS library amplification, the polymerase is a high fidelity polymerase with 3′-5′ exonuclease proofreading activity. When combined with primers for downstream enzymatic normalization comprising a (T)₁₂(rU)₄ 5′ tail sequence (SEQ ID NO: 1), a proofreading polymerase with 3′-5′ exonuclease activity is required to generate a 5′ overhang during PCR. Commercially available enzymes include but are not limited to Kapa HiFi DNA Polymerase (Roche), NEB Q5 DNA Polymerase (NEB), PrimeStar GXL DNA Polymerase (Takara) and High Fidelity DNA Polymerase (Qiagen). PCR reaction conditions are performed as recommended by the polymerase vendor without modification in a 50 uL reaction volume to allow a 20 uL library eluate from a bead-based purification and 5 uL for addition of primers and adapters when using a 2X master mix PCR formulation. The ligase can be any thermolabile ligase capable of high efficiency ligation in a low magnesium PCR reaction buffer including T3 DNA ligase, where a minimum of 30-300 or more enzyme units can be used in a 50 uL ligation-coupled-PCR reaction. The endonuclease for removal of a strand of the first NGS adapter comprising cleavable dU bases is USER enzyme comprising a blend of UDG (Uracil DNA Glycosylase) and Endonuclease VIII (NEB) and 1 enzyme unit is used for a 50 uL ligation-coupled-PCR reaction.

The single, closed tube ligation-coupled-PCR combines all reagents required for ligation and PCR and comprises two separate incubations, a first occurs at a temperature that permits endonuclease and ligase activity but not polymerase activity, and then the reaction is heated to a high temperature to inactivate the endonuclease and ligase, activate the hot start polymerase and denature the substrate for PCR thermocycling. The first reaction is performed with thermolabile enzymes at a temperature between 25 to 37° C., as 25 degrees is optimal for T3 ligase and 37 degrees is optimal for USER enzyme but both can perform within this temperature range. This incubation is ideally performed for 20 minutes to ensure a complete reaction prior to PCR but a range of 5-60 minutes can be performed. The heat inactivation temperature and incubation time is specified by the hot start requirements of the polymerase used in the method, typically 95-98° C. for 30 seconds to 2 minutes or more, and then the annealing and extension temperatures and incubation times are dependent on the primer Tₘ, the polymerase used and the length of the products to be amplified, as known by one of skill in the art, without any specific modification based on the presently disclosed methods.

Within these disclosed methods of ligation-coupled-PCR, indexing primers used solely as primers during PCR or as both adapters during ligation as well as primers during PCR can be used at a reaction concentration of 100 nM each to 1 uM or more each, depending on the number of PCR cycles required. When indexing or other long primers are assembled, splint oligonucleotides are used at a lower molar ratio of 0.75× or up to a 1.5× molar excess over the oligonucleotide subunits, and where primer subunits that are more 5′ in the final primer assembly are at a 2-fold or higher molar concentration than primer subunits that are more 3′ to the final primer assembly. This prevents an excess of non-ligated 3′ primer subunits that would function as primers and truncate the completed adapter during the PCR. An excess of primer subunits at the 5′ end of the final primer assembly do not truncate the final library when they function as individual primers and therefore do not reduce final yield of the PCR product. When a linear or hairpin blocker is utilized, the blocker molar concentration is equal to or greater than the indexing primer concentration it is blocking during the ligation reaction, where the ratio of blocker to primer is 1:1, 1.5:1, 2:1, 4:1, 6:1 or a greater molar ratio. When a hairpin truncated adapter is utilized, it can be used at a reaction concentration of 50-200 nM or more, depending on the quantity of substrate present in the reaction. A similar oligonucleotide concentration of 50-200 nM or more is used when simultaneously ligating an additional portion to the 3′ terminus of the truncated 3′ adapter on the 3′ ends of the amplicon substrates.

Methods for Ligation-Coupled PCR—Both Indexing Primers Include a Common 3′ Terminal Sequence That is Complementary to the First Common Nucleotide Sequence

In some embodiments, a method for ligation-coupled PCR is provided that includes: (i) providing a partially double-stranded DNA substrate comprising a first strand and a second strand, the partially double-stranded DNA substrate including a first 3′ overhang, a double-stranded portion, and a second 3′ overhang, where the first strand includes, in a 5′ to 3′ direction, a first 5′ end, a first portion and a second portion, where the second strand includes, in a 5′ to 3′ direction, a second 5′ end, a third portion and a fourth portion, where the first portion of the first strand and the third portion of the second strand are complementary and form the double-stranded portion, where the second portion of the first strand forms the first 3′ overhang, where the fourth portion of the second strand forms the second 3′ overhang, and where the second portion of the first strand and the fourth portion of the second strand each include a first common nucleotide sequence positioned at a 5′ end of the first 3′ overhang and second 3′ overhang, respectively; (ii) adding a plurality of first indexing primers, a plurality of second indexing primers, a ligase, a DNA polymerase, and dexoxynucleotide triphosphates (dNTPs) to the partially double-stranded DNA substrate to yield a first reaction mixture, where each of the plurality of first indexing primers include a first 3′ terminal portion that is complementary to the first common nucleotide sequence, and where each of the plurality of second indexing primers include a second 3′ terminal portion that is complementary to the first common nucleotide sequence; (iii) incubating the first reaction mixture under a first set of conditions comprising a ligation temperature for a ligation duration, where the first set of conditions is sufficient: 1) for the first 3′ terminal portion of the plurality of first indexing primers to anneal to the first common nucleotide sequence, and 2) for the ligase to ligate one of the plurality of first indexing primers to the first 5′ end of the first strand and one of the plurality of first indexing primers to the second 5′ end of the second strand, to yield a second reaction mixture that includes a third strand which includes, in a 5′ to 3′ direction, one of the plurality of first indexing primers, the first portion, and the second portion, and a fourth strand which includes, in a 5′ to 3′ direction, one of the plurality of first indexing primers, the third portion and the fourth portion; (iv) incubating the second reaction mixture under a second set of conditions that include a first denaturation temperature for a first denaturation duration, a first annealing temperature for a first annealing duration, and a first extension temperature for a first extension duration, sufficient: 1) to inactivate the ligase, denature double-stranded DNA, and, optionally, to activate the DNA polymerase, 2) for the second 3′ terminal portion of one of the plurality of second indexing primers to anneal to the first common nucleotide sequence of the second portion of the third strand one of the plurality of second indexing primers to anneal to the first common nucleotide sequence of the fourth portion of the fourth strand, and 3) for the DNA polymerase to extend the one of the plurality of second indexing primers annealed to the first common nucleotide sequence of the second portion of the third strand and the one of the plurality of second indexing primers annealed to the first common nucleotide sequence of the fourth portion of the fourth strand, to yield a third reaction mixture that includes the third strand, the fourth strand, a fifth strand, and a sixth strand, where the fifth strand includes, in a 5′ to 3′ direction, one of plurality of second indexing primers, the third portion, and a reverse complement of one of the plurality of first indexing primers, and where the sixth strand includes, in a 5′ to 3′ direction, one of the plurality of second indexing primers, the first portion, and the reverse complement of one of the plurality of first indexing primers; (v) incubating the third reaction mixture under a third set of conditions that include a second denaturation temperature for a second denaturation duration, a second annealing temperature for a second annealing duration, and a second extension temperature for a second extension duration, sufficient: 1) to denature double-stranded DNA, 2) for one of the plurality of first indexing primers to anneal to the reverse complement of one of the plurality of first indexing primers of the fifth strand or the sixth strand, and 3) for the DNA polymerase to extent the one of the plurality of first indexing primers annealed to the reverse complement of one of the plurality of first indexing primers of the fifth strand and the one of the plurality of first indexing primers annealed to the reverse complement of one of the plurality of first indexing primers of the sixth strand, to yield a fourth reaction mixture comprising the fifth strand, the sixth strand, a seventh strand, and an eighth strand, where the seventh strand includes, in a 5′ to 3′ direction, one of the plurality of first indexing primers, the first portion, and a reverse complement of one of the plurality of second indexing primers, where the eighth strand includes, in a 5′ to 3′ direction, one of the plurality of first indexing primers, the third portion, and the reverse complement of one of the plurality of second indexing primers, and where the seventh strand is complementary to the fifth strand and the eighth strand is complementary to the sixth strand; and (vi) incubating the fourth reaction mixture under a fourth set of conditions comprising a third denaturation temperature for a third denaturation duration, a third annealing temperature for a third annealing duration, and a third extension temperature for a third extension duration, sufficient for at least a portion of the plurality of first indexing primers and at least a portion of the plurality of second indexing primers to amplify the fifth oligonucleotide and seventh oligonucleotide or the sixth oligonucleotide and eighth oligonucleotide.

In any of the foregoing embodiments, steps (i)-(vi) can be performed in a single closed tube. In any of the foregoing embodiments, the method can not include any purification steps between steps (i)-(vi).

In any of the foregoing embodiments, the partially double-stranded DNA substrate can have a length of about 24 bases to about 6000 bases. By way of example, but not limitation, the partially double-stranded DNA substrate can have a length of about 24 bases to about 6000 bases, about 24 bases to about 5500 bases, about 24 bases to about 5000 bases, about 24 bases to about 4500 bases, about 24 bases to about 4000 bases, about 24 bases to about 3500 bases, about 24 bases to about 3000 bases, about 24 bases to about 2500 bases, about 24 bases to about 2000 bases, about 24 bases to about 1500 bases, about 24 bases to about 1000 bases, about 24 bases to about 750 bases, about 24 bases to about 500 bases, about 24 bases to about 250 bases, about 24 bases to about 200 bases, about 24 bases to about 100 bases, about 24 bases to about 50 bases, about 100 to about 6000 bases, about 100 to about 5500 bases, about 100 to about 5000 bases, about 100 to about 4500 bases, about 100 to about 4000 bases, about 100 to about 3500 bases, about 100 to about 3000 bases, about 100 to about 2500 bases, about 100 to about 2000 bases, about 100 to about 1500 bases, about 100 to about 1000 bases, about 100 to about 750 bases, about 100 to about 500 bases, about 100 to about 250 bases, about 100 to about 200 bases, about 200 bases to about 6000 bases, about 200 baes to about 5500 bases, about 200 bases to about 5000 bases, about 200 to about 4500 bases, about 200 to about 4000 bases, about 200 to about 3500 bases, about 200 to about 3000 bases, about 200 to about 2500 bases, about 200 to about 2000 bases, about 200 to about 1500 bases, about 200 to about 1000 bases, about 200 to about 750 bases, about 200 to about 500 bases, about 200 to about 250 bases, about 250 bases to about 6000 bases, about 250 to about 5500 bases, about 250 to about 5000 bases, about 250 to about 4500 bases, about 250 to about 4000 bases, about 250 to about 3500 bases, about 250 to about 3000 bases, about 250 to about 2500 bases, about 250 bases to about 2000 bases, about 250 to about 1500 bases, about 250 to about 1000 bases, about 250 to about 750 bases, about 250 to about 500 bases, about 500 bases to about 6000 bases, about 500 bases to about 5500 bases, about 500 bases to about 5000 bases, about 500 bases to about 4500 bases, about 500 bases to about 4000 bases, about 500 bases to about 3500 bases, about 500 bases to about 3000 bases, about 500 bases to about 2500 bases, about 500 bases to about 2000 bases, about 500 bases to about 1500 bases, about 500 bases to about 1000 bases, about 500 bases to about 750 bases, about 750 bases to about 6000 bases, about 750 bases to about 5500 bases, about 750 bases to about 5000 bases, about 750 bases to about 4500 bases, about 750 bases to about 4000 bases, about 750 bases to about 3500 bases, about 750 bases to about 3000 bases, about 750 bases to about 2500 bases, about 750 bases to about 2000 bases, about 750 bases to about 1500 bases, about 750 bases to about 1000 bases, about 1000 bases to about 6000 bases, about 1000 bases to about 5500 bases, about 1000 bases to about 5000 bases, about 1000 bases to about 4500 bases, about 1000 bases to about 4000 bases, about 1000 bases to about 3500 bases, about 1000 bases to about 3000 bases, about 1000 bases to about 2500 bases, about 1000 bases to about 2000 bases, about 1000 bases to about 1500 bases, about 1500 bases to about 6000 bases, about 1500 bases to about 5500 bases, about 1500 bases to about 5000 bases, about 1500 bases to about 4500 bases, about 1500 bases to about 4000 bases, about 1500 bases to about 3500 bases, about 1500 bases to about 3000 bases, about 1500 bases to about 2500 bases, about 1500 bases to about 2000 bases, about 2000 bases to about 6000 bases, about 2000 bases to about 5500 bases, about 2000 bases to about 5000 bases, about 2000 bases to about 4500 bases, about 2000 bases to about 4000 bases, about 2000 bases to about 3500 bases, about 2000 bases to about 3000 bases, about 2000 bases to about 2500 bases, about 2500 bases to about 6000 bases, about 2500 bases to about 5500 bases, about 2500 bases to about 5000 bases, about 2500 bases to about 4500 bases, about 2500 bases to about 4000 bases, about 2500 bases to about 3500 bases, about 2500 bases to about 3000 bases, about 3000 bases to about 6000 bases, about 3000 bases to about 5500 bases, about 3000 bases to about 5000 bases, about 3000 bases to about 4500 bases, about 3000 bases to about 4000 bases, about 3000 bases to about 3500 bases, about 3500 bases to about 6000 bases, about 3500 bases to about 5500 bases, about 3500 bases to about 5000 bases, about 3500 to about 4500 bases, about 3500 to about 4000 bases, about 4000 bases to about 6000 bases, about 4000 bases to about 5500 bases, about 4000 bases to about 5000 bases, about 4000 bases to about 4500 bases, about 4500 bases to about 6000 bases, about 4500 bases to about 5500 bases, about 4500 bases to about 5000 bases, about 5000 bases to about 6000 bases, about 5000 bases to about 5500 bases, about 5500 to about 6000 bases, about 24, 30, 40, 50, 60, 70, 80, 90, 100, 200, 250, 500, 750, 1000, 1500, 2000, 2500, 3000, 3500, 4000, 4500, 5000, 5500, or 6000 bases. It should be understood that the length of the partially double-stranded DNA substrate are exemplary and that other sizes are within the scope of the present disclosure. It should be understood that the length of the partially double-stranded DNA substrate refers to the length of the first strand or the second strand of the partially double-stranded DNA substrate, i.e. from the first 5′ end to the 3′ end of the first 3′ overhang or from the second 5′ end to the 3′ end of the second 3′ overhang.

In any of the foregoing embodiments, the first portion and the third portion of the first strand and second strand, respectively, can have a length of about 20 bases to about 6000 bases. By way of example, but not limitation, the first portion of the first oligonucleotide and the third portion of the second oligonucleotide can each have a length of about 20 bases to about 6000 bases, about 20 bases to about 5500 bases, about 20 bases to about 5000 bases, about 20 bases to about 4500 bases, about 20 bases to about 4000 bases, about 20 bases to about 3500 bases, about 20 bases to about 3000 bases, about 20 bases to about 2500 bases, about 20 bases to about 2000 bases, about 20 bases to about 1500 bases, about 20 bases to about 1000 bases, about 20 bases to about 750 bases, about 20 bases to about 500 bases, about 20 bases to about 250 bases, about 20 bases to about 200 bases, about 20 bases to about 100 bases, about 20 bases to about 50 bases, about 100 to about 6000 bases, about 100 to about 5500 bases, about 100 to about 5000 bases, about 100 to about 4500 bases, about 100 to about 4000 bases, about 100 to about 3500 bases, about 100 to about 3000 bases, about 100 to about 2500 bases, about 100 to about 2000 bases, about 100 to about 1500 bases, about 100 to about 1000 bases, about 100 to about 750 bases, about 100 to about 500 bases, about 100 to about 250 bases, about 100 to about 200 bases, about 200 bases to about 6000 bases, about 200 baes to about 5500 bases, about 200 bases to about 5000 bases, about 200 to about 4500 bases, about 200 to about 4000 bases, about 200 to about 3500 bases, about 200 to about 3000 bases, about 200 to about 2500 bases, about 200 to about 2000 bases, about 200 to about 1500 bases, about 200 to about 1000 bases, about 200 to about 750 bases, about 200 to about 500 bases, about 200 to about 250 bases, about 250 bases to about 6000 bases, about 250 to about 5500 bases, about 250 to about 5000 bases, about 250 to about 4500 bases, about 250 to about 4000 bases, about 250 to about 3500 bases, about 250 to about 3000 bases, about 250 to about 2500 bases, about 250 bases to about 2000 bases, about 250 to about 1500 bases, about 250 to about 1000 bases, about 250 to about 750 bases, about 250 to about 500 bases, about 500 bases to about 6000 bases, about 500 bases to about 5500 bases, about 500 bases to about 5000 bases, about 500 bases to about 4500 bases, about 500 bases to about 4000 bases, about 500 bases to about 3500 bases, about 500 bases to about 3000 bases, about 500 bases to about 2500 bases, about 500 bases to about 2000 bases, about 500 bases to about 1500 bases, about 500 bases to about 1000 bases, about 500 bases to about 750 bases, about 750 bases to about 6000 bases, about 750 bases to about 5500 bases, about 750 bases to about 5000 bases, about 750 bases to about 4500 bases, about 750 bases to about 4000 bases, about 750 bases to about 3500 bases, about 750 bases to about 3000 bases, about 750 bases to about 2500 bases, about 750 bases to about 2000 bases, about 750 bases to about 1500 bases, about 750 bases to about 1000 bases, about 1000 bases to about 6000 bases, about 1000 bases to about 5500 bases, about 1000 bases to about 5000 bases, about 1000 bases to about 4500 bases, about 1000 bases to about 4000 bases, about 1000 bases to about 3500 bases, about 1000 bases to about 3000 bases, about 1000 bases to about 2500 bases, about 1000 bases to about 2000 bases, about 1000 bases to about 1500 bases, about 1500 bases to about 6000 bases, about 1500 bases to about 5500 bases, about 1500 bases to about 5000 bases, about 1500 bases to about 4500 bases, about 1500 bases to about 4000 bases, about 1500 bases to about 3500 bases, about 1500 bases to about 3000 bases, about 1500 bases to about 2500 bases, about 1500 bases to about 2000 bases, about 2000 bases to about 6000 bases, about 2000 bases to about 5500 bases, about 2000 bases to about 5000 bases, about 2000 bases to about 4500 bases, about 2000 bases to about 4000 bases, about 2000 bases to about 3500 bases, about 2000 bases to about 3000 bases, about 2000 bases to about 2500 bases, about 2500 bases to about 6000 bases, about 2500 bases to about 5500 bases, about 2500 bases to about 5000 bases, about 2500 bases to about 4500 bases, about 2500 bases to about 4000 bases, about 2500 bases to about 3500 bases, about 2500 bases to about 3000 bases, about 3000 bases to about 6000 bases, about 3000 bases to about 5500 bases, about 3000 bases to about 5000 bases, about 3000 bases to about 4500 bases, about 3000 bases to about 4000 bases, about 3000 bases to about 3500 bases, about 3500 bases to about 6000 bases, about 3500 bases to about 5500 bases, about 3500 bases to about 5000 bases, about 3500 to about 4500 bases, about 3500 to about 4000 bases, about 4000 bases to about 6000 bases, about 4000 bases to about 5500 bases, about 4000 bases to about 5000 bases, about 4000 bases to about 4500 bases, about 4500 bases to about 6000 bases, about 4500 bases to about 5500 bases, about 4500 bases to about 5000 bases, about 5000 bases to about 6000 bases, about 5000 bases to about 5500 bases, about 5500 to about 6000 bases, about 24, 30, 40, 50, 60, 70, 80, 90, 100, 200, 250, 500, 750, 1000, 1500, 2000, 2500, 3000, 3500, 4000, 4500, 5000, 5500, or 6000 bases. It should be understood that the length of the first portion of the first strand and the third portion of the second strand are exemplary and that other sizes are within the scope of the present disclosure.

In any of the foregoing embodiments, the second portion of the first strand and the fourth portion of the second strand, i.e. the first 3′ overhang and the second 3′ overhang, respectively, can each include from about 4 bases to about 100 bases. By way of example, but not limitation, the second portion of the first strand and the fourth portion of the second strand can each include from about 4 bases to about 100 bases, about 4 bases to about 90 bases, about 4 bases to about 80 bases, about 4 bases to about 75 bases, about 4 bases to about 70 bases, about 4 bases to about 60 bases, about 4 bases to about 50 bases, about 4 bases to about 40 bases, about 4 bases to about 30 bases, about 4 bases to about 25 bases, about 4 bases to about 20 bases, about 4 bases to about 15 bases, about 4 bases to about 10 bases, about 4 bases to about 5 bases, about 5 bases to about 100 bases, about 5 bases to about 90 bases, about 5 bases to about 80 bases, about 5 bases to about 75 bases, about 5 bases to about 70 bases, about 5 bases to about 60 bases, about 5 bases to about 55 bases, about 5 bases to about 50 bases, about 5 bases to about 40 bases, about 5 bases to about 30 bases, about 5 bases to about 25 bases, about 5 bases to about 20 bases, about 5 bases to about 15 bases, about 5 bases to about 10 bases, about 10 bases to about 100 bases, about 10 bases to about 90 bases, about 10 bases to about 80 bases, about 10 bases to about 75 bases, about 10 bases to about 70 bases, about 10 bases to about 60 bases, about 10 bases to about 50 bases, about 10 bases to about 40 bases, about 10 bases to about 30 bases, about 10 bases to about 25 bases, about 10 bases to about 20 bases, about 10 bases to about 15 bases, about 15 bases to about 100 bases, about 15 bases to about 90 bases, about 15 bases to about 80 bases, about 15 bases to about 75 bases, about 15 bases to about 70 bases, about 15 bases to about 60 bases, about 15 bases to about 50 bases, about 15 bases to about 40 bases, about 15 bases to about 30 bases, about 15 bases to about 25 bases, about 15 bases to about 20 bases, about 20 bases to about 100 bases, about 20 bases to about 90 bases, about 20 bases to about 80 bases, about 20 bases to about 75 bases, about 20 bases to about 70 bases, about 20 bases to about 60 bases, about 20 bases to about 50 bases, about 20 bases to about 40 bases, about 20 bases to about 30 bases, about 20 bases to about 25 bases, about 25 bases to about 100 bases, about 25 bases to about 90 bases, about 25 bases to about 80 bases, about 25 bases to about 75 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 75 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 75 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 75 bases, about 50 bases to about 70 bases, about 50 bases to about 60 bases, about 60 bases to about 100 bases, about 60 bases to about 90 bases, about 60 bases to about 80 bases, about 60 bases to about 75 bases, about 60 bases to about 70 bases, about 70 bases to about 100 bases, about 70 bases to about 90 bases, about 70 bases to about 80 bases, about 70 bases to about 75 bases, about 75 bases to about 100 bases, about 75 bases to about 90 bases, about 75 bases to about 80 bases, about 80 bases to about 100 bases, about 80 bases to about 90 bases, about 90 bases to about 100 bases, about 4, 5, 6, 7, 8, 9, 10, 11, 12, 13, 14, 15, 20, 25, 30, 33, 40, 50, 60, 70, 75, 80, 90, or 100 bases.

In any of the foregoing embodiments, the first common nucleotide sequence can include from about 1 base to about 50 bases. By way of example, but not limitation, the first common nucleotide sequence can include from about 1 base to about 50 bases, about 1 base to about 45 bases, about 1 base to about 40 bases, about 1 base to about 35 bases, about 1 base to about 30 bases, about 1 base to about 25 bases, about 1 base to about 20 bases, about 1 base to about 15 bases, about 1 base to about 10 bases, about 5 to about 50 bases, about 5 bases to about 45 bases, about 5 bases to about 40 bases, about 5 bases to about 35 bases, about 5 bases to about 30 bases, about 5 bases to about 25 bases, about 5 bases to about 20 bases, about 5 bases to about 15 bases, about 5 bases to about 10 bases, about 10 bases to about 50 bases, about 10 bases to about 45 bases, about 10 bases to about 40 bases, about 10 bases to about 35 bases, about 10 bases to about 30 bases, about 10 bases to about 25 bases, about 10 bases to about 20 bases, about 10 bases to about 15 bases, about 15 bases to about 50 bases, about 15 bases to about 45 bases, about 15 bases to about 40 bases, about 15 bases to about 35 bases, about 15 bases to about 30 bases, about 15 bases to about 25 bases, about 15 bases to about 20 bases, about 20 bases to about 50 bases, about 20 bases to about 45 bases, about 20 bases to about 40 bases, about 20 bases to about 35 bases, about 20 bases to about 30 bases, about 25 bases to about 30 bases, about 30 bases to about 50 bases, about 30 bases to about 45 bases, about 30 bases to about 40 bases, about 30 bases to about 35 bases, about 35 bases to about 50 bases, about 35 bases to about 45 bases, about 35 bases to about 40 bases, about 40 bases to about 50 bases, about 40 bases to about 45 bases, about 45 bases to about 50 bases, about 1, 2, 3, 4, 5, 6, 7, 8, 9, 10, 11, 12, 13, 14, 15, 20, 25, 30, 33, 35, 40, or 50 bases. Preferably, the first common nucleotide sequence comprises 13 bases. In some embodiments, the first common nucleotide sequence includes the sequence of SEQ ID NO: 127 (5′-AGATCGGAAGAGC-3′). Where the first common nucleotide sequence includes the sequence of SEQ ID NO: 127, the first 3′ terminal portion and the second 3′ terminal portion can each include the sequence of SEQ ID NO: 126 (5′-GCTCTTCCGATCT-3′).

In any of the foregoing embodiments, the second portion of the first strand and the fourth portion of the second strand can each further include a second common nucleotide sequence positioned 3′ to the first common nucleotide sequence. In certain aspects, the second common nucleotide sequence can have a length from about 3 bases to about 100 bases. By way of example, but not limitation, the second common nucleotide sequence can have a length from about 1 base to about 100 bases, about 1 base to about 90 bases, about 1 base to about 80 bases, about 1 base to about 75 bases, about 1 base to about 70 bases, about 1 base to about 60 bases, about 1 base to about 50 bases, about 1 base to about 40 bases, about 1 base to about 30 bases, about 1 base to about 25 bases, about 1 base to about 20 bases, about 1 base to about 15 bases, about 1 base to about 10 bases, about 1 base to about 5 bases, about 5 bases to about 100 bases, about 5 bases to about 90 bases, about 5 bases to about 80 bases, about 5 bases to about 75 bases, about 5 bases to about 70 bases, about 5 bases to about 60 bases, about 5 bases to about 50 bases, about 5 bases to about 40 bases, about 5 bases to about 30 bases, about 5 bases to about 25 bases, about 5 bases to about 20 bases, about 5 bases to about 15 bases, about 5 bases to about 10 bases, about 10 bases to about 100 bases, about 10 bases to about 90 bases, about 10 bases to about 80 bases, about 10 bases to about 75 bases, about 10 bases to about 70 bases, about 10 bases to about 60 bases, about 10 bases to about 50 bases, about 10 bases to about 40 bases, about 10 bases to about 30 bases, about 10 bases to about 25 bases, about 10 bases to about 20 bases, about 10 bases to about 15 bases, about 15 bases to about 100 bases, about 15 bases to about 90 bases, about 15 bases to about 80 bases, about 15 bases to about 75 bases, about 15 bases to about 70 bases, about 15 bases to about 60 bases, about 15 bases to about 50 bases, about 15 bases to about 40 bases, about 15 bases to about 30 bases, about 15 bases to about 25 bases, about 15 bases to about 20 bases, about 20 bases to about 100 bases, about 20 bases to about 90 bases, about 20 bases to about 80 bases, about 20 bases to about 75 bases, about 20 bases to about 70 bases, about 20 bases to about 60 bases, about 20 bases to about 50 bases, about 20 bases to about 40 bases, about 20 bases to about 30 bases, about 20 bases to about 25 bases, about 25 bases to about 100 bases, about 25 bases to about 90 bases, about 25 bases to about 80 bases, about 25 bases to about 75 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 75 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 75 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 75 bases, about 50 bases to about 70 bases, about 50 bases to about 60 bases, about 60 bases to about 100 bases, about 60 bases to about 90 bases, about 60 bases to about 80 bases, about 60 bases to about 75 bases, about 60 baes to about 70 bases, about 70 bases to about 100 bases, about 70 bases to about 90 bases, about 70 bases to about 80 bases, about 70 bases to about 75 bases, about 75 bases to about 100 bases, about 75 bases to about 90 bases, about 75 bases to about 80 bases, about 80 bases to about 100 bases, about 80 bases to about 90 bases, about 90 bases to about 100 bases, about 1, 2, 3, 4, 5, 6, 7, 8, 9, 10, 15, 20, 25, 30, 40, 50, 60, 75, 80, 90, or 100 bases.

In embodiments where the second portion of the first strand and the fourth portion of the second strand further include the second common nucleotide sequence, each of the plurality of second indexing primers can include a first 5′ portion positioned 5′ to the second 3′ terminal portion and complementary to the second common nucleotide sequence. In such embodiments, the second set of conditions in step (iv)(b) can be sufficient for the second 3′ terminal portion and the first 5′ portion of one of the plurality of second indexing primers to anneal to at least the first common nucleotide sequence and the second common nucleotide sequence on the second portion of the third strand or the fourth portion of the fourth strand. In such embodiments, a melting temperature of the first 3′ terminal sequence to the first common nucleotide sequence can be lower than the first annealing temperature. By way of further example, but not limitation, the melting temperature of the first 3′ terminal sequence to the first common nucleotide sequence can be lower than the first annealing temperature, the second annealing temperature, and the third annealing temperature. In such embodiments, each of the first indexing primers can not include a sequence complementary to the second common nucleotide sequence. In such embodiments, a melting temperature between the first common nucleotide sequence and second common nucleotide sequence and the second 3′ terminal portion and first 5′ portion of each of the second indexing primers can be greater than the first annealing temperature. In such embodiments, the melting temperature of the first common nucleotide sequence and the second common nucleotide sequence to each of the plurality of second indexing primers can be greater than the first annealing temperature and the third annealing temperature.

In any of the foregoing embodiments, a melting temperature between each of the plurality of first indexing primers and the second portion or fourth portion is less than a melting temperature between each of the plurality of second indexing primers and the second or fourth portion.

In any of the foregoing embodiments, each of the plurality of first indexing primers can have a length of from about 20 bases to about 100 bases. By way of example, but not limitation, each of the plurality of first indexing primers can have a length from about 20 bases to about 100 bases, about 20 bases to about 90 bases, about 20 bases to about 80 bases, about 20 bases to about 70 bases, about 20 bases to about 60 bases, about 20 bases to about 50 bases, about 20 bases to about 40 bases, about 20 bases to about 30 bases, about 25 bases to about 100 bases, about 25 bases to about 90 bases, about 25 bases to about 80 bases, about 25 bases to about 70 bases, about 25 bases to about 60 bases, about 25 bases to about 50 bases, about 25 bases to about 40 bases, about 25 bases to about 30 bases, 30 bases to about 100 bases, about 30 bases to about 90 bases, about 30 bases to about 80 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 70 bases, about 50 bases to about 60 bases, about 60 bases to about 100 bases, about 60 bases to about 90 bases, about 60 bases to about 80 bases, about 60 bases to about 70 bases, about 70 bases to about 100 bases, about 70 bases to about 90 bases, about 70 bases to about 80 bases, about 80 bases to about 100 bases, about 80 bases to about 90 bases, about 90 bases to about 100 bases, about 30, 35, 40, 45, 50, 60, 70, 80, 90, or 100 bases.

In any of the foregoing embodiments, each of the plurality of second indexing primers can have a length of from about 20 bases to about 100 bases. By way of example, but not limitation, each of the plurality of second indexing primers can have a length from about 20 bases to about 100 bases, about 20 bases to about 90 bases, about 20 bases to about 80 bases, about 20 bases to about 70 bases, about 20 bases to about 60 bases, about 20 bases to about 50 bases, about 20 bases to about 40 bases, about 20 bases to about 30 bases, about 25 bases to about 100 bases, about 25 bases to about 90 bases, about 25 bases to about 80 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 70 bases, about 50 bases to about 60 bases, about 60 bases to about 100 bases, about 60 bases to about 90 bases, about 60 bases to about 80 bases, about 60 bases to about 70 bases, about 70 bases to about 100 bases, about 70 bases to about 90 bases, about 70 bases to about 80 bases, about 80 bases to about 100 bases, about 80 bases to about 90 bases, about 90 bases to about 100 bases, about 30, 35, 40, 45, 50, 60, 70, 80, 90, or 100 bases.

In any of the foregoing embodiments, each of the plurality of first indexing primers can further include the sequence of SEQ ID NO: 87. In any of the foregoing embodiments, each of the plurality of second indexing primers can further include the sequence of SEQ ID NO: 78.

In any of the foregoing embodiments, the first common nucleotide sequence and the first 3′ terminal portion can have a melting temperature (Tₘ) greater than the ligation temperature. By way of example, but not limitation, the melting temperature of the first common nucleotide sequence and the first 3′ terminal portion can be 1° C., 2° C., 3° C., 4° C., 5° C., 6° C., 7° C., 8° C., 9° C., 10° C., 15° C., 20° C., 25° C., 30° C., 35° C., or 40° C. higher than the ligation temperature. By way of further example, but not limitation, the first common nucleotide sequence and the first 3′ terminal portion can have a Tₘ of greater than 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56 ° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C., or of about 40° C. to about 80° C., about 40° C. to about 70° C., about 40° C. to about 60° C., about 40° C. to about 50° C., about 50° C. to about 80° C., about 50° C. to about 70° C., about 50° C. to about 60° C., about 60° C. to about 80° C., about 60° C. to about 70° C., or about 70° C. to about 80° C. It should be understood that the first common nucleotide sequence the first 3′ terminal portion should have a Tₘ below the first denaturation temperature, the second denaturation temperature, and the third denaturation temperature.

In any of the foregoing embodiments, the ligation temperature can be any temperature sufficient for the ligase to ligate one of the plurality of first indexing primers to the first 5′ end of the first strand or the second 5′ end of second strand. It should be further understood that the ligation temperature cannot be higher than the Tₘ of the partially double-stranded DNA substrate. If the ligation temperature is higher than the Tₘ of the partially double-stranded DNA substrate, it is possible then for the plurality of first indexing primers to act as primers rather as a ligated 5′ adapter. By way of example, but not limitation, the ligation temperature can be about 25° C. to about 40° C. By way of further example, but not limitation, the ligation temperature can be about 25° C. to about 40° C., about 25° C. to about 37° C., about 25° C. to about 35° C., about 25° C. to about 30° C., about 30° C. to about 40° C., about 30° C. to about 35° C., about 35° C. to about 40° C., about 25° C., 26° C., 27° C., 28° C., 29° C., 30° C., 31° C., 32° C., 33° C., 34° C., 35° C., 36° C., 37° C., 38° C., 39° C., or 40° C.

In any of the foregoing embodiments, the ligation duration and be any time sufficient to ligate one of the plurality of first indexing primers to the first 5′ end of the first strand or the second 5′ end of the second strand. In any of the foregoing embodiments, the ligation duration can be from about 5 minutes to about 60 minutes. By way of example, but not limitation, the ligation duration can be about 5 minutes to about 60 minutes, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 60 minutes, about 10 minutes to about 50 minutes, about 10 minutes to about 40 minutes, about 10 minutes to about 30 minutes, about 10 minutes to about 20 minutes, about 20 minutes to about 60 minutes, about 20 minutes to about 50 minutes, about 20 minutes to about 40 minutes, about 20 minutes to about 30 minutes, about 30 minutes to about 60 minutes, about 30 minutes to about 50 minutes, about 30 minutes to about 40 minutes, about 40 minutes to about 60 minutes, about 40 minutes to about 50 minutes, about 50 minutes to about 60 minutes, about 5, 10, 15, 20, 25, 30, 40, 50, or 60 minutes.

In any of the foregoing embodiments, any suitable ligase can be used. It should be understood that the ligase should can be a ligase that is a thermolabile ligase capable of ligation in low magnesium buffer and be inactivated at the first denaturation temperature for the first denaturation duration. It should be understood that such low magnesium buffer conditions are those suitable for PCR. By way of example, but not limitation, the ligase can be T3 DNA ligase. In any of the foregoing embodiments, by way of example, but not limitation, the ligase can be added at about 30 to about 300 enzyme units per μL of the first reaction mixture.

In any of the foregoing embodiments, any suitable polymerase can be used. In some embodiments, the polymerase is not active at the ligation temperature. By way of example, but not limitation, the polymerase can further include a hot start antibody or aptamer. In any of the foregoing embodiments, the polymerase can be a hot start polymerase with an activation temperature, where the activation temperature is less than the first denaturation temperature. By way of example, but not limitation, the activation temperature can be less than the first denaturation temperature, second denaturation temperature, and third denaturation temperature. By way of example but not limitation, the polymerase can be a thermostable DNA polymerase with 3′-5′ exonuclease proofreading activity selected from the group consisting of Kapa HiFi DNA Polymerase (Roche), NEB Q5 DNA Polymerase (NEB), PrimeStar GXL DNA Polymerase (Takara), or High Fidelity DNA Polymerase (Qiagen). In any of the foregoing embodiments, the DNA polymerase can further include a hot start antibody or aptamer that increases the activation temperature of the DNA polymerase. In such embodiments, the DNA polymerase can be Kapa HiFi Hot Start DNA Polymerase (Roche), NEB Q5 Hot Start DNA Polymerase (NEB), PrimeStar GXL Hot Start DNA Polymerase (Takara), or High Fidelity Hot Start DNA Polymerase (Qiagen).

In any of the foregoing embodiments, the first denaturation duration, the second denaturation duration, and the third denaturation duration can each be from about 30 seconds to about 2 minutes. By way of example, but not limitation, the first denaturation duration, the second denaturation duration, and the third denaturation duration can each be from about 30 seconds to about 2 minutes, about 30 seconds to 1.5 minutes, about 30 seconds to 1 minute, about 1 minute to about 2 minutes, about 1 minutes to about 1.5 minutes, about 1.5 minutes to about 2 minutes, about 30 seconds, 45 seconds, 1 minutes, 1.5 minutes, or 2 minutes.

In any of the foregoing embodiments, the first denaturation temperature, the second denaturation temperature and the third denaturation temperature can each be from about 95° C. to about 98° C. It should be understood that any suitable temperature for denaturing double-stranded DNA to be annealed to in each PCR cycle can be used. It should be understood that the first denaturation temperature should be sufficient that it is higher than a melting temperature of the first strand and second strand. One of skill in the art can readily determine such temperature and the necessary time through routine experimentation and/or knowledge in the art.

In any of the foregoing embodiments, the first annealing temperature, the second annealing temperature, and the third annealing temperature can each be from about 55° C. to about 65° C. By way of example, but not limitation, the first annealing temperature, the second annealing temperature, and the third annealing temperature can each be from about 55° C. to about 65° C., 55° C. to about 60° C. , about 60° C. to about 65° C., about 55° C., 56° C., 57° C., 58° C., 59° C., 60° C., 61° C., 62° C., 63° C., 64° C., or 65° C.

In any of the foregoing embodiments, the first annealing duration, the second annealing duration, and the third annealing duration can each be from about 10 seconds to about 60 seconds. It should be understood that the first annealing temperature, the second annealing temperature, and the third annealing temperature and the first annealing duration, the second annealing duration, and the third annealing duration should be sufficient for the annealing required in each respective PCR step to occur. One of skill in the art can readily determine such temperature and the necessary time through routine experimentation and/or knowledge in the art.

In any of the foregoing embodiments, the first extension temperature, the second extension temperature, and the third extension temperature can each be from about 60° C. to about 72° C. By way of example, but not limitation, the first extension temperature, the second extension temperature, and the third extension temperature can be about 60° C., 61° C. 62° C., 63° C., 64° C., 65° C., 66° C., 67° C., 68° C., 69° C., 70° C., 71° C., or 72° C.

In any of the foregoing embodiments, the first extension duration, the second extension duration, and the third extension duration can each be from about 30 seconds to about 5 minutes. It should be understood that the first extension temperature, the second extension temperature, and the third extension temperature and the first extension duration, the second extension duration, and the third extension duration should be sufficient for the extension required in each respective PCR step to occur. One of skill in the art can readily determine such temperature and the necessary time through routine experimentation and/or knowledge in the art.

In any of the foregoing embodiments, the plurality of first indexing primers and the plurality of second indexing primers can be added to the first reaction mixture at about 100 nM to about 1 μM. By way of example, but not limitation, the plurality of first indexing primers and the plurality of second indexing primers can be added to the first reaction mixture at about 100 nM to about 200 nM.

In any of the foregoing embodiments, the partially double-stranded DNA substrate can be derived from genomic DNA, cDNA from reverse transcription of RNA, whole genome amplification (WGA), multiplex PCR, or synthetic DNA.

Methods Using a Blocker Oligonucleotide

In any of the foregoing embodiments, step (ii) can further include adding a blocker oligonucleotide to the first reaction mixture, where the blocker oligonucleotide includes a 5′ portion that is at least partially complementary to at least a portion of the second 3′ terminal portion of each of the plurality of second indexing primers, where a melting temperature of the blocker oligonucleotide and each of the plurality of second indexing primers is greater than the ligation temperature and less than the first annealing temperature and the third annealing temperature. In such embodiments, the blocker oligonucleotide can be added in an amount sufficient to inhibit ligation of each of the plurality of second indexing primers to the first 5′ end of the first strand and the second 5′ end of the second strand. In some embodiments, the blocker oligonucleotide include a 5′ portion that is fully complementary to at least a portion of the second 3′ terminal portion of each of the plurality of second indexing primers.

In any of the foregoing embodiments, the melting temperature of the blocker oligonucleotide and each of the plurality of second indexing primers can be greater than a melting temperature of the blocker oligonucleotide and each of the plurality of first indexing primers.

In any of the foregoing embodiments, the blocker oligonucleotide can be added in an amount that is 1×, 1.5×, 2×, 4× or 6× an amount of the plurality of second indexing primers added in step (ii). By way of example, but not limitation, the blocker oligonucleotide can be added in an amount that is 1× to about 6 ×, about 1.5 × to about 6×, about 2× to about 6×, about 4× to about 6×, 1× to about 4×, about 1.5× to about 4×, about 2× to about 4×, 1× to about 2×, 1× to about 1.5×, or about 1.5 × to about 2× the amount of the plurality of second indexing primers added in step (ii). Alternatively, the plurality of second indexing primers can be pre-annealed to the blocker oligonucleotide prior to adding the plurality of second indexing primers in step (ii).

In any of the foregoing embodiments, the blocker oligonucleotide can further include a first additional portion positioned 3′ to the 5′ portion which is complementary to each of the plurality of second indexing primers and not complementary to each of the plurality of first indexing primers. In any of the foregoing embodiments, the blocker oligonucleotide can include from about 14 bases to about 200 bases. By way of example, but not limitation, the blocker oligonucleotide can have a length from about 14 bases to about 200 bases, about 14 bases to about 150 bases, about 14 bases to about 100 bases, about 14 bases to about 50 bases, about 14 bases to about 25 bases, about 25 bases to about 200 bases, about 25 bases to about 150 bases, about 25 bases to about 100 bases, about 25 bases to about 50 bases, about 50 bases to about 200 bases, about 50 bases to about 150 bases, about 50 bases to about 100 bases, about 100 bases to about 200 bases, about 100 bases to about 150 bases, about 150 bases to about 200 bases, about 14, 20, 25, 30, 40, 50, 60, 70, 80, 90, 100, 150, or 200 bases. It should be understood that these lengths are exemplary and that the blocker oligonucleotide can be any suitable length.

In any of the foregoing embodiments a melting temperature between the first additional portion and each of the plurality of second indexing primers can be about the same or greater than a melting temperature between the 5′ portion and each of the plurality of first indexing primers and each of the plurality of second indexing primers. By way of example, but not limitation, the melting temperature between the first additional portion and each of the plurality of second indexing primers can be at least 1° C. greater than a melting temperature between the 5′ portion and each of the plurality of first indexing primers and each of the plurality of second indexing primers. By way of example, but not limitation, the melting temperature between the first additional portion and each of the plurality of second indexing primers can be at least 1° C., 2° C., 3° C., 4° C., 5° C., 10° C., 15° C. or 20° C. greater than a melting temperature between the 5′ portion and each of the plurality of first indexing primers and each of the plurality of second indexing primers

In any of the foregoing embodiments, the blocker oligonucleotide can include a second additional portion positioned between the 5′ portion and the first additional portion, where the second additional portion is not complementary to each of the plurality of first indexing primers and is not complementary to each of the plurality of second primers. By way of example, but not limitation, the second additional portion can have a length from about 1 base to about 30 bases. By way of further example, but not limitation, the second additional portion can have a length from about 1 base to about 30 bases, about 1 base to about 25 bases, about 1 base to about 20 bases, about 1 base to about 15 bases, about 1 base to about 10 bases, about 1 base to about 5 bases, about 5 bases to about 30 bases, about 5 bases to about 25 bases, about 5 bases to about 20 bases, about 5 bases to about 15 bases, about 5 bases to about 10 bases, about 10 bases to about 30 bases, about 10 bases to about 25 bases, about 10 bases to about 20 bases, about 10 bases to about 15 bases, about 15 bases to about 30 bases, about 15 bases to about 25 bases, about 15 bases to about 20 bases, about 20 bases to about 30 bases, about 20 bases to about 25 bases, about 25 bases to about 30 bases, about 1, 2, 3, 4, 5, 6, 7, 8, 9, 10, 15, 20, 25, or 30 bases.

In any of the foregoing embodiments, the blocker oligonucleotide can comprise mismatches in its sequence that render it only partially complementary to each of the plurality of second indexing primers. This can be used to reduce the melting temperature of the blocker oligonucleotide and each of the plurality of second indexing primers so that the blocker is not active during PCR steps, e.g. steps (iv)-(iv).

In any of the foregoing embodiments, the blocker oligonucleotide can include a sequence that is not complementary to each of the plurality of second indexing primers. The sequence that is not complementary to each of the plurality of second indexing primers can have a length from about 1 to about 30 bases. By way of example, but not limitation, the sequence that is not complementary to each of the plurality of second indexing primers can have a length from about 1 base to about 30 bases. By way of further example, but not limitation, the second additional portion can have a length from about 1 base to about 30 bases, about 1 base to about 25 bases, about 1 base to about 20 bases, about 1 base to about 15 bases, about 1 base to about 10 bases, about 1 base to about 5 bases, about 5 bases to about 30 bases, about 5 bases to about 25 bases, about 5 bases to about 20 bases, about 5 bases to about 15 bases, about 5 bases to about 10 bases, about 10 bases to about 30 bases, about 10 bases to about 25 bases, about 10 bases to about 20 bases, about 10 bases to about 15 bases, about 15 bases to about 30 bases, about 15 bases to about 25 bases, about 15 bases to about 20 bases, about 20 bases to about 30 bases, about 20 bases to about 25 bases, about 25 bases to about 30 bases, about 1, 2, 3, 4, 5, 6, 7, 8, 9, 10, 15, 20, 25, or 30 bases.

In any of the foregoing embodiments, the blocker oligonucleotide can further include a 3′ modification to block polymerase extension if it does not include a hairpin portion. By way of example, but not limitation, the 3′ modification can be a C3 carbon spacer, hexanediol, spacer 9, spacer 18, phosphate, 2′,3′-dideoxynucleosides ddA, ddT, ddC and ddG, 3′-deoxynucleosides 3′-A, 3′-T, 3′-C and 3′-G, RNA nucleotides such as rU, 3-O-methyl nucleotides, or a DNA sequence that is not complementary to the adjacent primer sequence such as poly T, poly A, poly C and poly G and additionally comprises nuclease resistant linkages to prevent proofreading polymerase 3′-5′ exonuclease activity from removing the DNA sequence that is not complementary to the adjacent primer sequence.

In any of the foregoing embodiments, a melting temperature between the blocker oligonucleotide and each of the plurality of first indexing primers is less than a melting temperature between the blocker oligonucleotide and each of the plurality of second indexing primers, By way of example, but not limitation, the melting temperature between the blocker oligonucleotide and each of the plurality of first indexing primers can be at least 5° C. less than a melting temperature between the blocker oligonucleotide and each of the plurality of second indexing primers. By way of further example but not limitation, the melting temperature between the blocker oligonucleotide and each of the plurality of first indexing primers can be at least 5° C., 6° C., 7° C., 8° C. , 9° C., 10° C., 15° C., 20° C. or more less than a melting temperature between the blocker oligonucleotide and each of the plurality of second indexing primers.

In any of the foregoing embodiments, the 5′ portion of the blocker oligonucleotide can include the sequence of SEQ ID NO: 127. In such instances, each of the plurality of second indexing primers can include the sequence of SEQ ID NO: 126.

In any of the foregoing embodiments, the blocker oligonucleotide can further include a hairpin portion positioned 3′ to the first additional portion where the hairpin portion includes a first hairpin sequence and a second hairpin sequence, the first hairpin sequence being positioned 5′ to the second hairpin sequence, where the first hairpin sequence and the second hairpin sequence are complementary, and where the hairpin portion has a melting temperature greater than the first annealing temperature, the second annealing temperature, and the third annealing temperature. In such embodiments, the blocker oligonucleotide further include a 3′ hydroxyl group.

In any of the foregoing embodiments, the hairpin portion can further include a third hairpin sequence between the first hairpin sequence and the second hairpin sequence. In such embodiments, the third sequence can form a loop sufficient to allow formation of a stable stem-loop structure by the hairpin portion and the first additional portion. In such embodiments, the third hairpin sequence can have a length from about 4 to about 20 bases or more. It should be understood that the third hairpin sequence can form a loop sufficient to allow formation of a stable stem-loop structure with the first and second hairpin sequences.

In any of the foregoing embodiments, a melting temperature of the hairpin portion can be greater than a melting temperature between the 5′ portion and each of the plurality of second indexing primers, the additional portion and each of the plurality of second indexing primers or both.

In any of the foregoing embodiments, the second set of conditions can be further sufficient for the DNA polymerase to extend a 3′ end of the blocker (hairpin) oligonucleotide to yield an extended hairpin blocker. In such embodiments, the extended hairpin blocker can have a stabilized secondary structure that provides it with a higher melting temperature than the second annealing temperature and the third annealing temperature. By way of example, but not limitation, the melting temperature of the extended hairpin blocker can be at least 1° C., 2° C., 3° C., 4° C., 5° C., 6° C., 7° C., 8° C., 9° C., 10° C., 15° C., 20° C., 25° C., or 30° C. higher than second annealing temperature and the third annealing temperature.

In any of the foregoing embodiments, the blocker oligonucleotide can further include dU bases, where step (ii) further includes adding uracil DNA glycosylase to the first reaction mixture, the first set of conditions in step (iii) is further sufficient for the UDG to excise the dU bases and create abasic site in the blocker oligonucleotide, and wherein in step (iv) the second set of conditions are further sufficient to inactivate the UDG enzyme.

In any of the foregoing embodiments, the method can further include sequencing the fifth strand and seventh strand or the sixth strand and eighth strand.

In any of the foregoing embodiments, the first common nucleotide sequence and the first 3′ terminal portion can have a melting temperature greater than the ligation temperature but where the melting temperature is below the first annealing temperature. In such embodiments, the melting temperature of the first common nucleotide sequence and the first 3′ terminal portion can be at least 1° C. above the ligation temperature. By way of example, but not limitation, the melting temperature of the first common nucleotide sequence and the first 3′ terminal portion can be at least 1° C., 2° C., 3° C., 4° C., 5° C., 10° C., 15° C. or 20° C. greater than the ligation temperature. By way of further example, but not limitation, the melting temperature of the first common nucleotide sequence and the first 3′ terminal portion can be at least 1° C. below the first annealing temperature and optionally, the second annealing temperature and third annealing temperature. By way of further example, but not limitation the melting temperature of the first common nucleotide sequence and the first 3′ terminal portion can be at least 1° C., 2° C., 3° C., 4° C., 5° C., 10° C., 15° C. or 20° C. less than the first annealing temperature, and optionally the second annealing temperature or the third annealing temperature.

In some embodiments, the first 3′ terminal portion and the second 3′ terminal portion can have melting temperature lower than the first annealing temperature. In such embodiments, the second portion and fourth portion can further include the second common nucleotide sequence and each of the plurality of second indexing primers can be complementary to the second nucleotide sequence with a melting temperature between the second indexing primer and the first common nucleotide sequence and the second common nucleotide sequence being higher than the first annealing temperature.

In any of the foregoing embodiments, the blocker oligonucleotide and each of the plurality of second indexing primers can have a lower melting temperature than a melting temperature of each of the plurality of second indexing primers and the partially double-stranded DNA substrate.

Methods for Ligation-Coupled PCR—Both Indexing Primers Do Not Include a Common 3′ Terminal Sequence; Hairpin Adapter is Used

In some embodiments, a method for ligation-coupled PCR can include providing a partially double-stranded DNA substrate that includes a first strand and a second strand, the partially double-stranded DNA substrate including a first 3′ overhang, a double-strand portion, and a second 3′ overhang, the first strand comprising, in a 5′ to 3′ direction, a first 5′ end, a first portion, and a second portion, the second strand comprising, in a 5′ to 3′ direction, a second 5′ end, a third portion, and a fourth portion, where the first portion of the first strand and the third portion of the second strand are complementary and form the double-stranded portion, where the second portion of the first strand forms the first 3′ overhang, where the fourth portion of the second strand forms the second 3′ overhang, where the second portion of the first strand and the fourth portion of the second strand each comprise a first common nucleotide sequence positioned at a 5′ end of the first 3′ overhang and the second 3′ overhang, respectively, and where the second portion of the first strand and the fourth portion of the second strand each include a second common nucleotide sequence positioned 3′ to the first common nucleotide sequence; adding a plurality of first indexing primers, a plurality of second indexing primers, a plurality of 5′ adapters, a ligase, a DNA polymerase and deoxynucleotide triphosphates (dNTPs) to the partially double-stranded DNA substrate to yield a first reaction mixture, where each of the plurality of first indexing primers comprise a first 3′ terminal portion that is not complementary to the first common nucleotide sequence, wherein each of the plurality of second indexing primers comprises a second 3′ terminal portion that is not complementary to the first common nucleotide sequence, wherein the second 3′ terminal sequence is complementary to at the second common nucleotide sequence, and wherein each of the plurality of 5′ adapters comprises a third 3′ terminal sequence complementary to the first common nucleotide sequence, a first 5′ portion positioned 5′ to the third 3′ terminal sequence which comprises at least a portion of the first 3′ terminal portion of each of the plurality of first indexing primers, a second 5′ portion positioned 5′ to the first 5′ portion and complementary to the first 5′ portion, and a replication blocker capable of blocking the DNA polymerase positioned at a 5′ end of the first 5′ portion, and wherein the 5′ adapter can form a hairpin formed by annealing of the first 5′ portion to the second 5′ portion; incubating the first reaction mixture under a first set of conditions comprising a ligation temperature for a ligation duration, sufficient: 1) for the third 3′ terminal portion to anneal to the first common nucleotide sequence, and 2) for the ligase to ligate one of the plurality of 5′ adapters to the first 5′ end of the first strand and the second 5′ end of the second strand, to yield a second reaction mixture comprising: a third strand comprising, in a 5′ to 3′ direction, one of the plurality of 5′ adapters, the first portion, and the second portion, and a fourth strand comprising, in a 5′ to 3′ direction, one of the plurality of 5′ adapters, the third portion, and the fourth portion; incubating the second reaction mixture under a second set of conditions comprising a first denaturation temperature for a first denaturation duration, a first annealing temperature for a first annealing duration, and a first extension temperature for a first extension duration, sufficient: a) to inactivate the ligase, denature double-stranded DNA and, optionally, to activate the DNA polymerase, b) for the second 3′ terminal portion of one of the plurality of second indexing primers to anneal to the second common nucleotide sequence of the second portion of the third strand and for one of the plurality of second indexing primers to anneal to the second common nucleotide sequence of the fourth portion of the fourth strand, and c) for the DNA polymerase to extend the one of the plurality of second indexing primers annealed to the second common nucleotide sequence of the second portion of the third strand and to extend the one of the plurality of second indexing primers annealed to the second common nucleotide sequence of the fourth portion of the fourth strand, to yield a third reaction mixture comprising the third strand, the fourth strand, a fifth strand, and a sixth strand, the fifth strand comprising, in a 5′ to 3′ direction, one of the plurality of second indexing primers, the third portion, a reverse complement of the first common nucleotide sequence, and a reverse complement of the first 5′ portion of one of the plurality of 5′ adapters, and the sixth strand comprising, in a 5′ to 3′ direction, one of the plurality of second indexing primers, the first portion, the reverse complement of the first common nucleotide sequence, and the reverse complement of the first 5′ portion of one of the plurality of 5′ adapters; incubating the third reaction mixture under a third set of conditions comprising a second denaturation temperature for a second denaturation duration, a second annealing temperature for a second annealing duration, a second extension temperature for a second extension duration, sufficient: a) to denature any double-stranded DNA, b) for the first 3′ terminal portion of one of the plurality of first indexing primers to anneal to the reverse complement of the first 5′ portion of one of the plurality of 5′ adapters of the fifth strand and one of the plurality of first indexing primers to anneal to the reverse complement of the first 5′ portion of one of the plurality of 5′ adapters of the sixth strand, and c) for the DNA polymerase to extend the one of the plurality of first indexing primers annealed to the reverse complement of the first 5′ portion of one of the plurality of 5′ adapters of the fifth strand and to extend the one of the plurality of first indexing primers annealed to the reverse complement of the first 5′ portion of one of the plurality of 5′ adapters of the sixth strand, to yield a fourth reaction mixture comprising the fifth strand, the sixth strand, a seventh strand and an eighth strand, the seventh strand comprising, in a 5′ to 3′ direction, one of the plurality of first indexing primers, the first common nucleotide sequence, the first portion, and and a reverse complement of one of the plurality of second indexing primers, and the eighth strand comprising, in a 5′ to 3′ direction, one of the plurality of first indexing primers, the first common nucleotide sequence, the third portion, the reverse complement of one of the plurality of second indexing primers; incubating the fourth reaction mixture under a fourth set of conditions comprising a third denaturation temperature for a third denaturation duration, a third annealing temperature for a third annealing duration, and a third extension temperature for a third extension duration, sufficient: a) to denature any double-stranded DNA, b) for one of the plurality of second indexing primers to anneal to the reverse complement of one of the plurality of second indexing primers of the seventh strand and for one of the plurality of second indexing primers to anneal to the reverse complement of one of the plurality of second indexing primers of the eighth strand, and c) for the DNA polymerase to extend the one of the plurality of second indexing primers annealed to each of the seventh strand and eighth strand to yield a fifth reaction mixture comprising the seventh strand, the eighth strand, a ninth strand and a tenth strand, the ninth strand comprising, in a 5′ to 3′ direction, one of the plurality of second indexing primers, the third portion, the reverse complement of the first common nucleotide sequence, and a reverse complement of one of the plurality of first indexing primers, and the tenth strand comprising, in a 5′ to 3′ direction, one of the plurality of second indexing primers, the first portion, the reverse complement of the first common sequence, and the reverse complement of one of the plurality of first indexing primers, wherein the seventh strand and ninth strand are complementary and wherein the eighth strand and tenth strand are complementary; and incubating the fifth reaction mixture under a fifth set of conditions comprising a fourth denaturation temperature for a fourth denaturation duration, a fourth annealing temperature for a fourth annealing duration, and a fourth extension temperature for a fourth extension duration, sufficient for at least a portion of the plurality of first indexing primers, at least a portion of the plurality of second indexing primers and the DNA polymerase to amplify the seventh strand and ninth strand and eighth strand and tenth strand.

In the foregoing embodiment, it should be understood that the second common nucleotide sequence can be separate from the first common nucleotide sequence or can overlap or be a 3′ portion of the first common nucleotide sequence. Thus, in some embodiments, each of the plurality of second indexing primers can anneal to a 3′ terminal portion of the first common nucleotide sequence such that the one of the plurality of second indexing primers cannot ligate to the first strand or the second strand, but can still act as a primer. Alternatively, in some embodiments the second common nucleotide sequence can be separate from the first common nucleotide sequence.

In any of the foregoing embodiments, steps (i)-(vii) can be performed in a single closed tube. In any of the foregoing embodiments, the method can not include any purification steps between steps (i)-(vii).

In any of the foregoing embodiments, where a plurality of 5′ adapter is used for 5′ adapter ligation, the first 5′ portion and the second 5′ portion can each have a length of about 12 bases to about 20 bases.

In any of the foregoing embodiments, where a plurality of 5′ adapter is used for 5′ adapter ligation, each of the plurality of 5′ adapters can further include an intervening sequence between the first 5′ portion and the second 5′ portion. By way of example, but not limitation, the intervening sequence can have a length from about 4 bases to about 20 bases.

In any of the foregoing embodiments, where a plurality of 5′ adapter is used for 5′ adapter ligation, the replication block can be selected from a stable abasic site, a C3 spacer, hexandiol, Spacer 9, Spacer 18, 3 or more rU bases, and 2′-O-methyl RNA bases.

In any of the foregoing embodiments, where a plurality of 5′ adapter is used for 5′ adapter ligation, each of the plurality of 5′ adapters can have a length of about 25 bases to about 100 bases. By way of example, but not limitation, each of the plurality of 5′ adapters can have a length of about 25 bases to about 100 bases, about 30 bases to about 100 bases, about 40 bases to about 100 bases, about 50 bases to about 100 bases, about 60 bases to about 100 bases, about 70 bases to about 100 bases, about 80 bases to about 100 bases, about 90 bases to about 100 bases, about 25, 30, 40, 50, 60, 70, 80, 90 or 100 bases.

In any of the foregoing embodiments, where a plurality of 5′ adapter is used for 5′ adapter ligation, the partially double-stranded DNA substrate can have a length of about 24 bases to about 6000 bases. By way of example, but not limitation, the partially double-stranded DNA substrate can have a length of about 24 bases to about 6000 bases, about 24 bases to about 5500 bases, about 24 bases to about 5000 bases, about 24 bases to about 4500 bases, about 24 bases to about 4000 bases, about 24 bases to about 3500 bases, about 24 bases to about 3000 bases, about 24 bases to about 2500 bases, about 24 bases to about 2000 bases, about 24 bases to about 1500 bases, about 24 bases to about 1000 bases, about 24 bases to about 750 bases, about 24 bases to about 500 bases, about 24 bases to about 250 bases, about 24 bases to about 200 bases, about 24 bases to about 100 bases, about 24 bases to about 50 bases, about 100 to about 6000 bases, about 100 to about 5500 bases, about 100 to about 5000 bases, about 100 to about 4500 bases, about 100 to about 4000 bases, about 100 to about 3500 bases, about 100 to about 3000 bases, about 100 to about 2500 bases, about 100 to about 2000 bases, about 100 to about 1500 bases, about 100 to about 1000 bases, about 100 to about 750 bases, about 100 to about 500 bases, about 100 to about 250 bases, about 100 to about 200 bases, about 200 bases to about 6000 bases, about 200 baes to about 5500 bases, about 200 bases to about 5000 bases, about 200 to about 4500 bases, about 200 to about 4000 bases, about 200 to about 3500 bases, about 200 to about 3000 bases, about 200 to about 2500 bases, about 200 to about 2000 bases, about 200 to about 1500 bases, about 200 to about 1000 bases, about 200 to about 750 bases, about 200 to about 500 bases, about 200 to about 250 bases, about 250 bases to about 6000 bases, about 250 to about 5500 bases, about 250 to about 5000 bases, about 250 to about 4500 bases, about 250 to about 4000 bases, about 250 to about 3500 bases, about 250 to about 3000 bases, about 250 to about 2500 bases, about 250 bases to about 2000 bases, about 250 to about 1500 bases, about 250 to about 1000 bases, about 250 to about 750 bases, about 250 to about 500 bases, about 500 bases to about 6000 bases, about 500 bases to about 5500 bases, about 500 bases to about 5000 bases, about 500 bases to about 4500 bases, about 500 bases to about 4000 bases, about 500 bases to about 3500 bases, about 500 bases to about 3000 bases, about 500 bases to about 2500 bases, about 500 bases to about 2000 bases, about 500 bases to about 1500 bases, about 500 bases to about 1000 bases, about 500 bases to about 750 bases, about 750 bases to about 6000 bases, about 750 bases to about 5500 bases, about 750 bases to about 5000 bases, about 750 bases to about 4500 bases, about 750 bases to about 4000 bases, about 750 bases to about 3500 bases, about 750 bases to about 3000 bases, about 750 bases to about 2500 bases, about 750 bases to about 2000 bases, about 750 bases to about 1500 bases, about 750 bases to about 1000 bases, about 1000 bases to about 6000 bases, about 1000 bases to about 5500 bases, about 1000 bases to about 5000 bases, about 1000 bases to about 4500 bases, about 1000 bases to about 4000 bases, about 1000 bases to about 3500 bases, about 1000 bases to about 3000 bases, about 1000 bases to about 2500 bases, about 1000 bases to about 2000 bases, about 1000 bases to about 1500 bases, about 1500 bases to about 6000 bases, about 1500 bases to about 5500 bases, about 1500 bases to about 5000 bases, about 1500 bases to about 4500 bases, about 1500 bases to about 4000 bases, about 1500 bases to about 3500 bases, about 1500 bases to about 3000 bases, about 1500 bases to about 2500 bases, about 1500 bases to about 2000 bases, about 2000 bases to about 6000 bases, about 2000 bases to about 5500 bases, about 2000 bases to about 5000 bases, about 2000 bases to about 4500 bases, about 2000 bases to about 4000 bases, about 2000 bases to about 3500 bases, about 2000 bases to about 3000 bases, about 2000 bases to about 2500 bases, about 2500 bases to about 6000 bases, about 2500 bases to about 5500 bases, about 2500 bases to about 5000 bases, about 2500 bases to about 4500 bases, about 2500 bases to about 4000 bases, about 2500 bases to about 3500 bases, about 2500 bases to about 3000 bases, about 3000 bases to about 6000 bases, about 3000 bases to about 5500 bases, about 3000 bases to about 5000 bases, about 3000 bases to about 4500 bases, about 3000 bases to about 4000 bases, about 3000 bases to about 3500 bases, about 3500 bases to about 6000 bases, about 3500 bases to about 5500 bases, about 3500 bases to about 5000 bases, about 3500 to about 4500 bases, about 3500 to about 4000 bases, about 4000 bases to about 6000 bases, about 4000 bases to about 5500 bases, about 4000 bases to about 5000 bases, about 4000 bases to about 4500 bases, about 4500 bases to about 6000 bases, about 4500 bases to about 5500 bases, about 4500 bases to about 5000 bases, about 5000 bases to about 6000 bases, about 5000 bases to about 5500 bases, about 5500 to about 6000 bases, about 24, 30, 40, 50, 60, 70, 80, 90, 100, 200, 250, 500, 750, 1000, 1500, 2000, 2500, 3000, 3500, 4000, 4500, 5000, 5500, or 6000 bases. It should be understood that the length of the partially double-stranded DNA substrate are exemplary and that other sizes are within the scope of the present disclosure. It should be understood that the length of the partially double-stranded DNA substrate refers to the length of the first strand or the second strand of the partially double-stranded DNA substrate, i.e. from the first 5′ end to the 3′ end of the first 3′ overhang or from the second 5′ end to the 3′ end of the second 3′ overhang.

In any of the foregoing embodiments, where a plurality of 5′ adapter is used for 5′ adapter ligation, the first portion and the third portion of the first strand and second strand, respectively, can have a length of about 20 bases to about 6000 bases. By way of example, but not limitation, the first portion of the first oligonucleotide and the third portion of the second oligonucleotide can each have a length of about 20 bases to about 6000 bases, about 20 bases to about 5500 bases, about 20 bases to about 5000 bases, about 20 bases to about 4500 bases, about 20 bases to about 4000 bases, about 20 bases to about 3500 bases, about 20 bases to about 3000 bases, about 20 bases to about 2500 bases, about 20 bases to about 2000 bases, about 20 bases to about 1500 bases, about 20 bases to about 1000 bases, about 20 bases to about 750 bases, about 20 bases to about 500 bases, about 20 bases to about 250 bases, about 20 bases to about 200 bases, about 20 bases to about 100 bases, about 20 bases to about 50 bases, about 100 to about 6000 bases, about 100 to about 5500 bases, about 100 to about 5000 bases, about 100 to about 4500 bases, about 100 to about 4000 bases, about 100 to about 3500 bases, about 100 to about 3000 bases, about 100 to about 2500 bases, about 100 to about 2000 bases, about 100 to about 1500 bases, about 100 to about 1000 bases, about 100 to about 750 bases, about 100 to about 500 bases, about 100 to about 250 bases, about 100 to about 200 bases, about 200 bases to about 6000 bases, about 200 baes to about 5500 bases, about 200 bases to about 5000 bases, about 200 to about 4500 bases, about 200 to about 4000 bases, about 200 to about 3500 bases, about 200 to about 3000 bases, about 200 to about 2500 bases, about 200 to about 2000 bases, about 200 to about 1500 bases, about 200 to about 1000 bases, about 200 to about 750 bases, about 200 to about 500 bases, about 200 to about 250 bases, about 250 bases to about 6000 bases, about 250 to about 5500 bases, about 250 to about 5000 bases, about 250 to about 4500 bases, about 250 to about 4000 bases, about 250 to about 3500 bases, about 250 to about 3000 bases, about 250 to about 2500 bases, about 250 bases to about 2000 bases, about 250 to about 1500 bases, about 250 to about 1000 bases, about 250 to about 750 bases, about 250 to about 500 bases, about 500 bases to about 6000 bases, about 500 bases to about 5500 bases, about 500 bases to about 5000 bases, about 500 bases to about 4500 bases, about 500 bases to about 4000 bases, about 500 bases to about 3500 bases, about 500 bases to about 3000 bases, about 500 bases to about 2500 bases, about 500 bases to about 2000 bases, about 500 bases to about 1500 bases, about 500 bases to about 1000 bases, about 500 bases to about 750 bases, about 750 bases to about 6000 bases, about 750 bases to about 5500 bases, about 750 bases to about 5000 bases, about 750 bases to about 4500 bases, about 750 bases to about 4000 bases, about 750 bases to about 3500 bases, about 750 bases to about 3000 bases, about 750 bases to about 2500 bases, about 750 bases to about 2000 bases, about 750 bases to about 1500 bases, about 750 bases to about 1000 bases, about 1000 bases to about 6000 bases, about 1000 bases to about 5500 bases, about 1000 bases to about 5000 bases, about 1000 bases to about 4500 bases, about 1000 bases to about 4000 bases, about 1000 bases to about 3500 bases, about 1000 bases to about 3000 bases, about 1000 bases to about 2500 bases, about 1000 bases to about 2000 bases, about 1000 bases to about 1500 bases, about 1500 bases to about 6000 bases, about 1500 bases to about 5500 bases, about 1500 bases to about 5000 bases, about 1500 bases to about 4500 bases, about 1500 bases to about 4000 bases, about 1500 bases to about 3500 bases, about 1500 bases to about 3000 bases, about 1500 bases to about 2500 bases, about 1500 bases to about 2000 bases, about 2000 bases to about 6000 bases, about 2000 bases to about 5500 bases, about 2000 bases to about 5000 bases, about 2000 bases to about 4500 bases, about 2000 bases to about 4000 bases, about 2000 bases to about 3500 bases, about 2000 bases to about 3000 bases, about 2000 bases to about 2500 bases, about 2500 bases to about 6000 bases, about 2500 bases to about 5500 bases, about 2500 bases to about 5000 bases, about 2500 bases to about 4500 bases, about 2500 bases to about 4000 bases, about 2500 bases to about 3500 bases, about 2500 bases to about 3000 bases, about 3000 bases to about 6000 bases, about 3000 bases to about 5500 bases, about 3000 bases to about 5000 bases, about 3000 bases to about 4500 bases, about 3000 bases to about 4000 bases, about 3000 bases to about 3500 bases, about 3500 bases to about 6000 bases, about 3500 bases to about 5500 bases, about 3500 bases to about 5000 bases, about 3500 to about 4500 bases, about 3500 to about 4000 bases, about 4000 bases to about 6000 bases, about 4000 bases to about 5500 bases, about 4000 bases to about 5000 bases, about 4000 bases to about 4500 bases, about 4500 bases to about 6000 bases, about 4500 bases to about 5500 bases, about 4500 bases to about 5000 bases, about 5000 bases to about 6000 bases, about 5000 bases to about 5500 bases, about 5500 to about 6000 bases, about 24, 30, 40, 50, 60, 70, 80, 90, 100, 200, 250, 500, 750, 1000, 1500, 2000, 2500, 3000, 3500, 4000, 4500, 5000, 5500, or 6000 bases. It should be understood that the length of the first portion of the first strand and the third portion of the second strand are exemplary and that other sizes are within the scope of the present disclosure.

In any of the foregoing embodiments, where a plurality of 5′ adapter is used for 5′ adapter ligation, the second portion of the first strand and the fourth portion of the second strand, i.e. the first 3′ overhang and the second 3′ overhang, respectively, can each include from about 4 bases to about 100 bases. By way of example, but not limitation, the second portion of the first strand and the fourth portion of the second strand can each include from about 4 bases to about 100 bases, about 4 bases to about 90 bases, about 4 bases to about 80 bases, about 4 bases to about 75 bases, about 4 bases to about 70 bases, about 4 bases to about 60 bases, about 4 bases to about 50 bases, about 4 bases to about 40 bases, about 4 bases to about 30 bases, about 4 bases to about 25 bases, about 4 bases to about 20 bases, about 4 bases to about 15 bases, about 4 bases to about 10 bases, about 4 bases to about 5 bases, about 5 bases to about 100 bases, about 5 bases to about 90 bases, about 5 bases to about 80 bases, about 5 bases to about 75 bases, about 5 bases to about 70 bases, about 5 bases to about 60 bases, about 5 bases to about 55 bases, about 5 bases to about 50 bases, about 5 bases to about 40 bases, about 5 bases to about 30 bases, about 5 bases to about 25 bases, about 5 bases to about 20 bases, about 5 bases to about 15 bases, about 5 bases to about 10 bases, about 10 bases to about 100 bases, about 10 bases to about 90 bases, about 10 bases to about 80 bases, about 10 bases to about 75 bases, about 10 bases to about 70 bases, about 10 bases to about 60 bases, about 10 bases to about 50 bases, about 10 bases to about 40 bases, about 10 bases to about 30 bases, about 10 bases to about 25 bases, about 10 bases to about 20 bases, about 10 bases to about 15 bases, about 15 bases to about 100 bases, about 15 bases to about 90 bases, about 15 bases to about 80 bases, about 15 bases to about 75 bases, about 15 bases to about 70 bases, about 15 bases to about 60 bases, about 15 bases to about 50 bases, about 15 bases to about 40 bases, about 15 bases to about 30 bases, about 15 bases to about 25 bases, about 15 bases to about 20 bases, about 20 bases to about 100 bases, about 20 bases to about 90 bases, about 20 bases to about 80 bases, about 20 bases to about 75 bases, about 20 bases to about 70 bases, about 20 bases to about 60 bases, about 20 bases to about 50 bases, about 20 bases to about 40 bases, about 20 bases to about 30 bases, about 20 bases to about 25 bases, about 25 bases to about 100 bases, about 25 bases to about 90 bases, about 25 bases to about 80 bases, about 25 bases to about 75 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 75 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 75 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 75 bases, about 50 bases to about 70 bases, about 50 bases to about 60 bases, about 60 bases to about 100 bases, about 60 bases to about 90 bases, about 60 bases to about 80 bases, about 60 bases to about 75 bases, about 60 bases to about 70 bases, about 70 bases to about 100 bases, about 70 bases to about 90 bases, about 70 bases to about 80 bases, about 70 bases to about 75 bases, about 75 bases to about 100 bases, about 75 bases to about 90 bases, about 75 bases to about 80 bases, about 80 bases to about 100 bases, about 80 bases to about 90 bases, about 90 bases to about 100 bases, about 4, 5, 6, 7, 8, 9, 10, 11, 12, 13, 14, 15, 20, 25, 30, 33, 40, 50, 60, 70, 75, 80, 90, or 100 bases.

In any of the foregoing embodiments, where a plurality of 5′ adapter is used for 5′ adapter ligation, the first common sequence can include from about 1 base to about 50 bases. By way of example, but not limitation, the first common sequence can include from about 1 base to about 50 bases, about 1 base to about 45 bases, about 1 base to about 40 bases, about 1 base to about 35 bases, about 1 base to about 30 bases, about 1 base to about 25 bases, about 1 base to about 20 bases, about 1 base to about 15 bases, about 1 base to about 10 bases, about 5 to about 50 bases, about 5 bases to about 45 bases, about 5 bases to about 40 bases, about 5 bases to about 35 bases, about 5 bases to about 30 bases, about 5 bases to about 25 bases, about 5 bases to about 20 bases, about 5 bases to about 15 bases, about 5 bases to about 10 bases, about 10 bases to about 50 bases, about 10 bases to about 45 bases, about 10 bases to about 40 bases, about 10 bases to about 35 bases, about 10 bases to about 30 bases, about 10 bases to about 25 bases, about 10 bases to about 20 bases, about 10 bases to about 15 bases, about 15 bases to about 50 bases, about 15 bases to about 45 bases, about 15 bases to about 40 bases, about 15 bases to about 35 bases, about 15 bases to about 30 bases, about 15 bases to about 25 bases, about 15 bases to about 20 bases, about 20 bases to about 50 bases, about 20 bases to about 45 bases, about 20 bases to about 40 bases, about 20 bases to about 35 bases, about 20 bases to about 30 bases, about 25 bases to about 30 bases, about 30 bases to about 50 bases, about 30 bases to about 45 bases, about 30 bases to about 40 bases, about 30 bases to about 35 bases, about 35 bases to about 50 bases, about 35 bases to about 45 bases, about 35 bases to about 40 bases, about 40 bases to about 50 bases, about 40 bases to about 45 bases, about 45 bases to about 50 bases, about 1, 2, 3, 4, 5, 6, 7, 8, 9, 10, 11, 12, 13, 14, 15, 20, 25, 30, 33, 35, 40, or 50 bases. Preferably, the first common nucleotide sequence comprises 13 bases. In some embodiments, the first common nucleotide sequence includes the sequence of SEQ ID NO: 127 (5′-AGATCGGAAGAGC-3′). Where the first common nucleotide sequence includes the sequence of SEQ ID NO: 127, the third 3′ terminal portion of each of the plurality of 5′ adapters can each include the sequence of SEQ ID NO: 126 (5′-GCTCTTCCGATCT-3′).

In any of the foregoing embodiments, where a plurality of 5′ adapter is used for 5′ adapter ligation, each of the plurality of first indexing primers can have a length of from about 20 bases to about 100 bases. By way of example, but not limitation, each of the plurality of first indexing primers can have a length from about 20 bases to about 100 bases, about 20 bases to about 90 bases, about 20 bases to about 80 bases, about 20 bases to about 70 bases, about 20 bases to about 60 bases, about 20 bases to about 50 bases, about 20 bases to about 40 bases, about 20 bases to about 30 bases, about 25 bases to about 100 bases, about 25 bases to about 90 bases, about 25 bases to about 80 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 70 bases, about 50 bases to about 60 bases, about 60 bases to about 100 bases, about 60 bases to about 90 bases, about 60 bases to about 80 bases, about 60 bases to about 70 bases, about 70 bases to about 100 bases, about 70 bases to about 90 bases, about 70 bases to about 80 bases, about 80 bases to about 100 bases, about 80 bases to about 90 bases, about 90 bases to about 100 bases, about 30, 35, 40, 45, 50, 60, 70, 80, 90, or 100 bases.

In any of the foregoing embodiments, where a plurality of 5′ adapter is used for 5′ adapter ligation, each of the plurality of second indexing primers can have a length of from about 20 bases to about 100 bases. By way of example, but not limitation, each of the plurality of second indexing primers can have a length from about 20 bases to about 100 bases, about 20 bases to about 90 bases, about 20 bases to about 80 bases, about 20 bases to about 70 bases, about 20 bases to about 60 bases, about 20 bases to about 50 bases, about 20 bases to about 40 bases, about 20 bases to about 30 bases, about 25 bases to about 100 bases, about 25 bases to about 90 bases, about 25 bases to about 80 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 70 bases, about 50 bases to about 60 bases, about 60 bases to about 100 bases, about 60 bases to about 90 bases, about 60 bases to about 80 bases, about 60 bases to about 70 bases, about 70 bases to about 100 bases, about 70 bases to about 90 bases, about 70 bases to about 80 bases, about 80 bases to about 100 bases, about 80 bases to about 90 bases, about 90 bases to about 100 bases, about 30, 35, 40, 45, 50, 60, 70, 80, 90, or 100 bases.

In any of the foregoing embodiments, where a plurality of 5′ adapter is used for 5′ adapter ligation, the first common nucleotide sequence and the third 3′ terminal portion can have a melting temperature greater than the ligation temperature.

In any of the foregoing embodiments, where a plurality of 5′ adapter is used for 5′ adapter ligation, the second 3′ terminal portion of each of the plurality of second indexing primers and the second common nucleotide sequence can have a melting temperature greater than the first annealing temperature. By way of example, but not limitation, the melting temperature of the second common nucleotide sequence and the second 3′ terminal portion can be at least 1° C., 2° C., 3° C., 4° C., 5° C., 6° C., 7° C., 8° C., 9° C., 10° C., 15° C., 20° C., or 25° C. higher than the annealing temperature.

In any of the foregoing embodiments, where a plurality of 5′ adapter is used for 5′ adapter ligation, the melting temperature of the first common nucleotide sequence and the third 3′ terminal portion can be at least 1° C. higher than the ligation temperature. By way of example, but not limitation, the melting temperature of the first common nucleotide sequence and the third 3′ terminal portion can be at least 1° C., 2° C., 3° C., 4° C., 5° C., 6° C., 7° C., 8° C., 9° C., 10° C., 15 ° C., 20° C., or 25° C. higher than the ligation temperature.

In any of the foregoing embodiments, where a plurality of 5′ adapter is used for 5′ adapter ligation, the melting temperature of the first common nucleotide sequence and the third 3′ terminal portion can be at least 1° C. lower than the first annealing temperature. By way of example, but not limitation, the melting temperature of the first common nucleotide sequence and the third 3′ terminal portion can be at least 1° C., 2° C., 3° C., 4° C., 5° C., 6° C., 7° C., 8° C., 9° C., 10° C., 15° C., 20° C., or 25° C. lower than the first annealing temperature.

In any of the foregoing embodiments, where a plurality of 5′ adapter is used for 5′ adapter ligation, the second common nucleotide sequence can have a length from about 3 bases to about 100 bases. By way of example, but not limitation, the second common nucleotide sequence can have a length from about 1 base to about 100 bases, about 1 base to about 90 bases, about 1 base to about 80 bases, about 1 base to about 75 bases, about 1 base to about 70 bases, about 1 base to about 60 bases, about 1 base to about 50 bases, about 1 base to about 40 bases, about 1 base to about 30 bases, about 1 base to about 25 bases, about 1 base to about 20 bases, about 1 base to about 15 bases, about 1 base to about 10 bases, about 1 base to about 5 bases, about 5 bases to about 100 bases, about 5 bases to about 90 bases, about 5 bases to about 80 bases, about 5 bases to about 75 bases, about 5 bases to about 70 bases, about 5 bases to about 60 bases, about 5 bases to about 50 bases, about 5 bases to about 40 bases, about 5 bases to about 30 bases, about 5 bases to about 25 bases, about 5 bases to about 20 bases, about 5 bases to about 15 bases, about 5 bases to about 10 bases, about 10 bases to about 100 bases, about 10 bases to about 90 bases, about 10 bases to about 80 bases, about 10 bases to about 75 bases, about 10 bases to about 70 bases, about 10 bases to about 60 bases, about 10 bases to about 50 bases, about 10 bases to about 40 bases, about 10 bases to about 30 bases, about 10 bases to about 25 bases, about 10 bases to about 20 bases, about 10 bases to about 15 bases, about 15 bases to about 100 bases, about 15 bases to about 90 bases, about 15 bases to about 80 bases, about 15 bases to about 75 bases, about 15 bases to about 70 bases, about 15 bases to about 60 bases, about 15 bases to about 50 bases, about 15 bases to about 40 bases, about 15 bases to about 30 bases, about 15 bases to about 25 bases, about 15 bases to about 20 bases, about 20 bases to about 100 bases, about 20 bases to about 90 bases, about 20 bases to about 80 bases, about 20 bases to about 75 bases, about 20 bases to about 70 bases, about 20 bases to about 60 bases, about 20 bases to about 50 bases, about 20 bases to about 40 bases, about 20 bases to about 30 bases, about 20 bases to about 25 bases, about 25 bases to about 100 bases, about 25 bases to about 90 bases, about 25 bases to about 80 bases, about 25 bases to about 75 bases, about 25 bases to about 70 bases, about 25 bases to about 60 bases, about 25 bases to about 50 bases, about 25 bases to about 40 bases, about 25 bases to about 30 bases, about 30 bases to about 100 bases, about 30 bases to about 90 bases, about 30 bases to about 80 bases, about 30 bases to about 75 bases, about 30 bases to about 70 bases, about 30 bases to about 60 bases, about 30 bases to about 50 bases, about 30 bases to about 40 bases, about 40 bases to about 100 bases, about 40 bases to about 90 bases, about 40 bases to about 80 bases, about 40 bases to about 75 bases, about 40 bases to about 70 bases, about 40 bases to about 60 bases, about 40 bases to about 50 bases, about 50 bases to about 100 bases, about 50 bases to about 90 bases, about 50 bases to about 80 bases, about 50 bases to about 75 bases, about 50 bases to about 70 bases, about 50 bases to about 60 bases, about 60 bases to about 100 bases, about 60 bases to about 90 bases, about 60 bases to about 80 bases, about 60 bases to about 75 bases, about 60 baes to about 70 bases, about 70 bases to about 100 bases, about 70 bases to about 90 bases, about 70 bases to about 80 bases, about 70 bases to about 75 bases, about 75 bases to about 100 bases, about 75 bases to about 90 bases, about 75 bases to about 80 bases, about 80 bases to about 100 bases, about 80 bases to about 90 bases, about 90 bases to about 100 bases, about 1, 2, 3, 4, 5, 6, 7, 8, 9, 10, 15, 20, 25, 30, 40, 50, 60, 75, 80, 90, or 100 bases.

In any of the foregoing embodiments, where a plurality of 5′ adapter is used for 5′ adapter ligation, the ligation temperature can be less than a melting temperature of the partially double-stranded DNA substrate.

In any of the foregoing embodiments, where a plurality of 5′ adapter is used for 5′ adapter ligation, a melting temperature of the third strand and fourth strand can be less than the first denaturing temperature.

In any of the foregoing embodiments, where a plurality of 5′ adapter is used for 5′ adapter ligation, the ligation temperature can be about 25° C. to about 40° C. By way of further example, but not limitation, the ligation temperature can be about 25° C. to about 40° C., about 25° C. to about 37° C., about 25° C. to about 35° C., about 25° C. to about 30° C., about 30° C. to about 40° C., about 30° C. to about 35° C., about 35° C. to about 40° C., about 25° C., 26° C., 27° C., 28° C., 29° C., 30° C., 31° C., 32° C., 33° C., 34° C., 35° C., 36° C., 37° C., 38° C., 39° C., or 40° C.

In any of the foregoing embodiments, where a plurality of 5′ adapter is used for 5′ adapter ligation, the ligation duration and be any time sufficient to ligate one of the plurality of first indexing primers to the first 5′ end of the first strand or the second 5′ end of the second strand. In any of the foregoing embodiments, the ligation duration can be from about 5 minutes to about 60 minutes. By way of example, but not limitation, the ligation duration can be about 5 minutes to about 60 minutes, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 60 minutes, about 10 minutes to about 50 minutes, about 10 minutes to about 40 minutes, about 10 minutes to about 30 minutes, about 10 minutes to about 20 minutes, about 20 minutes to about 60 minutes, about 20 minutes to about 50 minutes, about 20 minutes to about 40 minutes, about 20 minutes to about 30 minutes, about 30 minutes to about 60 minutes, about 30 minutes to about 50 minutes, about 30 minutes to about 40 minutes, about 40 minutes to about 60 minutes, about 40 minutes to about 50 minutes, about 50 minutes to about 60 minutes, about 5, 10, 15, 20, 25, 30, 40, 50, or 60 minutes.

In any of the foregoing embodiments, where a plurality of 5′ adapter is used for 5′ adapter ligation, any suitable ligase can be used. It should be understood that the ligase should can be a ligase that is a thermolabile ligase capable of ligation in low magnesium buffer and be inactivated at the first denaturation temperature for the first denaturation duration. It should be understood that such low magnesium buffer conditions are those suitable for PCR. By way of example, but not limitation, the ligase can be T3 DNA ligase. In any of the foregoing embodiments, by way of example, but not limitation, the ligase can be added at about 30 to about 300 enzyme units per μL of the first reaction mixture.

In any of the foregoing embodiments, where a plurality of 5′ adapter is used for 5′ adapter ligation, any suitable polymerase can be used. In some embodiments, the polymerase is not active at the ligation temperature. By way of example, but not limitation, the polymerase can further include a hot start antibody or aptamer. In any of the foregoing embodiments, the polymerase can be a hot start polymerase with an activation temperature, where the activation temperature is less than the first denaturation temperature. By way of example, but not limitation, the activation temperature can be less than the first denaturation temperature, second denaturation temperature, and third denaturation temperature. By way of example but not limitation, the polymerase can be can be selected from the group consisting of Kapa HiFi DNA Polymerase (Roche), NEB Q5 DNA Polymerase (NEB), PrimeStar GXL DNA Polymerase (Takara), or High Fidelity DNA Polymerase (Qiagen). In any of the foregoing embodiments, the DNA polymerase can further include a hot start antibody or aptamer that increases the activation temperature of the DNA polymerase. In such embodiments, the DNA polymerase can be Kapa HiFi Hot Start DNA Polymerase (Roche), NEB Q5 Hot Start DNA Polymerase (NEB), PrimeStar GXL Hot Start DNA Polymerase (Takara), or High Fidelity Hot Start DNA Polymerase (Qiagen).

In any of the foregoing embodiments, where a plurality of 5′ adapter is used for 5′ adapter ligation, the first denaturation temperature, the second denaturation temperature, the third denaturation temperature and the fourth denaturation temperature can each be from about 95° C. to about 98° C. In any of the foregoing embodiments, where a plurality of 5′ adapter is used for 5′ adapter ligation, the first denaturation duration, the second denaturation duration, the third denaturation duration and the fourth denaturation duration can each be from about 30 seconds to about 2 minutes. By way of example, but not limitation, the first denaturation duration, the second denaturation duration, the third denaturation duration and the fourth denaturation duration can each be from about 30 seconds to about 2 minutes, about 30 seconds to 1.5 minutes, about 30 seconds to 1 minute, about 1 minute to about 2 minutes, about 1 minutes to about 1.5 minutes, about 1.5 minutes to about 2 minutes, about 30 seconds, 45 seconds, 1 minutes, 1.5 minutes, or 2 minutes. It should be understood that any suitable temperature for denaturing double-stranded DNA to be annealed to in each PCR cycle can be used. It should be understood that the first denaturation temperature should be sufficient that it is higher than a melting temperature of the first strand and second strand. One of skill in the art can readily determine such temperature and the necessary time through routine experimentation and/or knowledge in the art.

In any of the foregoing embodiments, where a plurality of 5′ adapter is used for 5′ adapter ligation, the first annealing temperature, the second annealing temperature, the third annealing temperature, and the fourth annealing temperature can each be from about 55° C. to about 65° C. By way of example, but not limitation, the first annealing temperature, the second annealing temperature, and the third annealing temperature can each be from about 55° C. to about 65° C., 55° C. to about 60° C. , about 60° C. to about 65° C., about 55° C., 56° C., 57° C., 58° C., 59° C., 60° C., 61° C., 62° C., 63° C., 64° C., or 65° C.

In any of the foregoing embodiments, where a plurality of 5′ adapter is used for 5′ adapter ligation, the first annealing duration, the second annealing duration, the third annealing duration, and the fourth annealing duration can each be from about 10 seconds to about 60 seconds. It should be understood that the first annealing temperature, the second annealing temperature, and the third annealing temperature and the first annealing duration, the second annealing duration, and the third annealing duration should be sufficient for the annealing required in each respective PCR step to occur. One of skill in the art can readily determine such temperature and the necessary time through routine experimentation and/or knowledge in the art.

In any of the foregoing embodiments, where a plurality of 5′ adapter is used for 5′ adapter ligation, the first extension temperature, the second extension temperature, the third extension temperature, and the fourth extension temperature can each be from about 60° C. to about 72° C. By way of example, but not limitation, the first extension temperature, the second extension temperature, and the third extension temperature can be about 60° C., 61° C., 62° C., 63° C., 64° C., 65° C., 66° C., 67° C., 68° C., 69° C., 70° C., 71° C., or 72° C.

In any of the foregoing embodiments, where a plurality of 5′ adapter is used for 5′ adapter ligation, the first extension duration, the second extension duration, the third extension duration, and the fourth extension duration can each be from about 30 seconds to about 5 minutes. It should be understood that the first extension temperature, the second extension temperature, and the third extension temperature and the first extension duration, the second extension duration, and the third extension duration should be sufficient for the extension required in each respective PCR step to occur. One of skill in the art can readily determine such temperature and the necessary time through routine experimentation and/or knowledge in the art.

In any of the foregoing embodiments, where a plurality of 5′ adapter is used for 5′ adapter ligation, the plurality of first indexing primers and the plurality of second indexing primers can be added to the first reaction mixture at about 100 nM to about 1 μM. By way of example, but not limitation, the plurality of first indexing primers and the plurality of second indexing primers can be added to the first reaction mixture at about 100 nM to about 200 nM.

In any of the foregoing embodiments, where a plurality of 5′ adapter is used for 5′ adapter ligation, each of the plurality of first indexing primers can further include the sequence of SEQ ID NO: 87. In any of the foregoing embodiments, each of the plurality of second indexing primers can further include the sequence of SEQ ID NO: 78.

In any of the foregoing embodiments, where a plurality of 5′ adapter is used for 5′ adapter ligation, the method can further include sequencing the seventh strand and the ninth strand or the eighth strand and the tenth strand.

In any of the foregoing embodiments, where a plurality of 5′ adapter is used for 5′ adapter ligation,

In any of the foregoing embodiments, it should be understood that the third 3′ terminal portion and the first common nucleotide sequence can have a Tₘ higher than the ligation temperature, but lower than the first annealing temperature, the second annealing temperature, the third annealing temperature and the fourth annealing temperature.

In any of the foregoing embodiments for ligation-coupled PCR, it should be understood that each of the plurality of first indexing primers can be not complementary to the second common nucleotide sequence.

In any of the foregoing embodiments for ligation-coupled PCR, where a strand is recited as comprising certain elements, it should be understood that the strand can include additional unrecited elements. By way of example, but not limitation, in the embodiments that utilize a 5′ hairpin adapter, the from the fifth strand on can include the first common nucleotide sequence and a reverse complement of the first common nucleotide sequence. Thus, it should be understood that by not reciting these elements, they are not excluded by omission.

Methods for Generating the Partially Double-Stranded DNA Substrate

It should be understood in methods of the present disclosure that instead of starting from a partially double-stranded DNA substrate as described, the methods can be modified to start with a starting DNA substrate molecule which can be processed during the ligation step, e.g. step (ii), to yield the partially double-stranded DNA substrate. By way of example, but not limitation, steps (i) and (ii) can be combined such that a starting DNA substrate molecule can be provided, combined with the reagents for the first reaction mixture in addition to the treatments and enzymes disclosed herein for preparing the partially double-stranded DNA substrate. In such embodiments, the first set of conditions can also be sufficient for the enzymes, e.g. UDG and Endonuclease VIII, RNase H or Endonuclease V to cleave dU bases, ribonucleotides or inosine, bases respectively during the ligation step (ii). In step (iii), the second set of conditions can be further sufficient to inactivate any such enzymes so that they do not affect further PCR amplifaction.

In some embodiments, the partially double-stranded DNA substrate can be produced by: providing a starting double-stranded DNA substrate molecule comprising a first starting strand and a second starting strand each comprising a universal primer sequence at their 5′ ends, where the universal primer sequence includes dU bases internal to or at a 3′ end of the universal primer sequence, adding an enzyme capable of cleaving the dU bases, and incubating the enzyme capable of cleaving the dU bases and the starting double-stranded DNA substrate molecule under conditions sufficient to yield the partially double-stranded DNA substrate of step (i). By way of example, but not limitation, the enzyme capable of cleaving the dU bases can be a combination of UDG and Endonuclease VIII.

In some embodiments, the partially double-stranded DNA substrate can be produced by: providing a starting double-stranded DNA substrate molecule comprising a first starting strand and a second starting strand, the first starting strand including a first 5′ cleavable sequence, the second starting strand including a second 5′ cleavable sequence, where each of the first 5′ cleavable sequence and second 5′ cleavable sequence comprise ribonucleotides internal to or at a 3′ end of the first 5′ cleavable sequence and second 5′ cleavable sequence, adding an enzyme capable of cleaving the ribonucleotides, and incubating the enzyme capable of cleaving the ribonucleotides and the starting double-stranded DNA substrate molecule under conditions sufficient to yield the partially double-stranded DNA substrate of step (i). By way of example, but not limitation, the enzyme capable of cleaving the ribonucleotides is RNase H.

In some embodiments, the partially double-stranded DNA substrate can be produced by: providing a starting double-stranded DNA substrate molecule comprising a first starting strand and a second starting strand, the first starting strand including a first 5′ cleavable sequence, the second starting strand including a second 5′ cleavable sequence, where each of the first 5′ cleavable sequence and second 5′ cleavable sequence comprise inosine bases internal to or at a 3′ end of the first 5′ cleavable sequence and second 5′ cleavable sequence, adding an enzyme capable of cleaving the inosine bases, and incubating the enzyme capable of cleaving the inosine bases and the starting double-stranded DNA substrate molecule under conditions sufficient to yield the partially double-stranded DNA substrate of step (i). By way of example, but not limitation, the enzyme capable of cleaving the inosine bases is Endonuclease V.

In some embodiments, the partially double-stranded DNA substrate can be produced by: providing a fragmented DNA substrate molecule, performing end repair to yield a blunt-ended, double-stranded starting DNA substrate molecule which has a first starting strand a a second starting strand each with a 5′ end a 3′ end, adding a plurality of 3′ adapter strands where each 3′ adapter strand is annealed to a complementary strand which includes dU bases and a ligase to the blunt-ended, double stranded starting DNA molecule, incubating the plurality of 3′ adapter strands annealed to complementary strands including dU bases, the ligase, and the blunt-ended double-stranded DNA substrate molecule under conditions sufficient to ligate one of the plurality of 3′ adapter strands to each 3′ end of the first starting strand and the second starting strand of the blunt-ended starting DNA substrate molecule and leave a nick between each complementary strand and each 5′ end of the first starting strand and second starting strand to yield a ligated, double-stranded substrate molecule, optionally purifying the ligated, double-stranded molecule, adding to the ligated, double-stranded substrate molecule an enzyme capable of cleaving the dU bases of the ligated, double-stranded molecule, and incubating the enzyme capable of cleaving the dU bases and the ligated, double-stranded substrate molecule under conditions sufficient to cleave the complementary strands to yield the partially double-stranded DNA substrate. By way of example, but not limitation, the enzyme capable of cleaving the dU bases can be a combination of UDG and Endonuclease VIII.

In some embodiments, the partially double-stranded DNA substrate can be produced by: providing a fragmented DNA substrate molecule, performing end repair to yield a blunt-ended, double-stranded starting DNA substrate molecule which has a first starting strand a a second starting strand each with a 5′ end a 3′ end, adding a plurality of 3′ adapter strands where each 3′ adapter strand is annealed to a complementary strand which includes ribonucleotides and a ligase to the blunt-ended, double stranded starting DNA molecule, incubating the plurality of 3′ adapter strands annealed to complementary strands including dU bases, the ligase, and the blunt-ended double-stranded DNA substrate molecule under conditions sufficient to ligate one of the plurality of 3′ adapter strands to each 3′ end of the first starting strand and the second starting strand of the blunt-ended starting DNA substrate molecule and leave a nick between each complementary strand and each 5′ end of the first starting strand and second starting strand to yield a ligated, double-stranded substrate molecule, optionally purifying the ligated, double-stranded molecule, adding to the ligated, double-stranded substrate molecule an enzyme capable of cleaving the ribonucleotides of the ligated, double-stranded molecule, and incubating the enzyme capable of cleaving the ribonucleotides and the ligated, double-stranded substrate molecule under conditions sufficient to cleave the complementary strands to yield the partially double-stranded DNA substrate. By way of example, but not limitation, the enzyme capable of cleaving the ribonucleotides is RNase H.

In some embodiments, the partially double-stranded DNA substrate can be produced by: providing a fragmented DNA substrate molecule, performing end repair to yield a blunt-ended, double-stranded starting DNA substrate molecule which has a first starting strand a a second starting strand each with a 5′ end a 3′ end, adding a plurality of 3′ adapter strands where each 3′ adapter strand is annealed to a complementary strand which includes inosine bases and a ligase to the blunt-ended, double stranded starting DNA molecule, incubating the plurality of 3′ adapter strands annealed to complementary strands including inosine bases, the ligase, and the blunt-ended double-stranded DNA substrate molecule under conditions sufficient to ligate one of the plurality of 3′ adapter strands to each 3′ end of the first starting strand and the second starting strand of the blunt-ended starting DNA substrate molecule and leave a nick between each complementary strand and each 5′ end of the first starting strand and second starting strand to yield a ligated, double-stranded substrate molecule, optionally purifying the ligated, double-stranded molecule, adding to the ligated, double-stranded substrate molecule an enzyme capable of cleaving the inosine bases of the ligated, double-stranded molecule, and incubating the enzyme capable of cleaving the inosine bases and the ligated, double-stranded substrate molecule under conditions sufficient to cleave the complementary strands to yield the partially double-stranded DNA substrate. By way of example, but not limitation, the enzyme capable of cleaving the inosine bases is Endonuclease V.

In some embodiments, the partially double-stranded DNA substrate can be produced by: providing a fragmented DNA substrate molecule; performing end repair and A-tailing of the fragmented DNA substrate molecule to yield a single-A-tailed double-stranded starting DNA substrate molecule comprising a first starting strand and a second starting strand, the single-A-tailed double-stranded starting DNA substrate molecule comprising a single A base overhang at each 3′ end; adding a plurality of 3′ adapter strands, each 3′ adapter strand annealed to a complementary strand comprising a 3′ terminal dU base and a ligase to the single-A-tailed double-stranded starting DNA substrate molecule; incubating the plurality of 3′ adapter strands annealed to complementary strands comprising a 3′ terminal dU base, the ligase, and the single-A-tailed double-stranded starting DNA substrate molecule under conditions sufficient to ligate one of the plurality of 3′ adapter strands to each single A base overhang and to ligate one of the complementary strands to each 5′ end of the single-A-tailed double-stranded starting DNA substrate molecule, to yield a ligated, double-stranded substrate molecule; optionally purifying the ligated, double-stranded substrate molecule; adding an enzyme capable of cleaving the dU bases to the ligated, double-stranded substrate molecule; and incubating the enzyme capable of cleaving the dU bases and the ligated, double-stranded substrate molecule under conditions sufficient to cleave the complementary strands to yield the partially double-stranded DNA substrate of step (i).

Alternatively, in the immediately foregoing embodiment, the complementary strand can comprise a 3′ terminal T base instead of a 3′ terminal dU base, but further comprise dU bases. In such instances, the ligation can still occur, however, a portion of the complementary strand will remain after endonuclease cleavage. Further, alternatively, while having the 3′ terminal T base, the complementary strand can include ribonucleotides or inosine bases, and the enzyme added can be capable of cleaving the riboucleotides or inosine bases, respectively.

In any of the foregoing methods where a fragmented DNA substrate molecule is provided, the method can further include fragmenting target DNA to yield the fragmented DNA substrate molecule.

In any of the foregoing embodiments, where a polymerase is used, such as for multiplex PCR, the DNA polymerase can be any suitable polymerase. In some embodiments, the polymerase can be a uracil tolerant DNA polymerase such a high fidelity DNA polymerase.

It should be understood that the ligase and, if used, polymerase, for generating the partially double-stranded DNA substrate can be different or the same as the ligase and polymerase used for the ligated-coupled PCR.

In any of the foregoing embodiments, where a plurality of 3′ adapter strands are used, each of the plurality of 3′ adapter strands can further include a 5′ phosphate and the complementary strand can further include a 3′ blocking group. By way of example, but not limitation, the 3′ blocking group can be selected from the group consisting of 3′-deoxythymidine, 3′-deoxyadenine, 3′-deoxyguanine, 3′-deoxycytosine, and a 2′3′-dideoxy nucleotide.

Library Normalization After Ligation-Coupled PCR

In any of the foregoing embodiments, after the PCR cycles, library normalization can be performed.

In any of the foregoing embodiments where each of the plurality of first indexing primers has a first 3′ terminal portion complementary to the first common nucleotide sequence and each of the plurality of second indexing primers has a second 3′ terminal portion complementary to the first common nucleotide sequence, each of the plurality of first indexing primers comprises a first 5′ terminal portion and each of the plurality of second indexing primers further comprises a second 5′ terminal portion, wherein each of the first 5′ terminal portion and the second 5′ terminal portion comprise, in a 5′ to 3′ direction, a first sequence comprising two or more deoxynucleotides and a second sequence comprising three or more ribonucleotides, wherein the DNA polymerase has 3′ to 5′ exonuclease activity, whereby the fifth strand and sixth strand further comprise the second 5′ terminal portion at a 5′ end of the fifth strand and sixth strand, and whereby the seventh strand and eighth strand further comprise the first 5′ terminal portion at a 5′ end of the seventh strand and eighth strand, and whereby the fifth strand and seventh strand can form a first double-stranded product having a first 5′ overhang and a second 5′ overhang, and whereby the sixth strand and eighth strand can form a second double-stranded product having a third 5′ overhang and a fourth 5′ overhang, the method further comprising: adding a probe complementary to each of the first 5′ overhang, second 5′ overhang, third 5′ overhang and fourth 5′ overhang in an amount sufficient to yield a target molar quantity of the fifth strand, sixth strand, seventh strand and eighth strand, where the fifth strand, sixth strand, seventh strand and eighth strand are present in an amount greater than the target molar quantity, and a second ligase; incubating the fifth strand, sixth strand, seventh strand, eighth strand, second ligase and probe under conditions sufficient for the probe to ligate to the target molar quantity of the fifth strand, sixth strand, seventh strand and eighth strand to yield a pre-normalization reaction mixture; adding an enzyme with exonuclease activity to the pre-normalization reaction mixture; incubating the pre-normalization reaction mixture and enzyme with exonuclease activity under conditions sufficient for the enzyme with exonuclease activity to digest the fifth strand sixth strand, seventh strand, and eighth strand not ligated to the probe, to yield a normalized next generation sequencing (NGS) library. By way of example, but not limitation, the second ligase can be T4 DNA second ligase.

In any of the foregoing embodiments, where a plurality of 5′ adapter is used for 5′ adapter ligation, the method can further include wherein each of the plurality of first indexing primers comprises a first 5′ terminal portion and each of the plurality of second indexing primers further comprises a second 5′ terminal portion, wherein each of the first 5′ terminal portion and the second 5′ terminal portion comprise, in a 5′ to 3′ direction, a first sequence comprising two or more deoxynucleotides and a second sequence comprising three or more ribonucleotides, wherein the DNA polymerase has 3′ to 5′ exonuclease activity, whereby the seventh strand and eighth strand further comprise the first 5′ terminal portion at a 5′ end of the seventh strand and eighth strand, and whereby the ninth strand and tenth strand further comprise the second 5′ terminal portion at a 5′ end of the ninth strand and tenth strand, and whereby the seventh strand and ninth strand can form a first double-stranded product having a first 5′ overhang and a second 5′ overhang, and whereby the eighth strand and tenth strand can form a second double-stranded product having a third 5′ overhang and a fourth 5′ overhang, the method further comprising: adding a probe complementary to each of the first 5′ overhang, second 5′ overhang, third 5′ overhang and fourth 5′ overhang in an amount sufficient to yield a target molar quantity of the seventh strand, eighth strand, ninth strand, and tenth strand, and a second ligase where the seventh strand, eighth strand, ninth strand, and tenth strand are present in an amount greater than the target molar quantity; incubating the seventh strand, eighth strand, ninth strand, and tenth strand, second ligase and probe under conditions sufficient for the probe to ligate to the target molar quantity of the seventh strand, eighth strand, ninth strand, and tenth strand to yield a pre-normalization reaction mixture; adding an enzyme with exonuclease activity to the pre-normalization reaction mixture; incubating the pre-normalization reaction mixture and enzyme with exonuclease activity under conditions sufficient for the enzyme with exonuclease activity to digest the seventh strand, eighth strand, ninth strand, and tenth strand not ligated to the probe, to yield a normalized next generation sequencing (NGS) library.

It should be understood that in the foregoing embodiments for normalization, the enzyme with exonuclease activity can be Exonuclease III. It should also be further understood in the foregoing embodiments for normalization, the probe includes a modification to provide resistance to exonuclease digestion by an enzyme with 3′ exonuclease activity, such as, by way of example but not limitation, a phosphorothioate linkage.

Methods for Splint-Mediated Primer Assembly

Methods for Splint-Mediated Primer Assembly (One Primer)

In some embodiments, a method for ligation-coupled PCR is provided that can include: (i) providing a double-stranded DNA substrate comprising a first strand and a second strand, the first strand comprising a first 3′ terminal portion and a first 5′ terminal portion and the second strand comprising a second 3′ terminal portion and a second 5′ terminal portion; (ii) adding a ligase, a first oligonucleotide, a second oligonucleotide, a third oligonucleotide, a first primer, a DNA polymerase and deoxynucleotide triphosphates (dNTPs) to the double-stranded DNA substrate to yield a first reaction mixture, the first oligonucleotide comprises a third 3′ terminal portion complementary to at least a portion of the first 3′ terminal portion of the first strand and a 5′ phosphate, the third oligonucleotide comprises a first 3′ blocking group, the first primer comprising a fourth 3′ terminal portion that is complementary to at least a portion of the second 3′ terminal portion of the second strand, wherein the first 3′ blocking group is not competent for polymerase chain extension, wherein the second oligonucleotide has a 3′ portion complementary a 3′ portion of the third oligonucleotide and the first oligonucleotide has a 5′ portion complementary to a 5′ portion of the third oligonucleotide; (iii) incubating the first reaction mixture under a first set of conditions comprising a ligation temperature for a ligation duration sufficient for the first oligonucleotide and the second oligonucleotide to anneal to the third oligonucleotide in a 3′-5′ direction to yield a second reaction mixture comprising the first primer, the double-stranded DNA substrate, and a second primer comprising the first oligonucleotide ligated to the second oligonucleotide; and (iv) subjecting the second reaction mixture to at least 3 PCR cycles, wherein each cycle independently comprises a denaturation temperature for a denaturation duration, an annealing temperature for an annealing duration, and an extension temperature for an extension duration, to yield a third reaction mixture comprising a double-stranded library molecule comprising the second primer at a first 5′ end of the double-stranded library molecule and comprising the first primer at a second 5′ end of the double-stranded library molecule.

In any of the foregoing embodiments, for splint-mediated primer assembly, step (ii) can further include adding a fourth oligonucleotide and a fifth oligonucleotide, wherein the second oligonucleotide further comprises a 5′ phosphate, wherein the fifth oligonucleotide comprises a second 3′ blocking group not competent for polymerase chain extension, wherein the fifth oligonucleotide further comprises a 5′ portion complementary to a 5′ portion of the second oligonucleotide and a 3′ portion complementary to a 3′ portion of the fourth oligonucleotide, wherein the first set of conditions is further sufficient for the second oligonucleotide and the fourth oligonucleotide to anneal to the fifth oligonucleotide and for the ligase to ligate the fourth oligonucleotide to the second oligonucleotide, thereby yielding the second primer comprising, in a 5′ to 3′ direction, the fourth oligonucleotide, the second oligonucleotide, and the first oligonucleotide. In such embodiments, the ligation temperature can be less than the melting temperature of the second oligonucleotide and fifth oligonucleotide and less than the melting temperature of the fourth oligonucleotide and fifth oligonucleotide. In such embodiments, the melting temperature of the fifth oligonucleotide, fourth oligonucleotide and second oligonucleotide can be lower than the annealing temperature and the extension temperature.

In any of the foregoing embodiments, for splint-mediated primer assembly, the first oligonucleotide, second oligonucleotide and third oligonucleotide can have a melting temperature lower than the annealing temperature and the extension temperature.

Methods for Splint-Mediated Primer Assembly (Both Primers)

In some embodiments, a method for ligation-coupled PCR can include (i) providing a double-stranded DNA substrate comprising a first strand and a second strand, the first strand comprising a first 3′ terminal portion and a first 5′ terminal portion and the second strand comprising a second 3′ terminal portion and a second 5′ terminal portion; (ii) adding a ligase, a first oligonucleotide, a second oligonucleotide, a third oligonucleotide, a fourth oligonucleotide, a fifth oligonucleotide, a sixth oligonucleotide, a DNA polymerase and deoxynucleotide triphosphates (dNTPs) to the double-stranded DNA substrate to yield a first reaction mixture, the first oligonucleotide comprises a third 3′ terminal portion complementary to at least a portion of the first 3′ terminal portion of the first strand and a 5′ phosphate, the third oligonucleotide comprises a first 3′ blocking group, wherein the second oligonucleotide has a 3′ portion complementary a 3′ portion of the third oligonucleotide and the first oligonucleotide has a 5′ portion complementary to a 5′ portion of the third oligonucleotide, the fourth oligonucleotide comprises a fourth 3′ terminal portion complementary to at least a portion of the second 3′ terminal portion of the second strand and a 5′ phosphate, the sixth oligonucleotide comprises a 5′ portion complementary to a 5′ portion of the fourth oligonucleotide, a 3′ portion complementary to a 3′ portion of the fifth oligonucleotide, and a second 3′ blocking group, wherein the first 3′ blocking group and the second 3′ blocking group are not competent for polymerase chain extension; (iii) incubating the first reaction mixture under a first set of conditions comprising a ligation temperature for a ligation duration sufficient for the first oligonucleotide and the second oligonucleotide to anneal to the third oligonucleotide in a 3′-5′ direction and for the fourth oligonucleotide and fifth oligonucleotide to anneal to the sixth oligonucleotide in a 3′-5′ direction, to yield a second reaction mixture comprising a first primer comprising the fourth oligonucleotide ligated to the fifth oligonucleotide, the double-stranded DNA substrate, and a second primer comprising the first oligonucleotide ligated to the second oligonucleotide; (iv) subjecting the second reaction mixture to at least 3 PCR cycles, wherein each cycle independently comprises a denaturation temperature for a denaturation duration, an annealing temperature for an annealing duration, and an extension temperature for an extension duration, to yield a third reaction mixture comprising a double-stranded library molecule comprising the second primer at a first 5′ end of the double-stranded library molecule and comprising the first primer at a second 5′ end of the double-stranded library molecule.

In any of the foregoing embodiments for splint-mediated primer assembly of both primers, the melting temperature of the first oligonucleotide, second oligonucleotide and third oligonucleotide can be lower than the annealing temperature and the extension temperature, and the melting temperature of the fourth, fifth and sixth oligonucleotides can be lower than the annealing temperature and the extension temperate. It should be understood that in such embodiments, the splint oligonucleotides—in these embodiments, the third oligonucleotide and fifth oligonucleotide—can thus be denatured from the assembled, ligated primers. Thus, the melting temperature can refer to the melting temperature of the splint to the ligated oligonucleotides.

In any of the foregoing embodiments for splint-mediated primer assembly of both primers, step (ii) can further include adding a seventh oligonucleotide and an eighth oligonucleotide, wherein the second oligonucleotide further comprises a 5′ phosphate, wherein the eighth oligonucleotide comprises a third 3′ blocking group not competent for polymerase chain extension, wherein the eighth oligonucleotide further comprises a 3′ portion complementary to a 3′ portion of the seventh oligonucleotide and a 5′ portion complementary to the second oligonucleotide, wherein the first set of conditions is further sufficient for the seventh oligonucleotide and the second oligonucleotide to anneal to the eighth oligonucleotide and for the ligase to ligase the seventh oligonucleotide to the second oligonucleotide, thereby yielding the second primer comprising, in a 5′ to 3′ direction, the seventh oligonucleotide, the second oligonucleotide, and the first oligonucleotide. In such embodiments, the ligation temperature can be less than a melting temperature of the seventh oligonucleotide and second oligonucleotide to the eighth oligonucleotide. In such embodiments, the annealing temperature and extension temperature can be greater than the melting temperature of the seventh oligonucleotide and second oligonucleotide to the eighth oligonucleotide.

In any of the foregoing embodiments for splint-mediated primer assembly of one or both primers, the oligonucleotides to be ligated can each have a length from about 5 to about 100 bases while the oligonucleotides having the 3′ blocking group can have a length from about 10 to about 50 bases.

In any of the foregoing embodiments for splint-mediated primer assembly, the method can further include (v) subjecting the third reaction mixture to additional PCR cycles to amplify the double-stranded library molecule.

In any of the foregoing embodiments for splint-mediated primer assembly, the ligation temperature can be less than a melting temperature of the double-stranded DNA substrate.

In any of the foregoing embodiments for splint-mediated primer assembly, the ligation temperature can be less than a melting temperature of the first oligonucleotide and the third oligonucleotide, and the ligation temperature can be less than a melting temperature of the second oligonucleotide and third oligonucleotide. By way of further example, but not limitation, in any of the foregoing embodiments for single splint-mediated primer assembly, the ligation temperature can be less than a melting temperature of the fourth oligonucleotide and the fifth oligonucleotide. By way of still further example, but not limitation, in any of the foregoing embodiments for splint-mediated primer assembly of both primers, the ligation temperature can also be less than the melting temperature of the fourth oligonucleotide and sixth oligonucleotide and the fifth oligonucleotide and sixth oligonucleotide.

In any of the foregoing embodiments for splint-mediated primer assembly, any suitable ligase can be used. It should be understood that the ligase should can be a ligase that is a thermolabile ligase capable of ligation in low magnesium buffer and be inactivated at the first denaturation temperature for the first denaturation duration. It should be understood that such low magnesium buffer conditions are those suitable for PCR. By way of example, but not limitation, the ligase can be T3 DNA ligase. In any of the foregoing embodiments, by way of example, but not limitation, the ligase can be added at about 30 to about 300 enzyme units per μL of the first reaction mixture

In any of the foregoing embodiments for splint-mediated primer assembly, any of the 3′ blocking groups can be independently selected form the group consisting of a C3 spacer, hexanediol, Spacer 9, Spacer 18, three or more rU bases, a phosphate, and 2′-O-methyl bases.

In any of the foregoing embodiments for splint-mediated primer assembly, steps (i)-(iv) can be performed in the same tube.

In any of the foregoing embodiments for splint-mediated primer assembly, no purification can be performed between steps (iii) and (iv).

In any of the foregoing embodiments for splint-mediated primer assembly, any suitable polymerase can be used. In some embodiments, the polymerase is not active at the ligation temperature. By way of example, but not limitation, the polymerase can further include a hot start antibody or aptamer. In any of the foregoing embodiments, the polymerase can be a hot start polymerase with an activation temperature, where the activation temperature is less than the first denaturation temperature. By way of example, but not limitation, the activation temperature can be less than the first denaturation temperature, second denaturation temperature, and third denaturation temperature. By way of example but not limitation, the polymerase can be can be selected from the group consisting of Kapa HiFi DNA Polymerase (Roche), NEB Q5 DNA Polymerase (NEB), PrimeStar GXL DNA Polymerase (Takara), or High Fidelity DNA Polymerase (Qiagen). In any of the foregoing embodiments, the DNA polymerase can further include a hot start antibody or aptamer that increases the activation temperature of the DNA polymerase. In such embodiments, the DNA polymerase can be Kapa HiFi Hot Start DNA Polymerase (Roche), NEB Q5 Hot Start DNA Polymerase (NEB), PrimeStar GXL Hot Start DNA Polymerase (Takara), or High Fidelity Hot Start DNA Polymerase (Qiagen).

In any of the foregoing embodiments for splint-mediated primer assembly, the ligation temperature can be about 25° C. to about 40° C. By way of further example, but not limitation, the ligation temperature can be about 25° C. to about 40° C., about 25° C. to about 37° C., about 25° C. to about 35° C., about 25° C. to about 30° C., about 30° C. to about 40° C., about 30° C. to about 35° C., about 35° C. to about 40° C., about 25° C., 26° C., 27° C., 28° C., 29° C., 30° C., 31° C., 32° C., 33° C., 34° C., 35° C., 36° C., 37° C., 38° C., 39° C., or 40° C.

In any of the foregoing embodiments for splint-mediated primer assembly, the ligation duration and be any time sufficient to ligate one of the plurality of first indexing primers to the first 5′ end of the first strand or the second 5′ end of the second strand. In any of the foregoing embodiments, the ligation duration can be from about 5 minutes to about 60 minutes. By way of example, but not limitation, the ligation duration can be about 5 minutes to about 60 minutes, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 60 minutes, about 10 minutes to about 50 minutes, about 10 minutes to about 40 minutes, about 10 minutes to about 30 minutes, about 10 minutes to about 20 minutes, about 20 minutes to about 60 minutes, about 20 minutes to about 50 minutes, about 20 minutes to about 40 minutes, about 20 minutes to about 30 minutes, about 30 minutes to about 60 minutes, about 30 minutes to about 50 minutes, about 30 minutes to about 40 minutes, about 40 minutes to about 60 minutes, about 40 minutes to about 50 minutes, about 50 minutes to about 60 minutes, about 5, 10, 15, 20, 25, 30, 40, 50, or 60 minutes.

In any of the foregoing embodiments for splint-mediated primer assembly, the denaturation temperature can be from about 95° C. to about 98° C. It should be understood that any suitable temperature for denaturing double-stranded DNA to be annealed to in each PCR cycle can be used. It should be understood that the first denaturation temperature should be sufficient that it is higher than a melting temperature of the double-stranded DNA substrate. One of skill in the art can readily determine such temperature and the necessary time through routine experimentation and/or knowledge in the art.

In any of the foregoing embodiments for splint-mediated primer assembly, the annealing temperature can be from about 55° C. to about 65° C. By way of example, but not limitation, the annealing temperature can each be from about 55° C. to about 65° C., 55° C. to about 60° C. , about 60° C. to about 65° C., about 55° C., 56° C., 57° C., 58° C., 59° C., 60° C., 61° C., 62° C., 63° C., 64° C., or 65° C.

In any of the foregoing embodiments for splint-mediated primer assembly, the extension temperature can each be from about 60° C. to about 72° C. By way of example, but not limitation, the extension temperature can be about 60° C., 61° C., 62° C., 63° C., 64° C., 65° C., 66° C., 67° C., 68° C., 69° C., 70° C., 71° C., or 72° C.

In any of the foregoing embodiments for splint-mediated primer assembly, the denaturation duration can be from about 30 seconds to about 2 minutes. By way of example, but not limitation, the denaturation duration can each be from about 30 seconds to about 2 minutes, about 30 seconds to 1.5 minutes, about 30 seconds to 1 minute, about 1 minute to about 2 minutes, about 1 minutes to about 1.5 minutes, about 1.5 minutes to about 2 minutes, about 30 seconds, 45 seconds, 1 minutes, 1.5 minutes, or 2 minutes.

Kits

The present disclosure also provides kits for practicing the methods of the present disclosure. The kits can include any or some of the reagents used in each method and it should be understood that when a reagent is included it can have any of the properties disclosed in the present disclosure and is not limited thereto.

In some embodiments, a kit is provided that includes a first indexing primer having a 3′ terminal portion, a second indexing primer having the same 3′ terminal portion, a first ligase, and a first DNA polymerase. In any of the foregoing kit embodiments, the kit can further include a blocker oligonucleotide comprising a 5′ portion that is at least partially complementary to the 3′ terminal portion. In certain aspects, the blocker oligonucleotide is pre-annealed to the second indexing primer. In any of the foregoing kit embodiments, the blocker oligonucleotide can include a first additional portion 3′ of the 5′ portion which is complementary to the second indexing primer and not complementary to the first indexing primer. In any of the foregoing kit embodiments, the blocker oligonucleotide further can include a 3′ hydroxyl group and a hairpin portion positioned 3′ to the first additional portion, wherein the hairpin portion comprises a first hairpin sequence position 5′ of a second hairpin sequence, wherein the first hairpin sequence and the second hairpin sequence are complementary and can form a hairpin. In such embodiments, the second additional portion can have a length from about 1 to about 30 bases as disclosed in the present disclosure. In such embodiments, the hairpin portion can further include a third hairpin sequence between the first hairpin sequence and the second hairpin sequence. By way of example, but not limitation, the third hairpin sequence can have a length of about 4 bases to about 20 bases. It should be understood that the third hairpin sequence can form a loop sufficient to allow formation of a stable stem-loop structure with the first and second hairpin sequences.

In any of the foregoing kit embodiments, the blocker oligonucleotide can further include a second additional portion positioned between the 5′ portion and the first additional portion, wherein the second additional portion is not complementary to the first indexing primer, and wherein the second additional portion is not complementary to the second indexing primer. In such embodiments, the second additional portion can have a length from about 1 to about 30 bases as disclosed in the present disclosure.

In any of the foregoing kit embodiments, the blocker oligonucleotide further can include a 3′ modification to block polymerase extension if it does not include a hairpin portion. By way of example, but not limitation the 3′ modification to block polymerase extension can be a C3 carbon spacer, hexanediol, spacer 9, spacer 18, phosphate, 2′,3′-dideoxynucleosides ddA, ddT, ddC and ddG, 3′-deoxynucleosides 3′-A, 3′-T, 3′-C and 3′-G, RNA nucleotides such as rU, 3-O-methyl nucleotides, or a DNA sequence that is not complementary to the second indexing primer.

In some embodiments, a kit is provided that can include a first indexing primer having a first 3′ terminal portion, a second indexing primer having a second 3′ terminal portion, a 5′ adapter having a third 3′ terminal portion, a first ligase, and a first DNA polymerase. In such embodiments, the 5′ adapter can further include a first 5′ portion positioned 5′ to the third 3′ terminal potion which can include at least a portion of the first 3′ terminal portion of the first indexing primer, a second 5′ portion positioned 5′ to the first 5′ portion and complementary to the first 5′ portion, and a replication blocker capable of blocking the DNA polymerase positioned at a 5′ end of the first portion. By way of example, but not limitation, the first 5′ portion and the second 5′ portion can each have a length of about 12 bases to about 20 bases as described in the present disclosure. In such embodiments, the 5′ adapter can further include an intervening sequence positioned between the first 5′ portion and the second 5′ portion. By way of example, but not limitation, the intervening sequence can have a length of about 4 to about 20 bases.

In any of the foregoing kit embodiments, the replication block can be selected from the group consisting of a stable abasic site, a C3 spacer, hexandiol, Spacer 9, Spacer 18, 3 or more rU bases, and 2′-O-methyl RNA bases.

In any of the foregoing kit embodiments, the 5′ adapter can have a length from about 25 to about 100 bases as described in the present disclosure.

In any of the foregoing kit embodiments, the first 5′ portion and the second 5′ portion can form a hairpin.

In any of the foregoing kit embodiments, the first indexing primer and the second indexing primer further can include a 5′ tail sequence comprising two or more deoxynucleotides 5′ of three or more ribonucleotide bases, wherein the DNA polymerase has 3′-5′ exonuclease activity. In any of the foregoing kit embodiments, the first indexing primer and the second indexing primer further can further include a 5′ tail sequence comprising the sequence of SEQ ID NO: 1, wherein the DNA polymerase has 3′-5′ exonuclease activity. In such embodiments, the kit can further include a probe oligonucleotide complementary to the 5′ tail sequence and include a modification to provide resistance to digestion by an enzyme with 3′ exonuclease activity, and the enzyme with 3′ exonuclease activity. By way of example, but not limitation, the enzyme with 3′ exonuclease activity can be Exonuclease III. By way of example, but not limitation, the modification to provide resistance to digestion by an enzyme with 3′ exonuclease activity can be at least one phosphorothioate linkage. In such embodiments, the kit can also further include a second ligase. By way of example, but not limitation, the second ligase can be T4 DNA ligase.

In any of the foregoing kit embodiments, the kit can further include a target-specific primer pair. In any of the foregoing kit embodiments, the kit can further include a plurality of target-specific primer pairs. In such embodiments, the kit can further include a universal primer comprising dU bases, ribonucleotides or inosine bases. In such embodiments, the kit can further include an enzyme capable of cleaving the dU bases, ribonucleotides, or inosine bases. Exemplary, non-limiting examples of such enzymes include a combination of uracil DNA glycosylase and Endonuclease VIII, RNase H, and Endonuclease V respectively. In such embodiments, the kit can further include a second DNA polymerase for the multiplex PCR using the target-specific primer pair(s) and universal primer.

In any of the foregoing kit embodiments, the first DNA polymerase and second DNA polymerase can be selected from the group consisting of Kapa HiFi DNA Polymerase (Roche), NEB Q5 DNA Polymerase (NEB), PrimeStar GXL DNA Polymerase (Takara) and High Fidelity DNA Polymerase (Qiagen). By way of further example, but not limitation, the second DNA polymerase can be a uracil tolerant polymerase such as a high fidelity DNA polymerase.

In any of the foregoing kit embodiments, the first DNA polymerase can further include a hot start antibody or aptamer, and wherein the hot start antibody or aptamer increases an activation temperature of the DNA polymerase. In such embodiments, the DNA polymerase can be Kapa HiFi Hot Start DNA Polymerase (Roche), NEB Q5 Hot Start DNA Polymerase (NEB), PrimeStar GXL Hot Start DNA Polymerase (Takara), or High Fidelity Hot Start DNA Polymerase (Qiagen).

In any of the foregoing kit embodiments, the first indexing primer and the second indexing primer can have a length from about 20 bases to about 100 bases as described in the present disclosure.

In any of the foregoing kit embodiments, the ligase is a thermolabile ligase capable of ligation in a low magnesium buffer such as T3 DNA ligase.

It should be understood that throughout the present disclosure, reference to conditions sufficient to denature double-stranded DNA or denature any double-stranded DNA can include conditions where all double-stranded DNA is denatured or where only that which is to be annealed is denatured.

It should be understood also that throughout the present disclosure, reference is made to the synthesis of strands during ligation and PCR cycles and that the use of the term “and” for the production of multiple strands can also be construed as an “or,” at a minimum of on a per molecule basis. It should be further understood that in methods where a partially double-stranded DNA substrate is provided that it can also be a plurality of partially double-stranded DNA substrates and that ligation to or PCR amplification of particular strands can occur on a single molecule or on different substrates and, thus, still fall within the present disclosure. By way of example, but not limitation, the first strand can be ligated on one of the partially double-stranded DNA substrates while the second strand can be ligated on another, in such an instance the third oligonucleotide and fourth oligonucleotide could still be formed.

The following examples are intended to be illustrative of certain embodiments of the present disclosure. The examples should be construed as non-limiting and one of ordinary skill in the art would understand that modifications to the embodiments within the scope of the present disclosure can be made without deviating from the spirit of the disclosure.

### EXAMPLES

### Example 1

### Ligation-Coupled PCR with an Amplicon Panel Targeting the Fungal ITS1 Gene

Rationale

This example illustrates the utility of ligation-coupled PCR as a final indexing and amplification step in preparation of a very small targeted panel for the ITS1 gene. A 15 primer panel was designed to cover the ITS1 gene for multiplexed PCR. The subsequent ligation-coupled-PCR was performed using full-length Illumina TruSeq indexing primers including the common adapter sequence. Blocker oligonucleotide 19-04 was used to prevent ligation of the indexing primer comprising the 3′ adapter (i7), which was pre-annealed to the primer before adding to the ligation-coupled-PCR reaction. The target specific designs for ITS1 originated in Bellemain et al. BMC Microbiol. 2010; 10: 189.

Materials

Candida albicans DNA (ATCC cat# 10231)

Q5 dU bypass Master Mix (2x), (NEB cat# M0598)

15 target-specific primers (Table 1, primers F1-F8 and R9-R15)

Universal primer containing truncated 3′ adapter sequence 14-882 (Table 1)

Indexing primer consisting of full-length 5′ adapter (i5) and enzymatic normalization-compatible 5′ tail (T)₁₂(rU)₄ sequence (SEQ ID NO: 1) 18-460 (Table 1)

Indexing primer consisting of full-length 3′ adapter (i7) with enzymatic normalization-compatible 5′ tail (T)₁₂(rU)₄ (SEQ ID NO: 1) 18-451, pre-annealed to blocker 19-04 (Table 1)

P5 terminal primer for 5′ adapter with enzymatic normalization-compatible tail (T)₁₂(rU)₄ (SEQ ID NO: 1) 18-221 (Table 1)

P7 terminal primer for 3′ adapter with enzymatic normalization-compatible tail (T)₁₂(rU)₄ (SEQ ID NO: 1) 18-222 (Table 1)

SPRIselect reagent (Beckman Coulter cat# B23318)

20% PEG-8000/2.5M NaCl solution

DNA Suspension Buffer, 10 mM Tris, 0.1 mM EDTA, pH 8.0 (Teknova, cat# T0227)

T3 DNA Ligase (Enzymatics, cat# L6010)

USER enzyme (NEB, cat# M5505B)

HiFi PCR Master Mix (Enzymatics, cat# P7670)

Swift Normalase Kit (cat# 66096)

Method

Candida albicans DNA was diluted in DNA suspension buffer. A first reaction for target selection and amplification was performed in 30 ul. For the ITS1 amplicon panel, the forward and reverse target-specific primers comprised a 5′ tail comprising the universal adapter that is a truncated 3′ adapter sequence. This reaction consisted of Q5 dU bypass Master Mix (2×), a mix of 15 target-specific primers F1-F8 and R9-R15 (Table 1) present at equal concentrations of 60 nM each, 10 μM universal primer with modified bases for subsequent cleavage by an endonuclease 14-882 (Table 1) and ing genomic DNA. The following cycling program was run on this reaction mix: 30 seconds at 98° C. followed by 4 cycles of 10 seconds at 98° C., 5 minutes at 63° C., and 1 minute at 65° C., which was then followed by 14 cycles of 10 seconds at 98° C., and 1 minute at 64° C. and a final 1 minute at 65° C. incubation. A purification was performed to remove unused target-specific primers and facilitate a buffer exchange. The purification was performed using 30 ul of SPRIselect beads (1.0x ratio) and the reaction was eluted in 17.4 ul TE but not removed from beads. A second reaction for combined adapter ligation and PCR amplification was performed in 50 ul and included the 17.4 ul eluted reaction mix with beads, lx High-Fidelity PCR Master Mix, 1 uL T3 DNA ligase, 1 uL USER, indexing primers 18-460 and 18-451 at 200 nM concentration for each, and terminal primers 18-221 and 18-222 at 400 nM concentration for each (Table 1). The following cycling program was run for this reaction mix: 20 minutes at 37° C. to allow USER and T3 ligase activity, followed by 30 seconds at 98° C. to inactivate the endonuclease and ligase, denature the DNA substrate and activate the hot start polymerase, and this was followed by 7 cycles of 10 seconds at 98° C., 30 seconds at 60° C., and 1 minute at 66° C. for indexing PCR of the target-specific amplicons. The reaction was then purified with 42.5uL 20% PEG-8000/2.5M NaCl solution (0.85x ratio) and the DNA was eluted in 20 ul DNA Suspension Buffer and transferred to a new tube. The library was quantified using the Kapa qPCR Assay Kit for 11lumina before normalization was completed via the Swift Normalase kit in accordance with the manufacturer's workflow recommendations to achieve a 2 nM pool. The pool was loaded onto an Illumina MiniSeq and sequenced with paired end reads of 151 bases.

Results

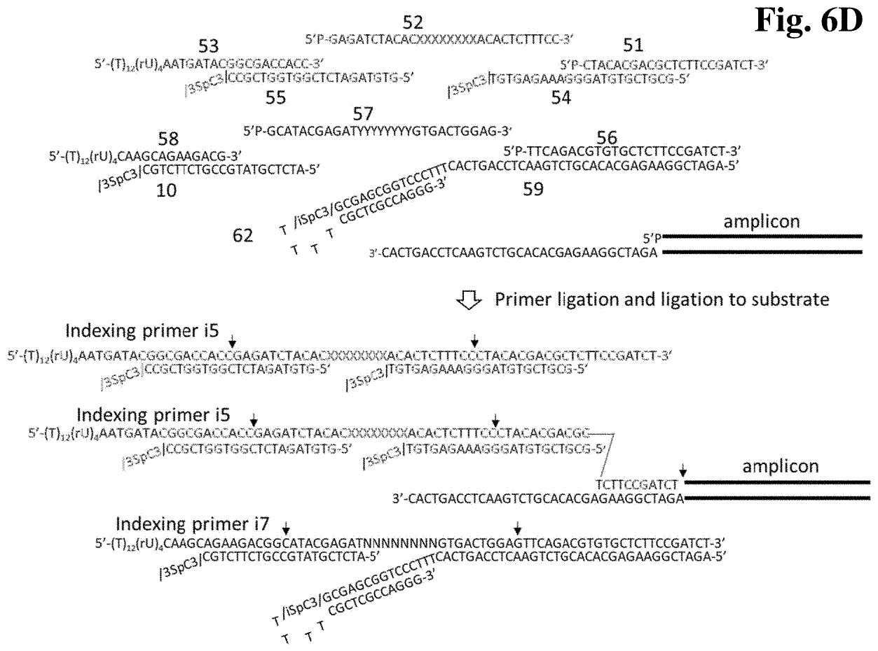

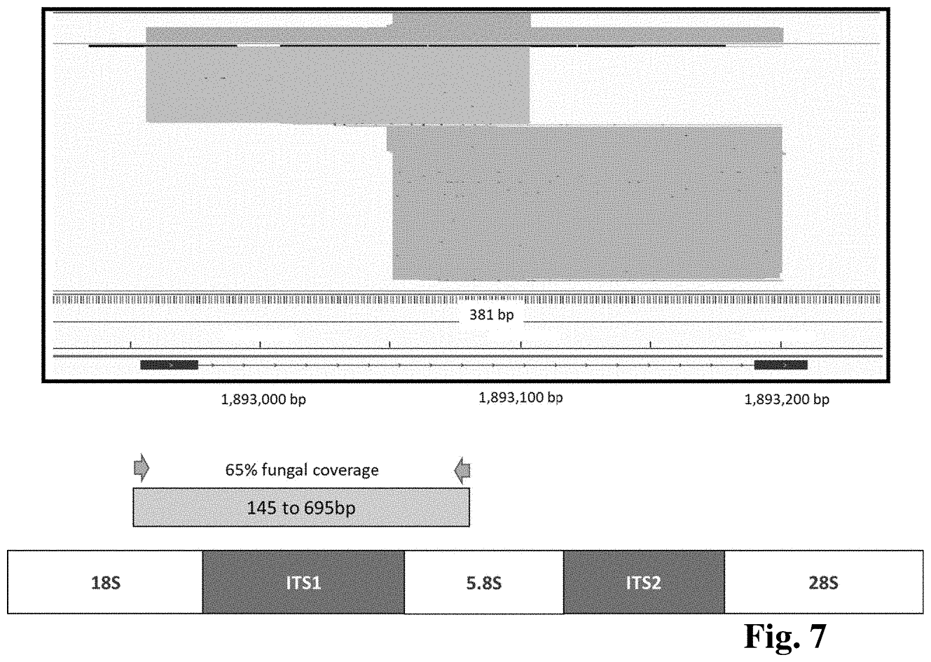

The library yield was 53 nM prior to normalization. The sequencing data was of high quality such that greater than 98% of reads aligned to the intended target region. FIG. 7 depicts the coverage of ITS1 as observed in IGV. Primer dimers are also minimal in the final library such that short reads with an insert size of less than 35 bases represent less than 0.1% of the total reads.

Conclusions

This targeted amplicon library workflow successfully produced sufficient library yields from an extremely small target region of a single locus using 15 primers. The target specific amplicons comprising a first adapter were converted into NGS library and indexed using ligation-coupled-PCR as a second adapter ligation and library indexing strategy. Adapter dimers and primer dimers did not contribute significantly to the final library. The library preparation demonstrated an integrated library preparation and normalization procedure for amplicon-based libraries.

### Example 2

### Ligation-Coupled-PCR for Long Primer Assembly and Enzymatic Library Normalization

Rationale

Ligation-coupled-PCR was used to index, amplify, and condition libraries for enzymatic normalization in a single closed tube. In detail, terminal modified oligos for enzymatic normalization are ligated to indexing primers using a universal splint, followed by PCR cycling to index and amplify libraries with truncated adapters. T3 DNA ligase was used for ligation-based assembly of the primers from oligo subunits in conditions optimized for PCR. The ligation coupled PCR method replaces synthesis of full-length 92 and 86 base oligos that contain both RNA and DNA bases for enzymatic normalization, enabling short universal normalization 5′ tail sequences to be ligated to pre-existing indexing primers for a lower cost of synthesis.

Materials

Enzymatic fragmentation, Swift 2S Turbo Flexible DNA Library Kit (Swift Biosciences, cat# 45024)

T4 DNA Ligase (Rapid) (Enzymatics, cat# L6030-HC-L)

5X Quick Ligation Reaction Buffer (New England Biolabs, cat# 6058)

Truncated adapter; 1st oligonucleotide 13-691 (Table 1)

Truncated adapter; 2nd oligonucleotide 16-349 (Table 1)

Truncated indexing oligos for i5 and i7 18-206, 18-207, 18-210, 18-211, 18-212, 18-213 (Table 1)

Modified Terminal normalization tail oligos 18-204, 18-205 (Table 1)

Universal splints for ligation of truncated indexing oligos to normalization oligos 18-214, 18-215 (Table 1)

T3 DNA Ligase (Enzymatics, cat# L6010L)

PrimeSTAR® GXL SP HiFi DNA Polymerase and Buffer (Takara, cat# RF220Q)

Swift Normalase kit (Swift Biosciences, cat# 66096)

MiSeq Reagent Kit V2, 50cyc1e (I lumina, cat# MS102-2001)

SPRIselect (Beckman coulter, cat# B23419)

Human genomic DNA (Coriell Institute, NA12878)

Methods

Human genomic DNA was re-suspended in a DNA suspension buffer (10 mM Tris-HCl, pH 8.0, 0.1 mM EDTA, pH 8.0). 10 ng of DNA was fragmented, end-repaired, and adenylated using an enzymatic fragmentation mix in a 30 μl reaction. DNA was fragmented to have an average size of 200 bp with 22 min fragmentation time at 32° C., followed by a 30 min inactivation and adenylation at 65° C. Following enzymatic fragmentation, truncated adapters were ligated using a final concentration of 1X Quick Ligation Reaction Buffer, 1200 units T4 DNA ligase, and 1.25 μM of truncated adapters in a 60 μl reaction. The reaction was incubated at 20° C. for 20 minutes. The DNA ligated with truncated adapters was purified using 48 μl SPRIselect beads (ratio: 0.80X) and eluted in 20 μl of DNA resuspension buffer. Stock solutions of 20 μM universal splint oligos were pre-annealed to 27.5 μM modified terminal Normalase protocol oligos by incubating with 50 mM of NaCl at 80° C. for 15 min, and cooling at 20° C. for 30 min. Components for ligation of pre-annealed splint and modified terminal Normalase protocol oligos to modified index primer oligos and amplification of adapted DNA with ligated indexing oligos all took place in a single closed tube reaction; which was assembled in a final volume of 50 μl containing lx HiFi PCR DNA Polymerase and buffer conditions optimized for PCR, 0.6 μM of each i5 and i7 modified indexing oligo, 1 μM of each universal splint oligo, and 1.37 μM of each terminal modified Normalase protocol oligo, 3700 units of T3 DNA Ligase, and 20 μl of purified DNA ligated with truncated adapters. The reaction was placed in a thermocycler with the following program: 25° C. for 15 minutes; 98° C. for 30 sec; 7 cycles of 98° C. for 10 sec, 60° C. for 30 sec, 68° C. for 60 sec; 68° C. for 5 min, and hold at 4° C. The final library was purified using 50 μl of SPRIselect beads (ratio: 1.0X), eluted in 20 μl of TE and assessed by fluorometric methods (Qubit and a high sensitivity DNA Agilent Bioanalyzer kit) and qPCR to ensure the desired library size and confirm the amount. Libraries were normalized to 4 nM using the Swift enzymatic normalization kit and 12 pM was loaded onto a 50 cycle MiSeq cartridge to measure index balance.

Results

The average yield of seven replicate libraries prepared with muliple combinations of indexing primers modified for ligation coupled PCR was 18 nM. The index balance of libraries had a 10.6% coefficient of variation as measured by reads passing filter on the MiSeq, and a cluster density of 752 K/mm².

Conclusions

NGS libraries were successfully indexed, amplified, and conditioned for the enzymatic normalization method using ligation coupled PCR in a single closed tube.

### Example 3

### Efficiency of Primer Assembly Using T3 DNA Ligase in a PCR Mastermix for Ligation-Coupled-PCR

Rationale

This example demonstrates indexing primer assembly by splint ligation in conditions optimized for PCR, which enables combined primer assembly and PCR amplification of substrate molecules in a single closed tube.

Materials

T4 DNA Ligase (Enzymatics cat# L6030-LC-L)

Modified truncated indexing oligos for i5 18-206 (Table 1)

Modified Terminal normalization tail oligo 18-204 (Table 1)

Universal splint oligo for ligation of modified truncated indexing oligos to normalization oligos 18-214 (Table 1)

T3 DNA Ligase (Enzymatics cat# L6010L)

PrimeSTAR® GXL SP HiFi DNA Polymerase and Buffer (Takara cat# RF220Q)

Novex™ TBE-Urea Gels, 15%, 12 well (ThermoFisher Scientific cat# EC68852BOX)

TBE Running Buffer 5× (ThermoFisher Scientific cat# LC6675)

Novex™ TBE-Urea Sample Buffer 2× (ThermoFisher Scientific cat# LC6876)

SYBR Gold Nucleic Acid Gel Stain 10,000× (ThermoFisher Scientific cat# S11494)

TrackIt™ 25 bp DNA ladder (Invitrogen cat# 10488-022)

Methods

A stock solution of 20 μM universal splint oligo (18-214) was pre-annealed to 27.5 μM modified terminal Normalase protocol oligo (18-204) by incubating with 50 mM of NaCl at 80° C. for 15 min, and cooling at 20° C. for 30 min. Components for ligation of pre-annealed splint and modified terminal Normalase protocol oligos to modified index primer oligos took place in a single closed tube reaction; the ligation-coupled-PCR was assembled in a final volume of 50 μl containing lx buffer conditions optimized for PCR, 0.6 μM of i5 modified indexing oligo, 1 μM of universal splint oligo, and 1.37 μM of terminal modified Normalase protocol oligo, 3700 units of T3 DNA Ligase or 300 units of T4 DNA Ligase. The reaction was placed in a thermocycler set at 25° C. for 15 minutes. Some of the reactions were run with 1× HiFi PCR DNA Polymerase added before or after the 15 min incubation. Following incubation, 10 μl of each reaction was mixed with 10 μl of TBE-Urea Sample Buffer, denatured at 95° C. for 2 min, and 18 μl of the denatured mixture was loaded per lane of a TBE Urea gel. The outer most lanes of the gel were loaded with 1.5 μl of a 25 base ladder. Gels were run in a 65° C. oven in 1× TBE Running Buffer for 1 hr at 140 V. The gel was stained using 2× SYBR Gold for 10 min at room temperature, rinsed with distilled water for 1 min, and images were captured with a UV light source.

Results

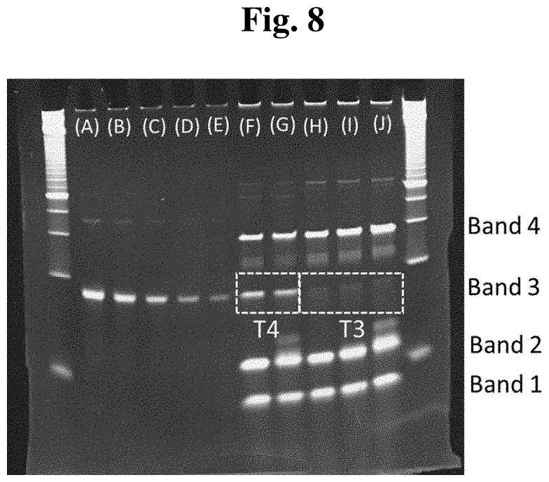

As shown on the gel (FIG. 8), Band 4 shows ligated product (73 bases) and Band 1 is the universal splint oligo (18-214, 24 bases) used to ligate the terminal Normalase oligo (Band 2, 18-204, 28 bases) to the modified indexing primer (Band 3, 18-206, 45 bases). Lanes (A) through (E) show 100%, 60%, 40%, 20%, and 10% of the modified indexing oligo alone, respectively, which is the limiting oligo in the ligation reaction. Lanes (F) and (G) demonstrate only partial ligation of the modified indexing oligo using T4 DNA ligase in conditions optimized for PCR without and with HiFi DNA Polymerase. Lanes (H), (I), and (J) demonstrate over 90% ligation of modified indexing oligo using T3 DNA ligase incubated in reaction conditions optimized for PCR without and with HiFi DNA polymerase added after and before incubation for 15 min at 25° C., respectively.

Conclusion

Results demonstrate over 90% ligation using T3 DNA ligase in conditions optimized for PCR, whereas only partial ligation is observed using T4 DNA ligase.

### Example 4

### Ligation-Couple PCR with Truncated Indexing Primers and Hairpin 5′ Adapter for a DNA NGS Library Method Utilizing Sequential Adapter Attachment Chemistry and a 3′ Adapter with Blunt End

Rationale

This example demonstrates feasibility of the ligation-coupled PCR reaction with truncated indexing primers i5 and i7 lacking 13 bases at the 3′ terminus and a truncated hairpin 5′ adapter. It also demonstrates the advantage of the hairpin 5′ adapter over the linear 5′ adapter (as shown on FIGS. 3A and 3B). Due to 13 base 3′ end truncation, the truncated indexing primer i5 (domain 3 in FIGS. 3A, B) can't be used as a 5′ adapter so additional 5′ adapter must be added to the ligation-coupled PCR reaction. The 3′ adapter utilized in this example is a blunt-ended truncated adapter (domain 2); whereas, the 5′ adapter is either a truncated hairpin (domain 5 in FIG. 3B) with a relatively short single stranded 3′ portion or a truncated linear adapter (domain 5 in FIG. 3A) with substantially higher Tₘ. Melting temperature of the stem region of the hairpin 5′ adapter used in this example is very high (Tₘ˜80° C.) so that during primer annealing and extension the hairpin 5′ adapter is folded and does not participate in PCR due to low Tₘ of its single stranded 3′ portion (Tₘ ˜50° C.). Melting temperature of the linear truncated 5′ adapter is substantially higher (Tₘ ˜73° C.) making highly possible involvement of this adapter in PCR amplification and production of library molecules not capable to support cluster formation on a flow cell during sequencing. In addition, the hairpin adapter region contains unreplicable spacer to prevent hairpin replication and creation the replication of undesirable products during ligation-coupled PCR reaction.

In this example, the deoxyuridine containing strand represented by oligonucleotide 1 was fully degraded by UDG enzyme, followed by annealing of the 5′ truncated hairpin adapter to the remaining single strand of 3′ adapter (oligonucleotide 1) and the ligation to the 5′ end of the DNA substrate via T3 DNA ligase. Further, the inactivation of UDG and T3 DNA ligase at 95-98° C. and activation of a hot-start thermostable DNA polymerase simultaneously occurred, followed by a subsequent PCR of NGS DNA libraries in the presence of truncated indexing primers, all in a single tube incubation.

Materials

T4 DNA Ligase (Rapid) (Enzymatics cat# L6030-HC-L)

Oligonucleotide sequences (Table 1)

5X Quick Ligation Reaction Buffer (New England Biolabs, cat# 6058)

T3 DNA Ligase (Enzymatics cat# L6010L)

Uracil-DNA Glycosylase (Enzymatics cat# G5010L)

2X HiFi PCR MasterMix (Enzymatics, cat# P7670)

100 mM 2′-deoxynucleoside 5′-triphosphate (dNTP) Set (Invitrogen, cat# 10297018)

T4 DNA polymerase (Enzymatics, cat# P7080L)

T4 Polynucleotide Kinase (Enzymatics, cat# Y904L)

SPRIselect (Beckman coulter, cat# B23419)

human genomic DNA (Coriell Institute, NA12878)

DNA Suspension Buffer, 10 mM Tris, 0.1 mM EDTA, pH 8.0 (Teknova, cat# T0227

Methods

Human genomic DNA was re-suspended in a DNA suspension buffer (10 mM Tris-HCl, pH 8.0, 0.1 mM EDTA, pH 8.0) at a concentration of 100 ng/μl. The DNA was fragmented with a Covaris M220 Focused-Ultrasonicator to 200 base pairs average size. A tight distribution of fragmented DNA from about.150 bp to about.250 bp was obtained.

The fragmented DNA was used to prepare NGS libraries. The polishing reaction was assembled in 30 μl comprising 100 μM of each dNTP, 0.15 units T4 DNA polymerase, 10 units of T4 Polynucleotide Kinase. The reaction was incubated at 20° C. for 20 minutes, followed by heat inactivation at 65° C. for 10 minutes. The 3′ adapter ligation reaction was assembled in 60 ill including, 1X Quick Ligation Reaction Buffer, 220 pmoles of the 3′ adapter t oligonucleotide 13-712 110 pmoles of the 3′ adapter oligonucleotide 13-690, the 30 μl of polished DNA and 1200 units of T4 DNA ligase. The ligation reaction was incubated at 20° C. for 20 minutes. The DNA was purified using 48 μl SPRlselect beads (ratio: 0.8X). DNA was eluted in 18.5 μl of DNA suspension buffer. The ligation-coupled, indexing PCR reaction was assembled in 50 μl volume containing 1X HiFi PCR MasterMix, 1 pmole of truncated hairpin 5′ adapter oligonucleotide 19-351 or linear 5′ adapter 19-31, 60 pmoles of indexing primer i5 oligonucleotide 17-277, 60 pmoles of indexing primer oligonucleotide 14-161, 1500 units of T3 DNA Ligase, 2 units of uracil-DNA glycosylase, and 18 μl of DNA purified after the 3′ adapter ligation reaction. The reaction was placed in a thermocycler with the following set program: 37 ° C. for 20 minutes, followed by 98° C. for 2 minutes, followed by 7 cycles of (98° C. for 20 seconds, 60° C. for 30 seconds, 72° C. for 60 seconds), followed by 72° C. for 1 min, and Hold at 4° C. The final library was purified using 42.5 μl of SPRlselect beads (ratio: 0.8X). The DNA was eluted in 20 μl of DNA suspension buffer and assessed by fluorometric methods (Qubit and a high sensitivity DNA Agilent Bioanalyzer kit) and qPCR to ensure desired library size and confirm quantification.

The truncated, blunt-ended 3′ adapter was prepared by annealing of two oligonucleotides (as described in U.S. Pat. No 10,208,338, incorporated herein by reference in its entirety): oligonucleotide 13-690 with a phosphate group at the 5′ end and a blocking group (such as a C3 spacer) at the 3′ end, and oligonucleotide 13-712 comprising degradable deoxyuridine bases and an un-ligatable 3′-dT base at the 3′ end (the thymine base modification lacking a hydroxyl group at the 3′ ribose position).

Results

Table 2 outlines experimental results obtained by quantitative assessment of library yield using Qubit and the Agilent Bioanalyzer. The 5′ truncated hairpin adapter maintained the efficiency of ligation-coupled indexing PCR reaction as demonstrated by equivalent library yields compared to the condition in which full size indexing primers were used and the i7 adapter was added after ligation of the adapter i5 was completed. Furthermore, replacing the 5′ truncated hairpin adapter with a linear adapter resulted in a reduction of library yield.

Conclusion

NGS libraries were successfully made using a 3-step protocol with sequential ligation of two NGS adapters where the first step repaired DNA ends, the second step added a 3′ blunt end adapter, and the final step ligated a 5′ hairpin adapter, added indexes and amplified NGS library by PCR in a single, closed tube reaction using truncated indexing primers, as shown in FIG. 1G and FIG. 3B. Use of truncated hairpin 5′ adapter consisting of an un-replicable spacer within the loop region improves library yield and prevents formation of non-desirable products during ligation-coupled PCR reaction.

### Example 5

### Ligation-Couple PCR with Full Size Indexing Primers and Primer Blocker for a DNA NGS Library Method Utilizing Sequential Adapter Attachment Chemistry and a 3′ Adapter with Blunt End

Rationale

This example demonstrates feasibility of the ligation-coupled PCR reaction with full size indexing primers, where one of indexing primers (i5) is used as a 5′ adapter and become ligated to DNA and, where ligation of the second indexing primer (i7) is prevented by blocker oligonucleotide pre-annealed to the second indexing primer as shown in FIG. 1E and FIG. 2B. The example shows the utility of a blocker oligonucleotides containing cleavable dU bases and mismatches (FIG. 2F and FIG. 2G) by demonstrating a substantially lower library yield in the absence of blocker oligonucleotide (FIG. 2A). It also shows that both the number and the position of mismatches and cleavable bases within the blocker oligonucleotide are important for its performance. Blocker oligonucleotide inactivation during PCR reaction is achieved by oligonucleotide fragmentation at high temperature at the abasic sites generated during combined 5′ adapter ligation by T3 DNA ligase and incubation with uracil deoxy glycosylase at 37° C. as is shown in FIG. 2F and FIG. 2G. The last reaction helps to dissociate 3′ adapter oligonucleotide with dU bases from the oligonucleotide attached to DNA but does not disrupt substantially the interaction between blocker oligonucleotide and indexing primer i7. The 3′ adapter utilized in this example is a blunt-ended truncated adapter. All three reaction, including UDG treatment, 5′ adapter ligation by T3 DNA ligase and library amplification in the presence of full size indexing primers occurs in a single, closed tube reaction.

Materials

T4 DNA Ligase (Rapid) (Enzymatics cat# L6030-HC-L)

Oligonucleotide sequences (Table 1)

5X Quick Ligation Reaction Buffer (New England Biolabs, cat# 6058)

T3 DNA Ligase (Enzymatics cat# L6010L)

Uracil-DNA Glycosylase (Enzymatics cat# G5010L)

2X HiFi PCR MasterMix (Enzymatics, cat# P7670)

100 mM 2′-deoxynucleoside 5′-triphosphate (dNTP) Set (Invitrogen, cat# 10297018)

T4 DNA polymerase (Enzymatics, cat# P7080L)

T4 Polynucleotide Kinase (Enzymatics, cat# Y904L)

SPRIselect (Beckman coulter, cat# B23419)

human genomic DNA (Coriell Institute, NA12878)

DNA Suspension Buffer, 10 mM Tris, 0.1 mM EDTA, pH 8.0 (Teknova, cat# T0227

Methods

Human genomic DNA was re-suspended in a DNA suspension buffer (10 mM Tris-HCl, pH 8.0, 0.1 mM EDTA, pH 8.0) at a concentration of 100 ng/μl. The DNA was fragmented with a Covaris M220 Focused-Ultrasonicator to 200 base pairs average size. A tight distribution of fragmented DNA from about.150 bp to about.250 bp was obtained.

The fragmented DNA was used to prepare NGS libraries. The polishing reaction was assembled in 30 μl comprising 100 μM of each dNTP, 0.15 units T4 DNA polymerase, 10 units of T4 Polynucleotide Kinase. The reaction was incubated at 20° C. for 20 minutes, followed by incubation at 65° C. for 10 minutes. The 3′ adapter ligation reaction was assembled in 60 μl including, 1X Quick Ligation Reaction Buffer, 220 pmoles of the 3′ adapter oligonucleotide 13-712, 110 pmoles of the 3′ adapter oligonucleotide 13-690, the 30 μl of polished DNA and 1200 units of T4 DNA ligase. The reaction was incubated at 20° C. for 20 minutes. The DNA was purified using 48 μl SPRlselect beads (ratio: 0.8X). DNA was eluted in 18.5 μl of DNA resuspension buffer. The ligation-coupled, indexing PCR was assembled in 50 μl reaction volume containing 1X HiFi PCR MasterMix, 60 pmoles of full size indexing primer i5 oligonucleotide 19-34, 60 pmoles of full size indexing primer i7 oligonucleotide 19-37, 72 pmoles of indexing primer i7 blocker, 1500 units of T3 DNA Ligase, 2 units of uracil-DNA glycosylase, and 18 μl of the DNA purified after the 3′ adapter ligation reaction. The reaction was placed in a thermocycler with the following set program: 37° C. for 20 minutes, followed by 98° C. for 2 minutes, followed by 7 cycles of 98° C. for 20 seconds, 60° C. for 30 seconds, 72° C. for 60 seconds), followed by 72° C. for 1 min, and Hold at 4° C. The final library was purified using 42.5 μl of SPRIselect beads (ratio: 0.85X). The DNA was eluted in 20 μl and assessed by fluorometric methods (Qubit and a high sensitivity DNA Agilent Bioanalyzer kit) and qPCR to ensure desired library size and confirm quantification.

The truncated, blunt-ended 3′ adapter was prepared by annealing of two oligonucleotides (as described in U.S. Pat. No. 10,208,338, incorporated herein by reference in its entirety): oligonucleotide 13-690 with a phosphate group at the 5′ end and a blocking group (such as a C3 spacer) at the 3′ end, and oligonucleotide 13-712 comprising degradable deoxyuridine bases and an un-ligatable 3′-dT base at the 3′ end (the thymine base modification lacking a hydroxyl group at the 3′ ribose position) base at the 3′ end (the thymine base modification lacking a hydroxyl group at the 3′ ribose position).

Results

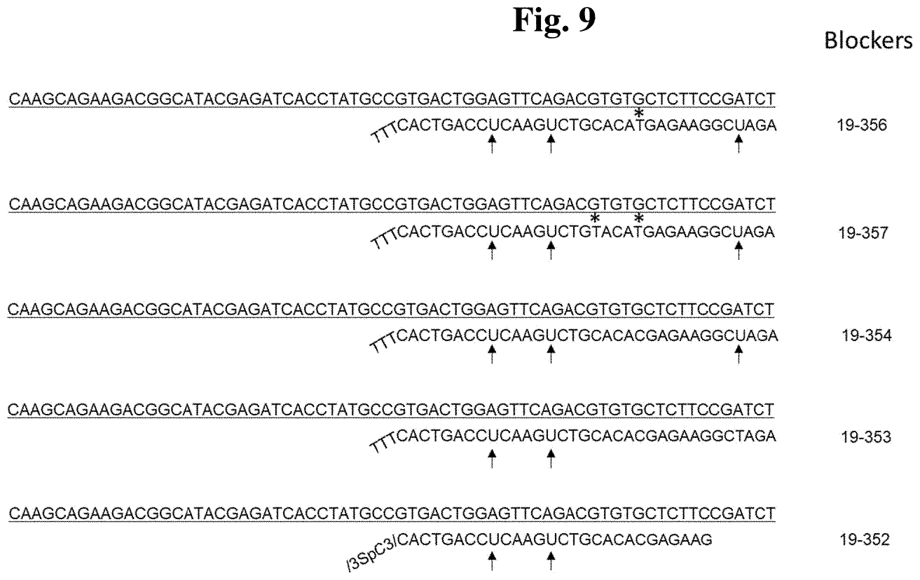

The sequences of all tested primer blockers listed in Table 1 as oligonucleotides 19-356, 19-357, 19-352, 19-353 and 19-354) are shown in FIG. 9 in complex with their target, indexing primer P7 oligonucleotide 19-37. Table 2 outlines experimental results obtained by quantitative assessment of library yield using Qubit and the Agilent Bioanalyzer. The efficiency of ligation-coupled indexing PCR reaction was highly dependent on the addition of the Blocker oligonucleotide and its composition. All blocking oligonucleotides shown in FIG. 9 efficiently blocked primer i7 and blocker oligonucleotide 19-356 containing 3 dU bases and one T*G mismatch resulted in the maximum increase in the library yield, comparable with the condition in which the indexing primers were added after the ligation of the i5 primer-adapter was completed. There was a 4-fold reduction in the library yield when no blocker was added to the ligation-couple PCR, but to a less extent when other blockers were used.

Conclusion

NGS libraries were successfully made using a 3-step protocol with sequential ligation of two NGS adapters where the first step repaired DNA ends, the second step added a 3′ blunt end adapter, and the final step ligated a 5′ primer-adapter i5, added indexes and amplified NGS library by PCR in a single, closed tube reaction using full size indexing primers, as shown in FIG. 1E and FIG. 2B. Use of primer blocker containing dU bases and a mismatch improves library yield 4-fold and allows to achieve the same performance as in the control case characterized by 100% incorporation of the 5′ adapter.

### Example 6

### Ligation-Coupled PCR with Truncated Indexing Primers and Hairpin 5′ Adapter for a DNA NGS Library Method Utilizing Sequential Adapter Attachment Chemistry and 3′ Adapter with Single Base Overhang

Rationale

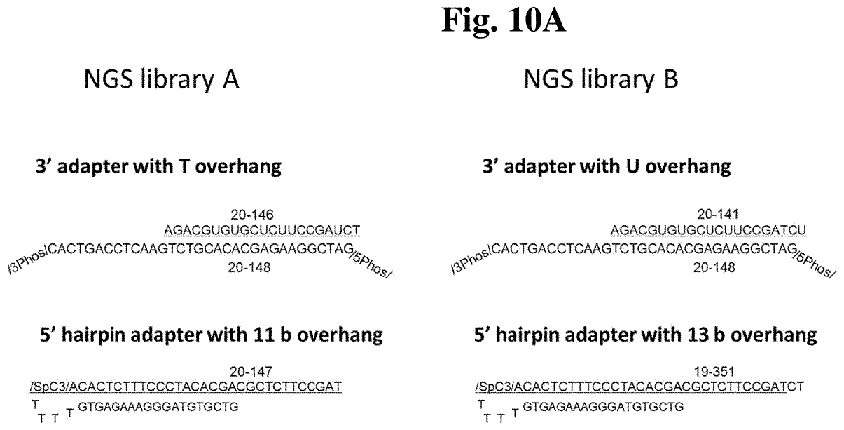

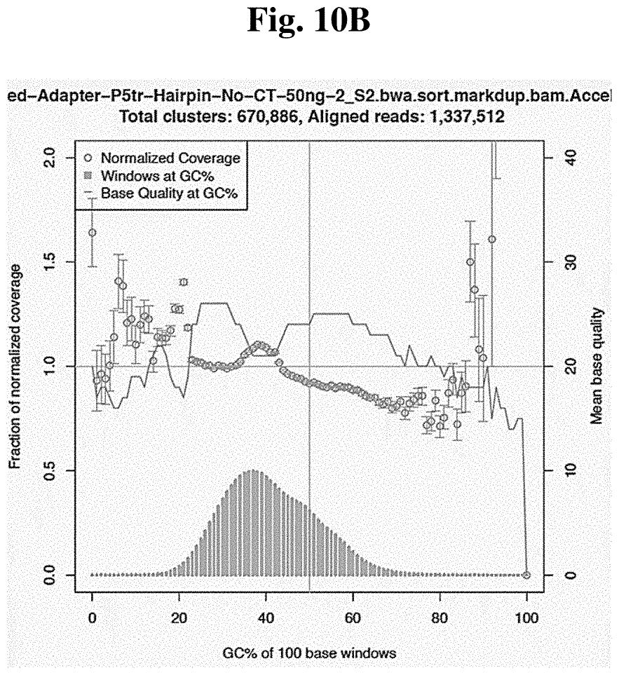

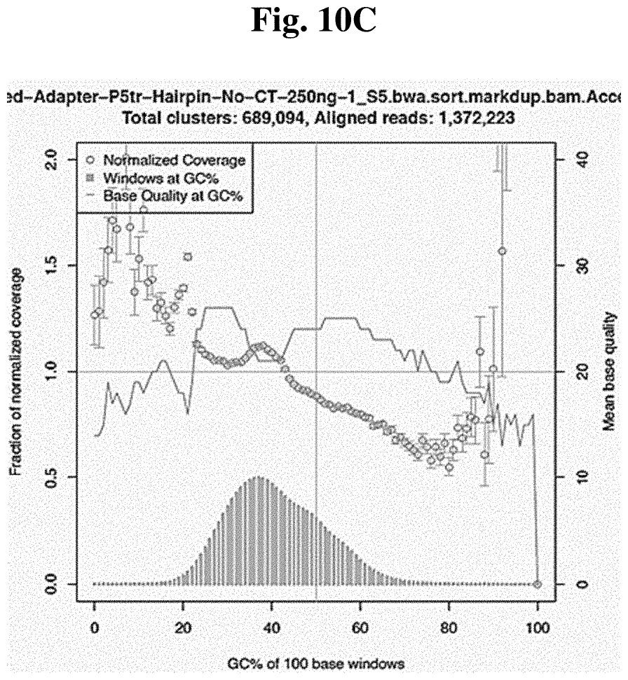

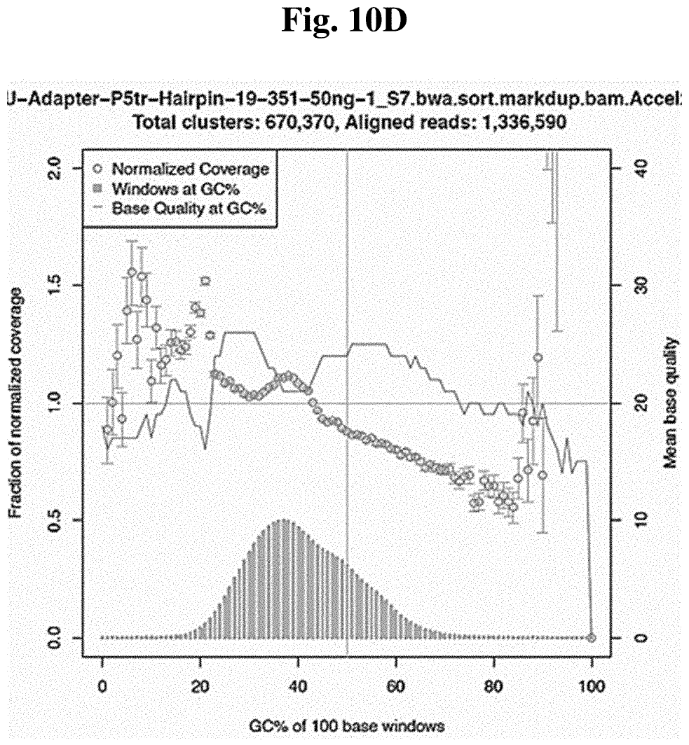

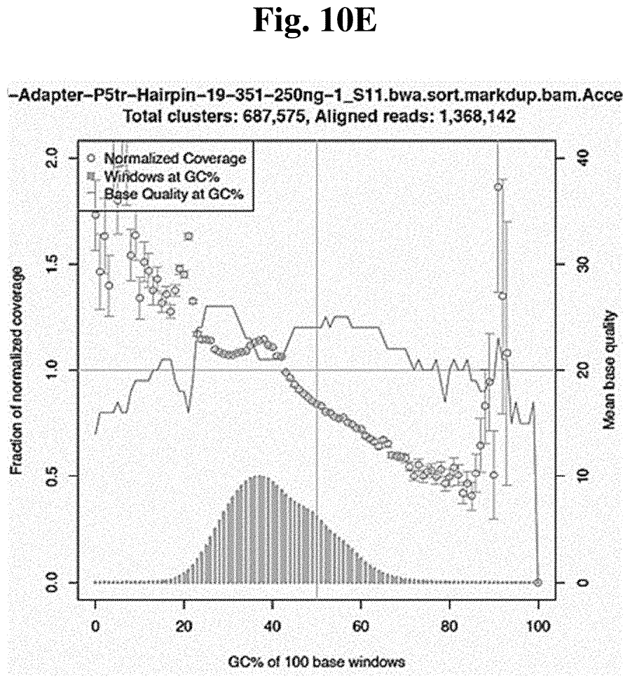

This example demonstrates feasibility of the ligation-coupled PCR reaction utilizing truncated indexing primers i5 and i7 lacking 13 bases at the 3′ terminus and truncated hairpin 5′ adapter. The 3′ adapter used in this example is a truncated adapter with either T or U single base 3′ overhang (FIGS. 3K and 31, respectively), whereas the 5′ adapter is a truncated hairpin adapter (domain 5 in FIGS. 3K and 3I) with 11 b or 13 b single stranded 3′ portion, respectively. Melting temperature of the stem region of the hairpin 5′ adapter used in this example is 80° C. suggesting that during primer annealing and extension PCR phase the hairpin adapter is folded and not participate in PCR due to low Tₘ of its remaining single stranded 3′ portion (Tm ˜50° C.). In addition, the hairpin loop region has an unreplicable spacer to prevent its replication and creation of other undesirable products during ligation-coupled PCR reaction. FIG. 10A shows the 3′ and 5′ adapters used in this example.

In this example, the deoxyuridine containing 3′ adapter strand represented by oligonucleotide 1 was fully degraded by USER enzyme, followed by annealing of the 5′ truncated hairpin adapter to the remaining single strand of the 3′ adapter (oligonucleotide 2) and ligation via T3 DNA ligase. In the case of the 3′ adapter with a single base U overhang ligation occurred between the 13 b single stranded portion of the hairpin adapter and the 5′ end of DNA fragment (FIG. 31), while in the case of the 3′ adapter with a single base T overhang ligation occurred between the 11 b single stranded portion of the hairpin adapter and the CT dinucleotide portion of the oligonucleotide 1 covalently attached to DNA fragment (FIG. 3K). Use of the 3′ adapter with non-ligatable ddT or 3′-dT base analog in this case is not as useful, as in the case of the blunt end 3′ adapter (Examples 4 and 5), due to low efficiency of ligation of the adapter with single base overhang to only one DNA strand (data nor shown). Further, the inactivation of USER and T3 DNA ligase at 95-98° C. and activation of a hot -start thermostable DNA polymerase simultaneously occurred, followed by a subsequent PCR of NGS DNA libraries in the presence of truncated indexing primers, all in a single tube incubation.

The example demonstrates ability of the described approach to produce NGS libraries using universal concentrations of the 3′ and 5′ adapters in a very broad range of DNA input, and the lack of adapter dimer formation at DNA inputs down to femtogram level.

Materials

T4 DNA Ligase (Rapid) (Enzymatics cat# L6030-HC-L)

Oligonucleotide sequences (Table 1)

5X Quick Ligation Reaction Buffer (New England Biolabs, cat# 6058)

T3 DNA Ligase (Enzymatics cat# L6010L)

USER (New England Biolabs cat#M5505L)

2X HiFi PCR MasterMix (Enzymatics, cat# P7670)

100 mM 2′-deoxynucleoside 5′-triphosphate (dNTP) Set (Invitrogen, cat# 10297018)

T4 DNA polymerase (Enzymatics, cat# P7080L)

T4 Polynucleotide Kinase (New England Biolabs, cat# M0202L)

Taq DNA Polymerase (Enzymatics, cat# P72250L)

SPRIselect (Beckman coulter, cat# B23419)

human genomic DNA (Coriell Institute, NA12878)

DNA Suspension Buffer, 10 mM Tris, 0.1 mM EDTA, pH 8.0 (Teknova, cat# T0227

Methods

Human genomic DNA was re-suspended in a DNA suspension buffer (10 mM Tris-HCl, pH 8.0, 0.1 mM EDTA, pH 8.0) at a concentration of 100 ng/μl. The DNA was fragmented with a Covaris M220 Focused-Ultrasonicator to 200 base pairs average size. A tight distribution of fragmented DNA from about.150 bp to about.250 bp was obtained.

The fragmented DNA was used to prepare NGS libraries. The amount of DNA used for library preparation varied from 250 ng down to 0.3 fg. In some experiments, with very low DNA inputs, 3.3pg of single stranded DNA was added as a carrier to prevent loss of DNA during library preparation. The polishing reaction was assembled in 30 μl comprising 100 μM of each dNTP, 0.15 units T4 DNA polymerase, 10 units of T4 Polynucleotide Kinase, and 1.5 units of Taq DNA Polymerase. The reaction was incubated at 20° C. for 20 minutes, followed by incubation at 65° C. for 10 minutes. The 3′ adapter ligation reaction was assembled in 60 μl including, 1X Quick Ligation Reaction Buffer, 220 pmoles of the 3′ adapter oligonucleotide 20-141 or 20-146, 110 pmoles of the 3′ adapter oligonucleotide 20-148, 30 μl of polished DNA and 1200 units of T4 DNA ligase. The reaction was incubated at 20° C. for 20 minutes. The DNA was purified using 48 μl SPRIselect beads (ratio: 0.8X). DNA was eluted in 18.5 μl of DNA resuspension buffer. Degradation of the dU-containing strand of 3′ adapter, annealing and ligation of the truncated hairpin 5′ adapter oligonucleotide, and amplification of the adapted DNA all took place in asingle, closed-tube reaction which was assembled in a final volume of 50 ill containing 1X HiFi PCR MasterMix, 1 pmole of truncated hairpin 5′ adapter oligonucleotide 20-147 or 19-351, 60 pmoles of the truncated indexing primer i5 oligonucleotide 17-277, 60 pmoles of the truncated indexing primer i7 oligonucleotide 14-161, 1500 units of T3 DNA Ligase, 1 unit of USER, and 19 μl of the DNA purified after the 3′ adapter ligation reaction. The reaction was placed in a thermocycler with the following set program: 37° C. for 20 minutes, followed by 98° C. for 2 minutes, followed by 7 cycles of (98° C. for 20 seconds, 60° C. for 30 seconds, 72° C. for 60 seconds), followed by 72° C. for 1 min, and Hold at 4° C. The final library was purified using 42.5 μl of SPRIselect beads (ratio: 0.85X). The DNA was eluted in 20 μl of DNA suspension buffer and assessed by fluorometric methods (a high sensitivity DNA Agilent Bioanalyzer kit) and qPCR to ensure desired library size and confirm quantification. Quantified libraries were pooled, loaded onto an Illumina MiniSeq and sequenced with paired end reads of 151 bases.

Results

Table 4 outline experimental results obtained by quantitative assessment of the library yield using the Agilent Bioanalyzer and qPCR. Table 5 and FIGS. 10A-10E outlines sequence metrics obtained for two separate library preparation conditions as outlined in Rationale.

NGS libraries synthesized using sequential adapter ligation chemistry with the 3′ adapter containing single base overhang, and ligation-coupled indexing PCR produced equally good results independent of what type of terminal 3′ base was utilized in the 3′ adapter, T or U. The library protocol utilizing 3′ adapter with T overhang and hairpin 5′ adapter with 11 b overhang (NGS Library A in FIG. 10A) was a little bit more efficient for lower input samples (50 and 100 ng) while the protocol utilizing 3′ adapter with U overhang and hairpin 5′ adapter with 13 b overhang (NGS Library B in FIG. 10A) was more efficient for higher input samples (250 ng) (Table 4). Sequencing metrics for both methods are pretty good and very similar (Table 5). Picard plots presented on FIGS. 10B-10C (NGS library A) demonstrate AT/GC bias typical for NGS libraries prepared by T/A ligation chemistry with Y adapters (data not shown). NGS library B demonstrates stronger AT/GC bias, especially for highest DNA input (250ng) (FIGS. 10D-10E). Such bias is most likely a consequence of the reduced cleavage efficiency of the dU base at the junction with GC-rich DNA sequences by USER enzyme during ligation-coupled PCR reaction. The cleavage improves and the bias become lower when the dU base is positioned within the 3′ adapter sequence as in the case of NGS library A where the 3′ adapter has T base at the 3′ end.

When NGS library method A was applied to very low DNA inputs it demonstrated lack of adapter-dimers down to sub-femtogram amount. Table 6 and FIGS. 11A-11B show Agilent Bioanalyzer traces of NGS libraries prepared by method A in 3 pg-100 pg DNA input range for DNA, while Table 7 and FIG. 11C illustrate libraries prepared for extremely low DNA samples in the 0.3 fg — 3pg range. Despite the such low amount of DNA and the large number of PCR cycles (30) no adapter-dimers were observed. It is important to emphasize that all library traces shown in FIGS. 11A-11C were generated by NGS libraries prepared with the same adapter concentration.

Conclusion

NGS libraries with adapter ligation chemistry that involves sequential addition of the 3′ and 5′ adapters, where attachment of the 5′ adapter and library amplification occur in a single, closed-tube ligation-coupled PCR reaction, and where the 3′ adapter has T or U base 3′ overhang can be efficiently used for high and low DNA samples using one universal protocol without any adapter concentration adjustment. The NGS library can be prepared from samples containing traces amount of DNA and potentially useful for forensic applications, ancient DNA samples and single cell sequencing of bacterial DNA.

### Example 7

### Ligation-Couple PCR with Full Size Indexing Primers and Primer Blockers for 16S DNA Amplicon Panel

Rationale

This example demonstrates utility of the primer blocker annealed to the P7 indexing primer in the amplicon workflow with ligation-coupled PCR step. Amplicon panel containing 23 16S primers was used to test various i7 primer blockers. The target specific primers included a truncated adapter P7 sequence used during multiplex PCR as a universal primer sequence and during ligation-coupled PCR as a degradable adapter region for incorporation of the P5 adapter sequence.

Materials

E. coli DNA (ATCC, cat# EDL933)

Q5® dU ByPass High-Fidelity 2X Master Mix (NEB, cat# M0494)

23 target-specific primers P1-P23 (Table 1)

Universal primer containing truncated P7 adapter sequence 14-882 (Table 1)

Indexing primers consisting of full-length P7 adapter sequence 18-453 (Table 1)

Indexing primer consisting of full-length P5 adapter sequence 18-452 (Table 1)

Blocking oligo corresponding in part to the 3′ terminal end of the P7 adapter 19-04 (Table 1)

Primers corresponding to the terminal regions of the adapter sequences (Table 1)

SPRIselect reagent (Beckman Coulter, B23318)

20% PEG-8000/2.5M NaC1 solution

DNA Suspension Buffer, 10 mM Tris, 0.1 mM EDTA, pH 8.0 (Teknova, cat# T0227)

T3 Ligase (Enzymatics, cat# L6010)

USER enzyme (NEB, cat# M5505B)

HiFi PCR Master Mix (Enzymatics, cat# P7670)

Qubit™ dsDNA HS Assay Kit (Thermo Fisher Scientific, Q32851)

Methods

E. coli DNA was diluted in DNA suspension buffer. A first reaction for target selection was performed in 30 ul. For the target amplicons the forward target-specific primer and the reverse target-specific primer contained a truncated P7 adapter sequence at the 5′ end. This reaction consisted of 1× Q5 dU High-Fidelity Master Mix, a mix of 23 target-specific primers P1-P23 present at variable concentrations, between 30-90nM (Table 1), 10 uM of oligo 14-882, and 1 ng E. coli DNA. The following cycling program was run on this reaction mix: 30 seconds at 98° C. followed by 4 cycles of 10 seconds at 98° C., 5 minutes at 63° C., and 1 minute at 65° C., which is then followed by 14 cycles of 10 seconds at 98° C., and 1 minute at 64° C. A final finishing step of 1 minute at 65° C. generates target-specific amplicons. A purification was performed to maximize removal of target-specific primers and facilitate changes in buffer. The purification was done with 30 ul of SPRIselect beads (1.0× ratio) and the reaction was eluted in 17.4 ul TE, but not removed from beads. A second reaction for combined adapter ligation of target-specific amplicons and PCR amplification. This reaction contained 17.4 ul eluted reaction mix with beads, 1× HiFi PCR Master Mix, 400 nM primers corresponding to the terminal sequences of the adapters (sequences 18-204 and 18-205), T3 DNA ligase, USER, and indexing primers (sequences 18-452 and 18-453). Libraries were prepared both with and without 1 uM of a P7 blocking oligo (sequence 19-04) in the second PCR reaction. The following cycling program was run on this reaction mix: 20 minutes at 37° C., followed by 30 seconds at 98° C., and followed by 8 cycles of 10 seconds at 98° C., 30 seconds at 60° C., and 1 minute at 66° C. for amplification of target-specific amplicons. The reaction was then purified with 42.5uL 20% PEG-8000/2.5M NaCl solution (0.85x ratio) and the DNA was eluted in 20 ul DNA Suspension Buffer. Libraries were quantified using a Qubit^{Tm} dsDNA HS kit (Thermo Fisher Scientific).

Results

Libraries gave consistent and expect yields, with an average yield (n=2) of 17.6 ng/uL for libraries without a blocker oligo and an average yield (n=2) of 25.5 ng/uL for libraries containing a blocker oligo. The difference in average yield was approximately 44.9%.

Conclusions

A targeted 16S amplicon library was successfully made using a truncated P7 sequence introduced through the target specific primers. The target specific amplicons were indexed using a one-step, closed-tube ligation-amplification strategy. The 1.5-fold difference in yield between amplicon libraries with and without a blocking oligo is substantially lower than in the case of NGS DNA library (Example 4) where the difference is 4-fold. This example demonstrates that amplicon libraries can be efficiently created and amplified without a blocking oligonucleotide in the ligation-coupled PCR reaction.

TABLE 1
Oligonucleotide sequences (extended table)

| Identifier | Sequence | SEQ ID NO |
|---|---|---|
| 14-882 | TCAGACGUGUGCUCUUCCGAU*C*U | 3 |
| 18-460 | TTTTTTTTTTTTrUrUrUrUAATGATACGGCGACCACCG | 4 |
| | AGATCTACACGTACTGACACACTCTTTCCCTACACG | |
| | ACGCTCTTCCGATCT | |
| 18-451 | TTTTTTTTTTTTrUrUrUrUCAAGCAGAAGACGGCATAC | 5 |
| | GAGATGCGCGAGAGTGACTGGAGTTCAGACGTGTGC | |
| | TCTTCCGATCT | |
| 18-221 | TTTTTTTTTTTTrUrUrUrUAATGATACGGCGACCACCG | 6 |
| | AGA | |
| 18-222 | TTTTTTTTTTTTrUrUrUrUCAAGCAGAAGACGGCATAC | 7 |
| | GAGAT | |
| 19-04 | AGATCGGAAGAGCTTTTTTACACGTCTGAACTCC*T* | 8 |
| | T/3SpC3/ | |
| F1 | TCAGACGTGTGCTCTTCCGATCTCTTGGTCATTTAGA | 9 |
| | GGAAGTAA | |
| F2 | TCAGACGTGTGCTCTTCCGATCTCTCGGTCATTTAGA | 10 |
| | GGAAGTAA | |
| F3 | TCAGACGTGTGCTCTTCCGATCTCTTGGTCATTTAGA | 11 |
| | GGAACTAA | |
| F4 | TCAGACGTGTGCTCTTCCGATCTCCCGGTCATTTAGA | 12 |
| | GGAAGTAA | |
| F5 | TCAGACGTGTGCTCTTCCGATCTCTAGGCTATTTAGA | 13 |
| | GGAAGTAA | |
| F6 | TCAGACGTGTGCTCTTCCGATCTCTTAGTTATTTAGA | 14 |
| | GGAAGTAA | |
| F7 | TCAGACGTGTGCTCTTCCGATCTCTACGTCATTTAGA | 15 |
| | GGAAGTAA | |
| F8 | TCAGACGTGTGCTCTTCCGATCTCTTGGTCATTTAGA | 16 |
| | GGTCGTAA | |
| R9 | TCAGACGTGTGCTCTTCCGATCTGCTGCGTTCTTCAT | 17 |
| | CGATGC | |
| R10 | TCAGACGTGTGCTCTTCCGATCTGCTGCGTTCTTCAT | 18 |
| | CGATGG | |
| R11 | TCAGACGTGTGCTCTTCCGATCTGCTACGTTCTTCAT | 19 |
| | CGATGC | |
| R12 | TCAGACGTGTGCTCTTCCGATCTGCTGCGTTCTTCAT | 20 |
| | CGATGT | |
| R13 | TCAGACGTGTGCTCTTCCGATCTACTGTGTTCTTCAT | 21 |
| | CGATGT | |
| R14 | TCAGACGTGTGCTCTTCCGATCTGCTGCGTTCTTCAT | 22 |
| | CGTTGC | |
| R15 | TCAGACGTGTGCTCTTCCGATCTGCGTTCTTCATCGA | 23 |
| | TGC | |
| 16-349 | /5Phos/ | 24 |
| | GATCGGAAGAGCACACGTCTGAACTCCAGTCAC | |
| | /3Phos/ | |
| 13-691 | /5SpC3/ | 25 |
| | G*A*TCTACACTCTTTCCCTACACGACGCTCTTCCGA | |
| | TCT | |
| 18-214 | ATCTCGGTGGTCGCCGTATCAT /3SpC3/ | 26 |
| 18-215 | ATCTCGTATGCCGTCTTCTGCTTG /3SpC3/ | 27 |
| 18-204 | TTTTTTTTTTTTrUrUrUrUAATGATACGGCG | 28 |
| 18-205 | TTTTTTTTTTTTrUrUrUrUCAAGCAGAAGAC | 29 |
| 18-206 | /5Phos/ ACCACCGAGATCTACAC TATAGCCT | 30 |
| | ACACTCTTTCCCTACACGAC | |
| 18-207 | /5Phos/ ACCACCGAGATCTACAC ATAGAGGC | 31 |
| | ACACTCTTTCCCTACACGAC | |
| 18-210 | /5Phos/ GGCATACGAGAT CGAGTAAT | 32 |
| | GTGACTGGAGTTCAGACGTGT | |
| 18-211 | /5Phos/ GGCATACGAGAT TCTCCGGA | 33 |
| | GTGACTGGAGTTCAGACGTGT | |
| 18-212 | /5Phos/ GGCATACGAGAT AATGAGCG | 34 |
| | GTGACTGGAGTTCAGACGTGT | |
| 18-213 | /5Phos/ GGCATACGAGAT GGAATCTC | 35 |
| | GTGACTGGAGTTCAGACGTGT | |
| 13-690 | /Phos/AGATTCGGAAGAGCACACGTCTGAACTCCAGT | 36 |
| | C*A*C/3SpC3/ | |
| 13-712 | AGACGUGUGCUCUTCCGATC/3′-dT/ | 37 |
| 14-161 | AATGATACGGCGACCACCGAGATCTACACTATAGCC | 38 |
| | TACACTCTTTCCCTACACGAC | |
| 14-882 | TCAGACGUGUGCUCUUCCGAU*C*U | 3 |
| 17-277 | CAAGCAGAAGACGGCATACGAGATCTGTGTTGGTGA | 39 |
| | CTGGAGTTCAGACGTG | |
| 18-452 | TTTTTTTTTTTTrUrUrUrUCAAGCAGAAGACGGCATAC | 40 |
| | GAGATCTATCGCTGTGACTGGAGTTCAGACGTGTGC | |
| | TCTTCCGATCT | |
| 18-453 | TTTTTTTTTTTTrUrUrUrUAATGATACGGCGACCACCG | 41 |
| | AGATCTACACTATAGCCTACACTCTTTCCCTACACGA | |
| | CGCTCTTCCGATCT | |
| 19-31 | ACACTCTTTCCCTACACGACGCTCTTCCGATCT | 42 |
| 19-34 | AATGATACGGCGACCACCGAGATCTACACAGACTGC | 43 |
| | GAA ACACTCTTTCCCTACACGACGCTCTTCCGATCT | |
| 19-37 | CAAGCAGAAGACGGCATACGAGATCACCTATGCCGT | 44 |
| | GACTGGAGTTCAGACGTGTGCTCTTCCGATCT | |
| 19-351 | GTCGTGTAGGGAAAGAGTGTTTT/iSPC3/ACACTCTTT | 45 and 42, |
| | CCCTACACGACGCTCTTCCGATCT | respectively |
| 19-352 | GAAGAGCACACGTCUGAACUCCAGTCAC/3SpC3/ | 46 |
| 19-353 | AGATCGGAAGAGCACACGTCUGAACUCCAGTCAC/ | 47 |
| | 3SpC3/ | |
| 19-354 | AGAUCGGAAGAGCACACGTCUGAACUCCAGTCAC/ | 48 |
| | 3SpC3/ | |
| 19-356 | AGAUCGGAAGAGTACACGTCUGAACUCCAGTCACTT | 49 |
| | T | |
| 19-357 | AGAUCGGAAGAGTACATGTCUGAACUCCAGTCACTT | 50 |
| | T | |
| 20-141 | AGACGUGUGCUCUUCCGATCU | 51 |
| 20-146 | AGACGUGUGCUCUUCCGAUCT | 52 |
| 20-147 | GTCGTGTAGGGAAAGAGTGTTTT/SpC3/ACACTCTTT | 45 and 53, |
| | CCCTACACGACGCTCTTCCGAT | respectively |
| 20-148 | 5Phos/GATCGGAAGAGCACACGTCTGAACTCCAGTCA | 54 |
| | C/3Phos/ | |
| Pl | TCAGACGTGTGCTCTTCCGATCTCCTACGGGAGGCA | 55 |
| | GCAG | |
| P2 | TCAGACGTGTGCTCTTCCGATCTCTACCAGGGTATCT | 56 |
| | AATCC | |
| P3 | TCAGACGTGTGCTCTTCCGATCTGCCAGCAGCCGCG | 57 |
| | GTAA | |
| P4 | TCAGACGTGTGCTCTTCCGATCTCCGTCAATTCMTTT | 58 |
| | GAGTTT | |
| P5 | TCAGACGTGTGCTCTTCCGATCTGYAACGAGCGCAA | 59 |
| | CCC | |
| P6 | TCAGACGTGTGCTCTTCCGATCTGACGGGCGGTGTG | 60 |
| | TACAA | |
| P7 | TCAGACGTGTGCTCTTCCGATCTATGGCTGTCGTCAG | 61 |
| | CT | |
| P8 | TCAGACGTGTGCTCTTCCGATCTTACCTTGTTACGAC | 62 |
| | TT | |
| P9 | TCAGACGTGTGCTCTTCCGATCTGAGTTTGATCMTG | 63 |
| | GCTCAG | |
| P10 | TCAGACGTGTGCTCTTCCGATCTCGTCAGGCTTTCGC | 64 |
| | CCATTG | |
| P11 | TCAGACGTGTGCTCTTCCGATCTGTTCAGGCTTCCGC | 65 |
| | CCATTG | |
| P12 | TCAGACGTGTGCTCTTCCGATCTGGTCAGGCTTTCGC | 66 |
| | CCATTG | |
| P13 | TCAGACGTGTGCTCTTCCGATCTGATCAGGCTTTCGC | 67 |
| | CCATTG | |
| P14 | TCAGACGTGTGCTCTTCCGATCTGGTCAGACTTTCGT | 68 |
| | CCATTG | |
| P15 | TCAGACGTGTGCTCTTCCGATCTGATCAGGGTTTCCC | 69 |
| | CCATTG | |
| P16 | TCAGACGTGTGCTCTTCCGATCTCATCAGAGTTGCCT | 70 |
| | CCATTG | |
| P17 | TCAGACGTGTGCTCTTCCGATCTCGTCAGGCTTGCGC | 71 |
| | CCATTG | |
| P18 | TCAGACGTGTGCTCTTCCGATCTCAACGCGAAGAAC | 72 |
| | CTTACC | |
| P19 | TCAGACGTGTGCTCTTCCGATCTATACGCGAGGAAC | 73 |
| | CTTACC | |
| P20 | TCAGACGTGTGCTCTTCCGATCTCTAACCGANGAAC | 74 |
| | CTYACC | |
| P21 | TCAGACGTGTGCTCTTCCGATCTCAACGCGAAAAAC | 75 |
| | CTTACC | |
| P22 | TCAGACGTGTGCTCTTCCGATCTCAACGCGCAGAAC | 76 |
| | CTTACC | |
| P23 | TCAGACGTGTGCTCTTCCGATCTATACGCGARGAAC | 77 |
| | CTTACC | |

Abbreviations used for oligonucleotide modifications:
/5Phos/ 5′ phosphate group
/3SpC3/ C3 spacer, preventing the 3′ end from extension by a DNA polymerase or ligation by a DNA ligase
/5SpC3/ C3 spacer, preventing 5′ end from ligation and degradation
/iSpC3/ internal C3 spacer, preventing continuous replication by a DNA polymerase
* a nuclease-resistant, phosphorothioate bond preventing the 3′ end from degradation

TABLE 2
Quantification of the yield of the NGS libraries prepared by ligation-
coupled PCR method in the presence of hairpin or linear 5′ adapter

| | | Average Molarity (nM) |
|---|---|---|
| Ligation/PCR | Qubit | quantified by the Agilent |
| Reaction Condition | (ng/μl) | Bioanalyzer |
| Truncated hairpin 5′ adapter | 5.58 | 29.35 |
| with an un-replicable spacer | 5.54 | |
| Truncated linear 5′ adapter | 3.75 | 15.75 |
| | 3.34 | |
| Indexing primers added post- | 5.23 | 27.35 |
| ligation | 4.94 | |

TABLE 3
Quantification of the yield of the NGS libraries prepared by ligation-
coupled PCR method in the presence of different primer blockers

| | | Average Molarity (nM) |
|---|---|---|
| Ligation/PCR | Qubit | Quantified by the Agilent |
| Reaction Condition | (ng/μl) | Bioanalyzer |
| No blocker | 1.47 | 6.58 |
| | 1.58 | |
| Blocker_1 (19-356) | 5.98 | 28.84 |
| | 5.67 | |
| Blocker_2 (19-357) | 3.8 | 16.26 |
| | 3.7 | |
| Blocker_3 (19-352) | 3.84 | 16.93 |
| | 4.03 | |
| Blocker_4 (19-353) | 3.67 | 16.12 |
| | 3.65 | |
| Blocker_5 (19-354) | 3.62 | 15.95 |
| | 3.5 | |
| Indexing primers added | 5.23 | 27.35 |
| post-ligation | 4.94 | |

TABLE 4
Quantification of the yield of the NGS libraries prepared by ligation-
coupled PCR method when using 3′ adapters with T or U overhang

| | | DNA | Molarity (nM) |
|---|---|---|---|
| NGS | | input | quantified by the |
| library | Scheme | (ng) | Agilent Bioanalyzer |
| Library | Ligation of the 3′ adapter with T | 50 | 9.3 |
| prep A | overhang followed by the ligation- | | 9 |
| | coupled PCR with hairpin 5′ adapter | 100 | 17.5 |
| | and truncated indexing primers | | 15.4 |
| | | 250 | 32 |
| | | | 31.7 |
| Library | Ligation of the 3′ adapter with U | 50 | 6.7 |
| prep B | overhang followed by the ligation- | | 6.7 |
| | coupled PCR with hairpin 5′ adapter | 100 | 12.8 |
| | and truncated indexing primers | | 11.3 |
| | | 250 | 26.4 |
| | | | 25 |

TABLE 5
Sequencing metrics of NGS libraries prepared by ligation-coupled
PCR method when using 3′ adapters with T or U overhang

| | DNA | | | | | |
|---|---|---|---|---|---|---|
| NGS | Input | Estimated | Mean Aligned | | | % Aligned |
| library | (ng) | Library Size | Insert | % Chimera | % Duplicates | Reads |
| Library | 50 | 1,210,601,387 | 200 | 1.6 | 0.08 | 99.6 |
| Prep A | | 1,057,670,303 | 200 | 1.7 | 0.08 | 99.6 |
| | 100 | 1,208,874,574 | 203 | 2.7 | 0.07 | 99.5 |
| | | 1,031,607,179 | 199 | 2.6 | 0.08 | 99.5 |
| | 250 | 907,367,064 | 199 | 6.8 | 0.09 | 99.4 |
| | | 965,921,523 | 202 | 8.5 | 0.09 | 99.2 |
| Library | 50 | 939,292,885 | 201 | 1.5 | 0.08 | 99.6 |
| Prep B | | 951,672,989 | 201 | 1.7 | 0.08 | 99.6 |
| | 100 | 945,015,752 | 206 | 2.4 | 0.09 | 99.5 |
| | | 926,181,356 | 201 | 2.9 | 0.08 | 99.5 |
| | 250 | 841,055,892 | 205 | 7.3 | 0.1 | 99.2 |
| | | 743,955,829 | 204 | 7.6 | 0.12 | 99.1 |

TABLE 6
Quantification of the yield of NGS library prepared
by ligation-coupled PCR method when using 3′
adapter with T overhang in picogram DNA input range

| | DNA | | | |
|---|---|---|---|---|
| Sample | input | #PCR | Molarity | Ave size |
| # | (pg) | cycles | (nM) | (bp) |
| 1 | 100 | 12 | 4 | 373 |
| 2 | | | 4.42 | 380 |
| 3 | 6.6 | 17 | 10.6 | 379 |
| 4 | | | 7.4 | 385 |
| 5 | 3.3 | 17 | 6.2 | 375 |
| 6 | | | 6.2 | 377 |

TABLE 7
Quantification of the yield of NGS library prepared
by ligation-coupled PCR method when using 3′
adapter with T overhang in femtogram DNA input range

| | | Spiked-in | | | |
|---|---|---|---|---|---|
| | DNA | carrier | | | |
| | Input | ssDNA | #PCR | Qubit | Ave Size |
| Sample # | (fg) | (pg) | cycles | (ng/ul) | (bp) |
| 1 | 3,300 | 3.3 | 17 | 1.2 | 380 |
| 2 | | 0 | | 1.4 | 384 |
| 3 | 300 | 3.3 | 21 | 30.5 | 416 |
| 4 | | 0 | | 24.4 | 411 |
| 5 | | 3.3 | 24 | 23.6 | 420 |
| 6 | 30 | 0 | | 21 | 409 |
| 7 | 3 | 3.3 | 27 | 24.3 | 517 |
| 8 | | 0 | | 23.9 | 495 |
| 9 | 0.3 | 3.3 | 30 | 25 | 497 |
| 10 | | 0 | | 20 | 509 |

1. A method for ligation-coupled polymerase chain reaction (PCR), comprising:
(i) providing a partially double-stranded DNA substrate comprising a first strand and a second strand, the partially double-stranded DNA substrate comprising a first 3′ overhang, a double-stranded portion, and a second 3′ overhang,
the first strand comprising, in a 5′ to 3′ direction: a first 5′ end, a first portion, and a second portion,
the second strand comprising, in a 5′ to 3′ direction: a second 5′ end, a third portion, and a fourth portion of the partially double-stranded DNA substrate,
wherein the first portion of the first strand and the third portion of the second strand are complementary and form the double-stranded portion,
wherein the second portion of the first strand forms the first 3′ overhang,
wherein the fourth portion of the second strand forms the second 3′ overhang,
wherein the second portion of the first strand and the fourth portion of the second strand each comprise a first common nucleotide sequence positioned at a 5′ end of the first 3′ overhang and the second 3′ overhang, respectively, and
wherein the second portion of the first strand and the fourth portion of the second strand each comprise a second common nucleotide sequence positioned 3′ to the first common nucleotide sequence;
(ii) adding a plurality of first indexing primers, a plurality of second indexing primers, a ligase, a DNA polymerase and deoxynucleotide triphosphates (dNTPs) to the partially double-stranded DNA substrate to yield a first reaction mixture, wherein each of the plurality of first indexing primers comprise a first 3′ terminal portion complementary to the first common nucleotide sequence, and wherein each of the plurality of second indexing primers comprise a second 3′ terminal portion complementary to the first common nucleotide sequence and a first 5′ portion positioned 5′ to the second 3′ terminal portion and complementary to the second common nucleotide sequence;
(iii) incubating the first reaction mixture under a first set of conditions comprising a ligation temperature for a ligation duration, wherein the first set of conditions is sufficient:
a) for the first 3′ terminal portion to anneal to the first common nucleotide sequence, and
b) for the ligase to ligate one of the plurality of first indexing primers to the first 5′ end of the first strand and one of the plurality of first indexing primers to the second 5′ end of the second strand to yield a second reaction mixture comprising:
a third strand comprising, in a 5′ to 3′ direction, one of the plurality of first indexing primers, the first portion, and the second portion, and
a fourth strand comprising, in a 5′ to 3′ direction, one of the plurality of first indexing primers, the third portion, and the fourth portion;
(iv) incubating the second reaction mixture under a second set of conditions comprising a first denaturation temperature for a first denaturation duration, a first annealing temperature for a first annealing duration, and a first extension temperature for a first extension duration, wherein the second set of conditions is sufficient:
a) to inactivate the ligase, denature double-stranded DNA, and, optionally, to activate the DNA polymerase,
b) for the second 3′ terminal portion and the first 5′ portion of one of the plurality of second indexing primers to anneal to at least the first common nucleotide sequence and the second common nucleotide sequence of the second portion of the third strand and for the second 3′ terminal portion and the first 5′ portion of one of the plurality of second indexing primers to anneal to at least the first common nucleotide sequence and the second common nucleotide sequence of the fourth portion of the fourth strand, and
c) for the DNA polymerase to extend the one of the plurality of second indexing primers annealed to the first common nucleotide sequence and second common nucleotide sequence of the second portion of the third strand and for the DNA polymerase to extend the one of the plurality of second indexing primers annealed to the first common nucleotide sequence and second common nucleotide sequence of the fourth portion of the fourth strand, to yield a third reaction mixture comprising the third strand, the fourth strand, a fifth strand, and a sixth strand, the fifth strand comprising, in a 5′ to 3′ direction, one of the plurality of second indexing primers, the third portion, and a reverse complement of one of the plurality of first indexing primers, the sixth strand comprising, in a 5′ to 3′ direction, one of the plurality of second indexing primers, the first portion, and the reverse complement of one of the plurality of first indexing primers;
(v) incubating the third reaction mixture under a third set of conditions comprising a second denaturation temperature for a second denaturation duration, a second annealing temperature for a second annealing duration, and a second extension temperature for a second extension duration, wherein the third set of conditions is sufficient
a) to denature double-stranded DNA,
b) for one of the plurality of first indexing primers to anneal to the reverse complement of one of the plurality of first indexing primers of the fifth strand and for one of the plurality of first indexing primers to anneal to the reverse complement of one of the plurality of first indexing primers of the sixth strand, and
c) for the DNA polymerase to extend the one of the plurality of first indexing primers annealed to the reverse complement of one of the plurality of first indexing primers of the fifth strand and the one of the plurality of first indexing primers annealed to the reverse complement of the one of the plurality of first indexing primers of the sixth strand, to yield a fourth reaction mixture comprising the fifth strand, the sixth strand, a seventh strand, and an eighth strand, the seventh strand comprising, in a 5′ to 3′ direction, one of the plurality of first indexing primers, the first portion, and a reverse complement of one of the plurality of second indexing primers, the eighth strand comprising, in a 5′ to 3′ direction, one of the plurality of first indexing primers, the third portion, and the reverse complement of one of the plurality of second indexing primers, wherein the seventh strand is complementary to the fifth strand, and wherein the eighth strand is complementary to the sixth strand; and
(vi) incubating the fourth reaction mixture under a fourth set of conditions comprising a third denaturation temperature for a third denaturation duration, a third annealing temperature for a third annealing duration, and a third extension temperature for a third extension duration, wherein the fourth set of conditions is sufficient for at least a portion of the plurality of first indexing primers and at least a portion of the plurality of second indexing primers to amplify the fifth strand and seventh strand and the sixth strand and eighth strand.

2. The method of claim 1, wherein steps (i) through (vi) are performed in a single closed tube.

3. The method of claim 1, wherein the partially double-stranded DNA substrate has a length of about 24 bases to about 6000 bases, wherein the first portion and the third portion of the first strand and the second strand, respectively, each have a length of about 20 bases to about 6000 bases, wherein the second portion of the first strand and the fourth portion of the second strand each have a length of about 4 bases to about 100 bases, wherein the first common nucleotide sequence has a length of about 1 base to about 50 bases, wherein the second common nucleotide sequence has a length from about 5 bases to about 30 bases, and wherein each of the plurality of first indexing primers has a length from about 20 bases to about 100 bases, and wherein each of the plurality of second indexing primers has a length from about 20 bases to about 100 bases.

4. (canceled)

5. (canceled)

6. (canceled)

7. (canceled)

8. (canceled)

9. (canceled)

10. (canceled)

11. The method of claim 1, wherein the first common nucleotide sequence and the first 3′ terminal portion have a melting temperature (Tₘ) greater than the ligation temperature, wherein the ligation temperature is less than a melting temperature (Tₘ) of the partially double-stranded DNA substrate, wherein a melting temperature (Tₘ) of the third strand and fourth strand is less than the first denaturing temperature, and wherein the first common nucleotide sequence, the first 3′ terminal portion, and the second 3′ terminal portion have a Tₘ less than the first annealing temperature.

12. (canceled)

13. (canceled)

14. (canceled)

15. (canceled)

16. (canceled)

17. (canceled)

18. (canceled)

19. (canceled)

20. The method of claim 1, wherein the ligation temperature is from about 25° C. to about 40° C.

21. The method of claim 1, wherein the ligation duration is from about 5 minutes to about 60 minutes.

22. (canceled)

23. The method of claim 1, wherein the ligase is a thermolabile ligase capable of ligation in a low magnesium buffer.

24. The method of claim 1, wherein the ligase is T3 DNA ligase.

25. The method of claim 24, wherein the ligase is added at about 30 to about 300 enzyme units per 50 μL of the first reaction mixture.

26. The method of claim 1, wherein the ligase is temperature-sensitive, and wherein the first denaturation temperature for the first denaturation duration in step (iv)(a) is sufficient to inactivate the ligase.

27. The method of claim 1, wherein the DNA polymerase is a thermostable DNA polymerase with 3′-5′ exonuclease proofreading activity selected from the group consisting of Kapa HiFi DNA Polymerase (Roche), NEB Q5 DNA Polymerase (NEB), PrimeStar GXL DNA Polymerase (Takara) and High Fidelity DNA Polymerase (Qiagen).

28. The method of claim 1, wherein the DNA polymerase is not active at the ligation temperature.

29. The method of claim 28, wherein the DNA polymerase further comprises a hot start antibody or aptamer, and wherein the hot start antibody or aptamer increases an activation temperature of the DNA polymerase.

30. The method of claim 29, wherein the DNA polymerase is selected from the group consisting of Kapa HiFi Hot Start DNA Polymerase (Roche), NEB Q5 Hot Start DNA Polymerase (NEB), PrimeStar GXL Hot Start DNA Polymerase (Takara) and High Fidelity Hot Start DNA Polymerase (Qiagen).

31. The method of claim 1, wherein the DNA polymerase is a hot start polymerase, and wherein the first denaturation temperature for the first denaturation duration in step (iv)(a) is sufficient to activate the hot start polymerase.

32. The method of claim 1, wherein a melting temperature (Tₘ) of each of the plurality of second indexing primers and the second portion or the fourth portion of the first strand and second strand, respectively, is higher than a Tₘ of each of the plurality of first indexing primers and the second portion or the fourth portion of the first strand and the second strand.

33. The method of claim 1, wherein the first denaturation temperature, the second denaturation temperature, and the third denaturation temperature are each independently about 95° C. to about 98° C., and wherein the first denaturation duration, the second denaturation duration, and the third denaturation duration are each independently from about 30 seconds to about 2 minutes.

34. (canceled)

35. The method of claim 1, wherein the first annealing temperature, the second annealing temperature, and the third annealing temperature are each independently about 55° C. to about 65° C., and wherein the first annealing duration, the second annealing duration, and the third annealing duration are each independently about 10 seconds to about 60 seconds.

36. (canceled)

37. The method of claim 1, wherein the first extension temperature, the second extension temperature, and the third extension temperature are each independently about 62° C. to about 72° C., and wherein the first extension duration, the second extension duration, and the third extension duration are each independently about 30 seconds to about 5 minutes.

38. (canceled)

39. The method of claim 1, wherein the plurality of first indexing primers and the plurality of second indexing primers are each independently added to the first reaction mixture at about 100 nM to about 1 μM.

40-68. (canceled)

69. The method of claim 1 further comprising sequencing the fifth strand and seventh strand or the sixth strand and the eighth strand.

70. The method of claim 1, wherein each of the plurality of first indexing primers comprises a first 5′ terminal portion and each of the plurality of second indexing primers further comprises a second 5′ terminal portion, wherein each of the first 5′ terminal portion and the second 5′ terminal portion comprise, in a 5′ to 3′ direction, a first sequence comprising two or more deoxynucleotides and a second sequence comprising three or more ribonucleotides, wherein the DNA polymerase has 3′ to 5′ exonuclease activity, whereby the fifth strand and sixth strand further comprise the second 5′ terminal portion at a 5′ end of the fifth strand and sixth strand, and whereby the seventh strand and eighth strand further comprise the first 5′ terminal portion at a 5′ end of the seventh strand and eighth strand, and whereby the fifth strand and seventh strand can form a first double-stranded product having a first 5′ overhang and a second 5′ overhang, and whereby the sixth strand and eighth strand can form a second double-stranded product having a third 5′ overhang and a fourth 5′ overhang, the method further comprising:
adding a probe complementary to each of the first 5′ overhang, second 5′ overhang, third 5′ overhang and fourth 5′ overhang in an amount sufficient to yield a target molar quantity of the fifth strand, sixth strand, seventh strand and eighth strand, and a second ligase where the fifth strand, sixth strand, seventh strand and eighth strand are present in an amount greater than the target molar quantity, wherein the probe comprises a modification to provide resistance to digestion by an enzyme with 3′ exocnuclease activity;
incubating the fifth strand, sixth strand, seventh strand, eighth strand, second ligase 3 and probe under conditions sufficient for the probe to ligate to the target molar quantity of the fifth strand, sixth strand, seventh strand and eighth strand to yield a pre-normalization reaction mixture;
adding an enzyme with 3′ exonuclease activity to the pre-normalization reaction mixture;
incubating the pre-normalization reaction mixture and enzyme with exonuclease activity under conditions sufficient for the enzyme with 3′ exonuclease activity to digest the fifth strand sixth strand, seventh strand, and eighth strand not ligated to the probe, to yield a normalized next generation sequencing (NGS) library.

71. The method of claim 70, further comprising sequencing the normalized NGS library.

72-252. (canceled)


SEQUENCE LISTING (127 sequences)

SEQ ID NO: 1 - DNA, 16 bp, synthetic construct
  misc_feature 1..12: note = DNA
  source 1..16: mol_type = other DNA
  misc_feature 13..16: note = RNA
tttttttttttttttt

SEQ ID NO: 2 - DNA, 12 bp, synthetic construct
  source 1..12: mol_type = other DNA
tttttttttttt

SEQ ID NO: 3 - DNA, 23 bp, synthetic construct
  misc_feature order(8,10,13,15..16,21,23): note = RNA
  misc_feature order(1..7,9,11..12,14,17..20,22): note = DNA
  source 1..23: mol_type = other DNA
tcagacgtgtgctcttccgatct

SEQ ID NO: 4 - DNA, 86 bp, synthetic construct
  misc_feature order(1..12,17..86): note = DNA
  misc_feature 13..16: note = RNA
  source 1..86: mol_type = other DNA
ttttttttttttttttaatgatacggcgaccaccgagatctacacgtactgacacactctttccctacacgacgctcttccgatct

SEQ ID NO: 5 - DNA, 82 bp, synthetic construct
  misc_feature 13..16: note = RNA
  misc_feature order(1..12,17..82): note = DNA
  source 1..82: mol_type = other DNA
ttttttttttttttttcaagcagaagacggcatacgagatgcgcgagagtgactggagttcagacgtgtgctcttccgatct

SEQ ID NO: 6 - DNA, 38 bp, synthetic construct
  misc_feature order(1..12,17..38): note = DNA
  misc_feature 13..16: note = RNA
  source 1..38: mol_type = other DNA
ttttttttttttttttaatgatacggcgaccaccgaga

SEQ ID NO: 7 - DNA, 40 bp, synthetic construct
  misc_feature 13..16: note = RNA
  misc_feature order(1..12,17..40): note = DNA
  source 1..40: mol_type = other DNA
ttttttttttttttttcaagcagaagacggcatacgagat

SEQ ID NO: 8 - DNA, 36 bp, synthetic construct
  source 1..36: mol_type = other DNA
agatcggaagagcttttttacacgtctgaactcctt

SEQ ID NO: 9 - DNA, 45 bp, synthetic construct
  source 1..45: mol_type = other DNA
tcagacgtgtgctcttccgatctcttggtcatttagaggaagtaa

SEQ ID NO: 10 - DNA, 45 bp, synthetic construct
  source 1..45: mol_type = other DNA
tcagacgtgtgctcttccgatctctcggtcatttagaggaagtaa

SEQ ID NO: 11 - DNA, 45 bp, synthetic construct
  source 1..45: mol_type = other DNA
tcagacgtgtgctcttccgatctcttggtcatttagaggaactaa

SEQ ID NO: 12 - DNA, 45 bp, synthetic construct
  source 1..45: mol_type = other DNA
tcagacgtgtgctcttccgatctcccggtcatttagaggaagtaa

SEQ ID NO: 13 - DNA, 45 bp, synthetic construct
  source 1..45: mol_type = other DNA
tcagacgtgtgctcttccgatctctaggctatttagaggaagtaa

SEQ ID NO: 14 - DNA, 45 bp, synthetic construct
  source 1..45: mol_type = other DNA
tcagacgtgtgctcttccgatctcttagttatttagaggaagtaa

SEQ ID NO: 15 - DNA, 45 bp, synthetic construct
  source 1..45: mol_type = other DNA
tcagacgtgtgctcttccgatctctacgtcatttagaggaagtaa

SEQ ID NO: 16 - DNA, 45 bp, synthetic construct
  source 1..45: mol_type = other DNA
tcagacgtgtgctcttccgatctcttggtcatttagaggtcgtaa

SEQ ID NO: 17 - DNA, 43 bp, synthetic construct
  source 1..43: mol_type = other DNA
tcagacgtgtgctcttccgatctgctgcgttcttcatcgatgc

SEQ ID NO: 18 - DNA, 43 bp, synthetic construct
  source 1..43: mol_type = other DNA
tcagacgtgtgctcttccgatctgctgcgttcttcatcgatgg

SEQ ID NO: 19 - DNA, 43 bp, synthetic construct
  source 1..43: mol_type = other DNA
tcagacgtgtgctcttccgatctgctacgttcttcatcgatgc

SEQ ID NO: 20 - DNA, 43 bp, synthetic construct
  source 1..43: mol_type = other DNA
tcagacgtgtgctcttccgatctgctgcgttcttcatcgatgt

SEQ ID NO: 21 - DNA, 43 bp, synthetic construct
  source 1..43: mol_type = other DNA
tcagacgtgtgctcttccgatctactgtgttcttcatcgatgt

SEQ ID NO: 22 - DNA, 43 bp, synthetic construct
  source 1..43: mol_type = other DNA
tcagacgtgtgctcttccgatctgctgcgttcttcatcgttgc

SEQ ID NO: 23 - DNA, 40 bp, synthetic construct
  source 1..40: mol_type = other DNA
tcagacgtgtgctcttccgatctgcgttcttcatcgatgc

SEQ ID NO: 24 - DNA, 33 bp, synthetic construct
  source 1..33: mol_type = other DNA
gatcggaagagcacacgtctgaactccagtcac

SEQ ID NO: 25 - DNA, 38 bp, synthetic construct
  source 1..38: mol_type = other DNA
gatctacactctttccctacacgacgctcttccgatct

SEQ ID NO: 26 - DNA, 22 bp, synthetic construct
  source 1..22: mol_type = other DNA
atctcggtggtcgccgtatcat

SEQ ID NO: 27 - DNA, 24 bp, synthetic construct
  source 1..24: mol_type = other DNA
atctcgtatgccgtcttctgcttg

SEQ ID NO: 28 - DNA, 28 bp, synthetic construct
  misc_feature order(1..12,17..28): note = DNA
  misc_feature 13..16: note = RNA
  source 1..28: mol_type = other DNA
ttttttttttttttttaatgatacggcg

SEQ ID NO: 29 - DNA, 28 bp, synthetic construct
  misc_feature 13..16: note = RNA
  misc_feature order(1..12,17..28): note = DNA
  source 1..28: mol_type = other DNA
ttttttttttttttttcaagcagaagac

SEQ ID NO: 30 - DNA, 45 bp, synthetic construct
  source 1..45: mol_type = other DNA
accaccgagatctacactatagcctacactctttccctacacgac

SEQ ID NO: 31 - DNA, 45 bp, synthetic construct
  source 1..45: mol_type = other DNA
accaccgagatctacacatagaggcacactctttccctacacgac

SEQ ID NO: 32 - DNA, 41 bp, synthetic construct
  source 1..41: mol_type = other DNA
ggcatacgagatcgagtaatgtgactggagttcagacgtgt

SEQ ID NO: 33 - DNA, 41 bp, synthetic construct
  source 1..41: mol_type = other DNA
ggcatacgagattctccggagtgactggagttcagacgtgt

SEQ ID NO: 34 - DNA, 41 bp, synthetic construct
  source 1..41: mol_type = other DNA
ggcatacgagataatgagcggtgactggagttcagacgtgt

SEQ ID NO: 35 - DNA, 41 bp, synthetic construct
  source 1..41: mol_type = other DNA
ggcatacgagatggaatctcgtgactggagttcagacgtgt

SEQ ID NO: 36 - DNA, 35 bp, synthetic construct
  source 1..35: mol_type = other DNA
agattcggaagagcacacgtctgaactccagtcac

SEQ ID NO: 37 - DNA, 21 bp, synthetic construct
  misc_feature order(6,8,11,13): note = RNA
  misc_feature order(1..5,7,9..10,12,14..21): note = DNA
  source 1..21: mol_type = other DNA
agacgtgtgctcttccgatct

SEQ ID NO: 38 - DNA, 57 bp, synthetic construct
  source 1..57: mol_type = other DNA
aatgatacggcgaccaccgagatctacactatagcctacactctttccctacacgac

SEQ ID NO: 39 - DNA, 52 bp, synthetic construct
  source 1..52: mol_type = other DNA
caagcagaagacggcatacgagatctgtgttggtgactggagttcagacgtg

SEQ ID NO: 40 - DNA, 82 bp, synthetic construct
  misc_feature order(1..12,17..82): note = DNA
  misc_feature 13..16: note = RNA
  source 1..82: mol_type = other DNA
ttttttttttttttttcaagcagaagacggcatacgagatctatcgctgtgactggagttcagacgtgtgctcttccgatct

SEQ ID NO: 41 - DNA, 86 bp, synthetic construct
  misc_feature order(1..12,17..86): note = DNA
  misc_feature 13..16: note = RNA
  source 1..86: mol_type = other DNA
ttttttttttttttttaatgatacggcgaccaccgagatctacactatagcctacactctttccctacacgacgctcttccgatct

SEQ ID NO: 42 - DNA, 33 bp, synthetic construct
  source 1..33: mol_type = other DNA
acactctttccctacacgacgctcttccgatct

SEQ ID NO: 43 - DNA, 72 bp, synthetic construct
  source 1..72: mol_type = other DNA
aatgatacggcgaccaccgagatctacacagactgcgaaacactctttccctacacgacgctcttccgatct

SEQ ID NO: 44 - DNA, 68 bp, synthetic construct
  source 1..68: mol_type = other DNA
caagcagaagacggcatacgagatcacctatgccgtgactggagttcagacgtgtgctcttccgatct

SEQ ID NO: 45 - DNA, 23 bp, synthetic construct
  source 1..23: mol_type = other DNA
gtcgtgtagggaaagagtgtttt

SEQ ID NO: 46 - DNA, 28 bp, synthetic construct
  misc_feature order(1..14,16..19,21..28): note = DNA
  misc_feature order(15,20): note = RNA
  source 1..28: mol_type = other DNA
gaagagcacacgtctgaactccagtcac

SEQ ID NO: 47 - DNA, 34 bp, synthetic construct
  misc_feature order(1..20,22..25,27..34): note = DNA
  misc_feature order(21,26): note = RNA
  source 1..34: mol_type = other DNA
agatcggaagagcacacgtctgaactccagtcac

SEQ ID NO: 48 - DNA, 34 bp, synthetic construct; identical to SEQ ID NO: 47
  misc_feature order(1..3,5..20,22..25,27..34): note = DNA
  misc_feature order(4,21,26): note = RNA
  source 1..34: mol_type = other DNA

SEQ ID NO: 49 - DNA, 37 bp, synthetic construct
  misc_feature order(1..3,5..20,22..25,27..37): note = DNA
  misc_feature order(4,21,26): note = RNA
  source 1..37: mol_type = other DNA
agatcggaagagtacacgtctgaactccagtcacttt

SEQ ID NO: 50 - DNA, 37 bp, synthetic construct
  misc_feature order(1..3,5..20,22..25,27..37): note = DNA
  misc_feature order(4,21,26): note = RNA
  source 1..37: mol_type = other DNA
agatcggaagagtacatgtctgaactccagtcacttt

SEQ ID NO: 51 - DNA, 21 bp, synthetic construct
  misc_feature order(1..5,7,9..10,12,15..20): note = DNA
  misc_feature order(6,8,11,13..14,21): note = RNA
  source 1..21: mol_type = other DNA
agacgtgtgctcttccgatct

SEQ ID NO: 52 - DNA, 21 bp, synthetic construct
  misc_feature order(1..5,7,9..10,12,15..18,20..21): note = DNA
  misc_feature order(6,8,11,13..14,19): note = RNA
  source 1..21: mol_type = other DNA
agacgtgtgctcttccgatct

SEQ ID NO: 53 - DNA, 31 bp, synthetic construct
  source 1..31: mol_type = other DNA
acactctttccctacacgacgctcttccgat

SEQ ID NO: 54 - DNA, 33 bp, synthetic construct; identical to SEQ ID NO: 24
  source 1..33: mol_type = other DNA

SEQ ID NO: 55 - DNA, 40 bp, synthetic construct
  source 1..40: mol_type = other DNA
tcagacgtgtgctcttccgatctcctacgggaggcagcag

SEQ ID NO: 56 - DNA, 42 bp, synthetic construct
  source 1..42: mol_type = other DNA
tcagacgtgtgctcttccgatctctaccagggtatctaatcc

SEQ ID NO: 57 - DNA, 40 bp, synthetic construct
  source 1..40: mol_type = other DNA
tcagacgtgtgctcttccgatctgccagcagccgcggtaa

SEQ ID NO: 58 - DNA, 43 bp, synthetic construct
  source 1..43: mol_type = other DNA
tcagacgtgtgctcttccgatctccgtcaattcmtttgagttt

SEQ ID NO: 59 - DNA, 39 bp, synthetic construct
  source 1..39: mol_type = other DNA
tcagacgtgtgctcttccgatctgyaacgagcgcaaccc

SEQ ID NO: 60 - DNA, 41 bp, synthetic construct
  source 1..41: mol_type = other DNA
tcagacgtgtgctcttccgatctgacgggcggtgtgtacaa

SEQ ID NO: 61 - DNA, 39 bp, synthetic construct
  source 1..39: mol_type = other DNA
tcagacgtgtgctcttccgatctatggctgtcgtcagct

SEQ ID NO: 62 - DNA, 39 bp, synthetic construct
  source 1..39: mol_type = other DNA
tcagacgtgtgctcttccgatcttaccttgttacgactt

SEQ ID NO: 63 - DNA, 42 bp, synthetic construct
  source 1..42: mol_type = other DNA
tcagacgtgtgctcttccgatctgagtttgatcmtggctcag

SEQ ID NO: 64 - DNA, 43 bp, synthetic construct
  source 1..43: mol_type = other DNA
tcagacgtgtgctcttccgatctcgtcaggctttcgcccattg

SEQ ID NO: 65 - DNA, 43 bp, synthetic construct
  source 1..43: mol_type = other DNA
tcagacgtgtgctcttccgatctgttcaggcttccgcccattg

SEQ ID NO: 66 - DNA, 43 bp, synthetic construct
  source 1..43: mol_type = other DNA
tcagacgtgtgctcttccgatctggtcaggctttcgcccattg

SEQ ID NO: 67 - DNA, 43 bp, synthetic construct
  source 1..43: mol_type = other DNA
tcagacgtgtgctcttccgatctgatcaggctttcgcccattg

SEQ ID NO: 68 - DNA, 43 bp, synthetic construct
  source 1..43: mol_type = other DNA
tcagacgtgtgctcttccgatctggtcagactttcgtccattg

SEQ ID NO: 69 - DNA, 43 bp, synthetic construct
  source 1..43: mol_type = other DNA
tcagacgtgtgctcttccgatctgatcagggtttcccccattg

SEQ ID NO: 70 - DNA, 43 bp, synthetic construct
  source 1..43: mol_type = other DNA
tcagacgtgtgctcttccgatctcatcagagttgcctccattg

SEQ ID NO: 71 - DNA, 43 bp, synthetic construct
  source 1..43: mol_type = other DNA
tcagacgtgtgctcttccgatctcgtcaggcttgcgcccattg

SEQ ID NO: 72 - DNA, 42 bp, synthetic construct
  source 1..42: mol_type = other DNA
tcagacgtgtgctcttccgatctcaacgcgaagaaccttacc

SEQ ID NO: 73 - DNA, 42 bp, synthetic construct
  source 1..42: mol_type = other DNA
tcagacgtgtgctcttccgatctatacgcgaggaaccttacc

SEQ ID NO: 74 - DNA, 42 bp, synthetic construct
  misc_difference 32: note = a, c, t, g, unknown or other
  source 1..42: mol_type = other DNA
tcagacgtgtgctcttccgatctctaaccgangaacctyacc

SEQ ID NO: 75 - DNA, 42 bp, synthetic construct
  source 1..42: mol_type = other DNA
tcagacgtgtgctcttccgatctcaacgcgaaaaaccttacc

SEQ ID NO: 76 - DNA, 42 bp, synthetic construct
  source 1..42: mol_type = other DNA
tcagacgtgtgctcttccgatctcaacgcgcagaaccttacc

SEQ ID NO: 77 - DNA, 42 bp, synthetic construct
  source 1..42: mol_type = other DNA
tcagacgtgtgctcttccgatctatacgcgargaaccttacc

SEQ ID NO: 78 - DNA, 24 bp, synthetic construct
  source 1..24: mol_type = other DNA
caagcagaagacggcatacgagat

SEQ ID NO: 79 - DNA, 34 bp, synthetic construct
  source 1..34: mol_type = other DNA
gtgactggagttcagacgtgtgctcttccgatct

SEQ ID NO: 80 - DNA, 34 bp, synthetic construct
  source 1..34: mol_type = other DNA
agatcggaagagataacgtctgaactccagtcac

SEQ ID NO: 81 - DNA, 46 bp, synthetic construct
  source 1..46: mol_type = other DNA
agatcggaagagcacacgtctgaactccagtcacccctggcgagcg

SEQ ID NO: 82 - DNA, 16 bp, synthetic construct
  source 1..16: mol_type = other DNA
ttttcgctcgccaggg

SEQ ID NO: 83 - DNA, 49 bp, synthetic construct
  source 1..49: mol_type = other DNA
agatcggaagagcacacgtctgaactccagtcactttccctggcgagcg

SEQ ID NO: 84 - DNA, 53 bp, synthetic construct
  source 1..53: mol_type = other DNA
ttttcgctcgccagggaaagtgactggagttcagacgtgtgctcttccgatct

SEQ ID NO: 85 - DNA, 23 bp, synthetic construct
  misc_feature order(8,10,13,15,21,23): note = RNA
  misc_feature order(1..7,9,11..12,14,16..20,22): note = DNA
  source 1..23: mol_type = other DNA
tcagacgtgtgctcttccgatct

SEQ ID NO: 86 - DNA, 23 bp, synthetic construct
  source 1..23: mol_type = other DNA
agatcggaagagcacacgtctga

SEQ ID NO: 87 - DNA, 29 bp, synthetic construct
  source 1..29: mol_type = other DNA
aatgatacggcgaccaccgagatctacac

SEQ ID NO: 88 - DNA, 37 bp, synthetic construct
  source 1..37: mol_type = other DNA
agatcggaagagataacgtctgaactccagtcacttt

SEQ ID NO: 89 - DNA, 34 bp, synthetic construct
  source 1..34: mol_type = other DNA
agatcggaagagcttttttacacgtctgaactcc

SEQ ID NO: 90 - DNA, 37 bp, synthetic construct
  modified_base order(4,21,26): mod_base = OTHER; note = Abasic nucleotide
  source 1..37: mol_type = other DNA
agancggaagagtacacgtcngaacnccagtcacttt

SEQ ID NO: 91 - DNA, 16 bp, synthetic construct
  source 1..16: mol_type = other DNA
cggaagagtacacgtc

SEQ ID NO: 92 - DNA, 21 bp, synthetic construct
  misc_feature order(1..5,7,9..10,12,15..18,20..21): note = DNA
  misc_feature order(6,8,11,13..14,19): note = RNA
  source 1..21: mol_type = other DNA
agacgtgtgctcttccgatct

SEQ ID NO: 93 - DNA, 34 bp, synthetic construct; identical to SEQ ID NO: 47
  source 1..34: mol_type = other DNA

SEQ ID NO: 94 - DNA, 24 bp, synthetic construct
  source 1..24: mol_type = other DNA
gtcgtgtagggaaagagtgttttt

SEQ ID NO: 95 - DNA, 20 bp, synthetic construct
  source 1..20: mol_type = other DNA
acactctttccctacacgac

SEQ ID NO: 96 - DNA, 21 bp, synthetic construct
  source 1..21: mol_type = other DNA
gtgactggagttcagacgtgt

SEQ ID NO: 97 - DNA, 11 bp, synthetic construct
  source 1..11: mol_type = other DNA
actccagtcac

SEQ ID NO: 98 - DNA, 23 bp, synthetic construct
  misc_feature order(1..7,9,11..12,14,16..20,22..23): note = DNA
  misc_feature order(8,10,13,15,21): note = RNA
  source 1..23: mol_type = other DNA
tcagacgtgtgctcttccgatct

SEQ ID NO: 99 - RNA, 27 nt, synthetic construct
  source 1..27: mol_type = other RNA
gggctcggagatgtgtataagagacag

SEQ ID NO: 100 - DNA, 27 bp, synthetic construct
  source 1..27: mol_type = other DNA
ctgtctcttatacacatctccgagccc

SEQ ID NO: 101 - DNA, 18 bp, synthetic construct
  source 1..18: mol_type = other DNA
gacgctgccgacgatttt

SEQ ID NO: 102 - DNA, 24 bp, synthetic construct
  source 1..24: mol_type = other DNA
tcgtcggcagcgtcagatgtgtat

SEQ ID NO: 103 - DNA, 33 bp, synthetic construct
  source 1..33: mol_type = other DNA
tcgtcggcagcgtcagatgtgtataagagacag

SEQ ID NO: 104 - DNA, 34 bp, synthetic construct
  source 1..34: mol_type = other DNA
gtctcgtgggctcggagatgtgtataagagacag

SEQ ID NO: 105 - DNA, 22 bp, synthetic construct
  source 1..22: mol_type = other DNA
atacacatctccgagcccacga

SEQ ID NO: 106 - DNA, 14 bp, synthetic construct
  source 1..14: mol_type = other DNA
tcgtcggcagcgtc

SEQ ID NO: 107 - DNA, 15 bp, synthetic construct
  source 1..15: mol_type = other DNA
gtctcgtgggctcgg

SEQ ID NO: 108 - DNA, 28 bp, synthetic construct
  source 1..28: mol_type = other DNA
acgagacttttttatctcgtatgccgtc

SEQ ID NO: 109 - DNA, 55 bp, synthetic construct
  source 1..55: mol_type = other DNA
ctgtctcttatacacatctccgagcccacgagacttttttatctcgtatgccgtc

SEQ ID NO: 110 - DNA, 34 bp, synthetic construct; identical to SEQ ID NO: 104
  misc_feature order(1..3,5..6,8..11,13..18,20,22,24,26..34): note = Description of Artificial Sequence: Synthetic oligonucleotide
  misc_feature order(4,7,12,19,21,23,25): note = RNA
  source 1..34: mol_type = other DNA

SEQ ID NO: 111 - DNA, 34 bp, synthetic construct
  source 1..34: mol_type = other DNA
ctgtctcttatacacatctccgagcccacgagac

SEQ ID NO: 112 - DNA, 27 bp, synthetic construct
  source 1..27: mol_type = other DNA
ctgtctcttatacacatctccgagccc

SEQ ID NO: 113 - DNA, 30 bp, synthetic construct
  misc_difference 12..19: note = a, c, t, g, unknown or other
  source 1..30: mol_type = other DNA
gagatctacacnnnnnnnnacactctttcc

SEQ ID NO: 114 - DNA, 34 bp, synthetic construct
  misc_feature order(1..12,17..34): note = DNA
  misc_feature 13..16: note = RNA
  source 1..34: mol_type = other DNA
ttttttttttttttttaatgatacggcgaccacc

SEQ ID NO: 115 - DNA, 22 bp, synthetic construct
  source 1..22: mol_type = other DNA
ctacacgacgctcttccgatct

SEQ ID NO: 116 - DNA, 21 bp, synthetic construct
  source 1..21: mol_type = other DNA
gtgtagatctcggtggtcgcc

SEQ ID NO: 117 - DNA, 22 bp, synthetic construct
  source 1..22: mol_type = other DNA
gcgtcgtgtagggaaagagtgt

SEQ ID NO: 118 - DNA, 86 bp, synthetic construct
  misc_feature 13..16: note = RNA
  misc_feature order(1..12,17..45,54..86): note = DNA
  misc_difference 46..53: note = a, c, t, g, unknown or other
  source 1..86: mol_type = other DNA
ttttttttttttttttaatgatacggcgaccaccgagatctacacnnnnnnnnacactctttccctacacgacgctcttccgatct

SEQ ID NO: 119 - DNA, 29 bp, synthetic construct
  misc_difference 12..19: note = a, c, t, g, unknown or other
  source 1..29: mol_type = other DNA
gcatacgagatnnnnnnnngtgactggag

SEQ ID NO: 120 - DNA, 29 bp, synthetic construct
  misc_feature order(1..12,17..29): note = DNA
  misc_feature 13..16: note = RNA
  source 1..29: mol_type = other DNA
ttttttttttttttttcaagcagaagacg

SEQ ID NO: 121 - DNA, 24 bp, synthetic construct
  source 1..24: mol_type = other DNA
ttcagacgtgtgctcttccgatct

SEQ ID NO: 122 - DNA, 21 bp, synthetic construct
  source 1..21: mol_type = other DNA
atctcgtatgccgtcttctgc

SEQ ID NO: 123 - DNA, 82 bp, synthetic construct
  misc_feature order(1..12,17..40,49..82): note = DNA
  misc_feature 13..16: note = RNA
  misc_difference 41..48: note = a, c, t, g, unknown or other
  source 1..82: mol_type = other DNA
ttttttttttttttttcaagcagaagacggcatacgagatnnnnnnnngtgactggagttcagacgtgtgctcttccgatct

SEQ ID NO: 124 - DNA, 37 bp, synthetic construct
  misc_feature order(1..3,5..20,22..25,28..37): note = DNA
  misc_feature order(4,21,26): note = RNA
  source 1..37: mol_type = other DNA
agatcggaagagcacacgtctgaactccagtcacttt

SEQ ID NO: 125 - DNA, 37 bp, synthetic construct; identical to SEQ ID NO: 124
  misc_feature order(1..20,22..25,27..37): note = DNA
  misc_feature order(21,26): note = RNA
  source 1..37: mol_type = other DNA

SEQ ID NO: 126 - DNA, 13 bp, synthetic construct
  source 1..13: mol_type = other DNA
gctcttccgatct

SEQ ID NO: 127 - DNA, 13 bp, synthetic construct
  source 1..13: mol_type = other DNA
agatcggaagagc